(19) United States
(12) Patent Application Publication
(10) Pub. No.: US 2005/0288316 A1
(43) Pub. Date: Dec. 29, 2005

(54) Beta-carbolines as growth hormone secretagogue receptor (GHSR) antagonists

(76) Inventors: Roger Crossley, Caukuburg (GB); Annika Jenmalm Jensen, Uppsala (SE); Peter Brandt, Solna (SE); Ulf Bremberg, Uppsala (SE); Malin Graffner Nordberg, Uppsala (SE); Erik Ringberg, Uppsala (SE); Terry Ward, Coleville (GB)

Correspondence Address: FISH & RICHARDSON PC, P.O. BOX 1022, MINNEAPOLIS, MN, 55440-1022, US

(21) Appl. No.: 10/995,067

(22) Filed: Nov. 22, 2004

Related U.S. Application Data
(60) Provisional application No. 60/560,690, filed on Apr. 8, 2004.

(30) Foreign Application Priority Data
Nov. 20, 2003 (SE) 0303078-0

(51) Int. Cl. A61K 31/4747; A61K 31/407; C07D 491/14

(52) U.S. Cl. 514/278; 514/409; 548/413; 546/16

(57) ABSTRACT

The present invention relates to compounds of Formula (I): 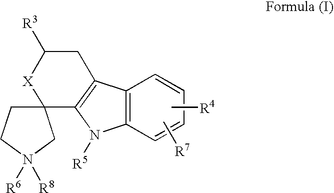
wherein R³-R⁸, X, and Y are as described herein, processes for preparing the compounds, pharmaceutical compositions comprising the compounds, and use of the compounds and compositions in the prophylaxis or treatment of a GHSR receptor-related disorder. Examples of such disorders are obesity and related disorders such as diabetes type II, dyslipidemia and the metabolic syndrome Prader-Willi syndrome, cardiovascular diseases such as atherosclerotic vascular disease, angina pectoris, myocardial infarction and stroke, acromegaly and cancer, in particular breast, lung, prostate, thyroid and endocrine pituary carcinomas.

### CROSS REFERENCE TO RELATED APPLICATIONS

This application claims the benefit of Swedish application number 0303078-0, filed on Nov. 20, 2003, and U.S. Provisional Application 60/560,690, filed on Apr. 8, 2004. The contents of both of these prior applications are incorporated herein by reference in their entireties.

### TECHNICAL FIELD

The present invention relates to novel compounds, to pharmaceutical compositions comprising the compounds, to processes for their preparation, the use of the compounds for the preparation of a medicament against GHSR receptor-related disorders, and methods for the prophylaxis and treatment of GHSR receptor-related disorders.

### BACKGROUND

Ghrelin is a 28 amino acid peptide firstly isolated from rat stomach extracts in 1999 (Kojima M, Hosoda H, Date Y, Nakazato M, Matsuo H and Kangawa K (1999) Ghrelin is a growth-hormone-releasing acylated peptide from stomach. Nature 402:656-660). The peptide resides an unusual n-octanoylation of the third residue Ser³. This post-translational modification is essential for the function of the peptide (Kojima et al., 1999) as well as for transport across the blood-brain barrier (Banks W A, Tschop M, Robinson S M and Heiman M L (2002) Extent and direction of ghrelin transport across the blood-brain barrier is determined by its unique primary structure. J Pharmacol Exp Ther 302:822-827). A G-protein coupled receptor that bound several small synthetic ligands with GH releasing effects known as growth hormone secretagogues (GHS) was cloned already in 1996. The new receptor was, therefore, named GHS-R (Howard A D, Feighner S D, Cully D F, Arena J P, Liberator P A, Rosenblum C I, Hamelin M, Hreniuk D L, Palyha O C, Anderson J, Paress P S, Diaz C, Chou M, Liu K K, McKee K K, Pong S S, Chaung L Y, Elbrecht A, Dashkevicz M, Heavens R, Rigby M, Sirinathsinghji D J, Dean D C, Melillo D G, Van der Ploeg L H and et al. (1996) A receptor in pituitary and hypothalamus that functions in growth hormone release. Science 273:974-977) and later identified as the receptor for ghrelin. Des-Gln¹⁴-ghrelin is another endogenous ligand for GHSR resulting from alternative splicing of the ghrelin gene (Hosoda H, Kojima M, Matsuo H and Kangawa K (2000) Purification and characterization of rat des-Gln14-Ghrelin, a second endogenous ligand for the growth hormone secretagogue receptor. J Biol Chem 275:21995-22000).

Ghrelin is a potent stimulator of adiposity and food intake in rodents (Tschop M, Smiley D L and Heiman M L (2000) Ghrelin induces adiposity in rodents. Nature 407:908-913, 2000; Wren A M, Small C J, Ward H L, Murphy K G, Dakin C L, Taheri S, Kennedy A R, Roberts G H, Morgan D G, Ghatei M A and Bloom S R (2000) The novel hypothalamic peptide ghrelin stimulates food intake and growth hormone secretion. Endocrinology 141:4325-4328; Nakazato M, Murakami N, Date Y, Kojima M, Matsuo H, Kangawa K and Matsukura S (2001) A role for ghrelin in the central regulation of feeding. Nature 409:194-198). Weight gain can be observed following both single daily subcutaneous doses and ICV dosing. The effect is present in GHRH deficient rodents pointing at a non-pituitary growth hormone mediated effect (Tschop et al., 2000). In contrast, lesions in the hypothalamic arcuate nucleus, a key area for regulation of energy homeostasis, abolished the orexigenic effects but not GH release of exogenously administered ghrelin in rats (Tamura H, Kamegai J, Shimizu T, Ishii S, Sugihara H and Oikawa S (2002) Ghrelin stimulates GH but not food intake in arcuate nucleus ablated rats. Endocrinology 143:3268-3275). Antagonists against GHSR decrease base-line food intake, weight gain, and energy expenditure suggesting that there is a tonic activation of this receptor that can be down regulated providing further support to the role of GHSR as a target for obesity related disease therapy. Further evidence for ghrelin as a key component in the regulation of the metabolism comes from the tissue distribution of the peptide and receptor. Messenger RNA and protein for ghrelin and GHS-R are abundant in many intestinal tissues but also in several brain tissues, in particular the arcuate nucleus of the hypothalamus (see Wang G, Lee H M, Englander E and Greeley G H, Jr. (2002) Ghrelin—not just another stomach hormone. Regul Pept 105:75-81 for a detailed review). In addition, GHS-R can also be detected in prostate cancers and several other tumours (Jeffery P L, Herington A C and Chopin L K (2003) The potential autocrine/paracrine roles of ghrelin and its receptor in hormone-dependent cancer Cytokine Growth Factor Rev 14:113-122).

Ghrelin is released in a pulsative manner characterized by a gradual increase before meal and rapid drops after suggesting a role in meal initiation and termination in man (Cummings D E, Purnell J Q, Frayo R S, Schmidova K, Wisse B E and Weigle D S (2001) A preprandial rise in plasma ghrelin levels suggests a role in meal initiation in humans. Diabetes 50:1714-1719). Several studies have confirmed the role of ghrelin in food intake and regulation of body composition in man (Wren A M, Seal L J, Cohen M A, Brynes A E, Frost G S, Murphy K G, Dhillo W S, Ghatei M A and Bloom S R (2001) Ghrelin enhances appetite and increases food intake in humans. J Clin Endocrinol Metab 86:5992). Healthy volunteers ate significantly more after Systemic administration of ghrelin. The effect was still significant 24 hours after the injection. This along with the fact that chronic administration in rodents result in weight gain, indicate a possibility for long-term treatment of human obesity and other metabolic disease.

Apart from the regulatory effects on food intake and metabolism, Ghrelin may also possess anxiogenic and cardiovascular effects. Ghrelin, when injected ICV or peripherally, dose-dependently decreases time spent in the open arm of an elevated plus-maze as well as number of entries (Asakawa A, Inui A, Kaga T, Yuzuriha H, Nagata T, Fujimiya M, Katsuura G, Makino S, Fujino M A and Kasuga M (2001) A role of ghrelin in neuroendocrine and behavioral responses to stress in mice. Neuroendocrinology 74:143-147). The anxiogenic effect could be blocked by a corticotropin-releasing hormone (CRH) receptor antagonist pointing at an involvement of ghrelin in the hypothalamic-pituitary-adrenal System. Furthermore, chronic administration of ghrelin is associated with improved prognosis after heart failure (Nagaya N, Uematsu M, Kojima M, Ikeda Y, Yoshihara F, Shimizu W, Hosoda H, Hirota Y, Ishida H, Mori H and Kangawa K (2001c) Chronic administration of ghrelin improves left ventricular dysfunction and attenuates development of cardiac cachexia in rats with heart failure. Circulation 104:1430-1435).

Prader-Willi syndrome (PWS) is the most common form of human syndromic obesity. It is characterized by severe obesity, hyperphagia, hypogonadism, GH deficiency, neonatal hypotonia, dysmophic features and cognitive impairment. Although the genetic basis of PWS involves imprinting disorders of several genes on chromosome 15, mediators of the pheontype are unknown. As ghrelin affects both appetite and GH secretion, and both are abnormal in PWS-PWS patients have high fasting-ghrelin concentrations.

Interventions that inhibit the actions of circulating ghrelin, such as ghrelin receptor antagonists could be beneficial in the treatment of obesity due to PWS.

Ghrelin is present in pancreatic alpha cells of the rat, where it may act in a paracrine/autocrine fashion to regulate insulin secretion. When administered acutely into human normal young volunteers, ghrelin induces hyperglycemia as well as reduces serum levels of insulin. Thus, ghrelin receptor modulators may be beneficial in the treatment of type II diabetes.

In summary, ghrelin and other GHSR targeting compounds are effective in both acute and long term-regulation of food intake and energy expenditure. Thus, an antagonist would be highly interesting, making the GHSR one of the most promising targets for treatment of metabolic diseases.

### SUMMARY

In one aspect, this invention relates to compounds of Formula (I) 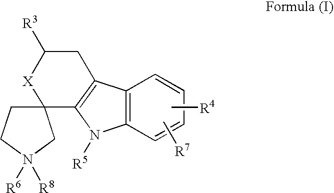
wherein
X is O or NR, wherein R is selected from hydrogen, C₁₋₆-alkyl, hydroxy-C₁₋₆-alkyl, C₂₋₆-alkenyl, C₂₋₆-acyl, hydroxy-C₂₋₆-acyl, C₁₋₆-alkylcarbamoyl, di-C₁₋₆-alkylcarbamoyl, C₂₋₆-alkenylcarbamoyl, C₃₋₈-cycloalkylcarbamoyl, C₁₋₆-alkylsulfonyl, N-glycylcarbonyl, C₁₋₆-alkyl ester of N-glycylcarbonyl, C₁₋₆-alkyl ester of N-glycylacetyl, carbamoyl-C₁₋₆-alkyl, N—C₁₋₆-alkylcarbamoyl-C₁₋₆-alkyl, N,N—C₁₋₆-dialkylcarbamoyl-C₁₋₆-alkyl, N,N—C₁₋₆-dialkylcarbamoylamino-C₁₋₆-alkyl, C₁₋₆-alkoxy-C₂₋₆-acylamino-C₁₋₆-alkyl, 3-amino-1,2-dioxocyclobut-3-ene-4-ylamino-C₁₋₆-alkyl, 3-C₁₋₆-alkoxy-1,2-dioxocyclobut-3-ene-4-ylamino-C₁₋₆-alkyl, cyano-C₁₋₆-alkyl, C₁₋₆-alkoxyhydroxyalkyl, carboxy-C₁₋₆-alkyl, C₁₋₆-alkoxycarbonyl-C₁₋₆-alkyl, C₁₋₆-alkoxy-C₁₋₆-alkyl, C₁₋₆-alkoxy-C₁₋₆-alkoxy-C₁₋₆-alkyl, aryl-C₁₋₆-alkylamino-C₂₋₆-acyl, C₁₋₆-alkoxycarbonyl-C₁₋₆-alkylamino-C₂₋₆-acyl, carboxy-C₁₋₆-alkylamino-C₂₋₆-acyl, C₂₋₆-acyl-C₂₋₆-acyl, aryloxy-C₁₋₆-alkyl, C₁₋₆-alkylsulfonylamino-C₁₋₆-alkyl, C₁₋₆-alkoxycarbonyl-C₂₋₆-acyl, C₁₋₆-alkoxy-C₂₋₆-acyl, C₁₋₆-alkylthio-C₂₋₆-acyl, di-C₁₋₆-alkylamino-C₂₋₆-acyl, heteroarylcarbamoyl, C₁₋₆-alkoxycarbonyl, heteroaryl-C₂₋₆-acyl, C₁₋₆-alkylsulfonyl-C₂₋₆-acyl, heterocyclyl-C₂₋₆-acyl, C₁₋₆-alkoxy-C₁₋₆-alkylamino-C₂₋₆-acyl, carboxy-C₂₋₆-acyl, amino-C₂₋₆-acyl, C₁₋₆-alkylamino-C₂₋₆-acyl, carbamoyl-C₁₋₆-alkylamino-C₂₋₆-acyl, heterocyclyl-C₁₋₆-alkyl, heteroaryl-C₁₋₆-alkyl, carbamoylamino-C₁₋₆-alkyl, hydroxy-C₂₋₆-acylcarbamoyl, C₁₋₆-alkylcarbamoyl-C₁₋₆-alkylamino-C₁₋₆-alkyl, C₁₋₆-alkoxycarbonyl-C₁₋₆-alkylamino-C₁₋₆-alkyl, amino-C₂₋₆-acylamino-C₂₋₆-acyl, C₁₋₆-alkoxy-C₂₋₆-acylamino-C₂₋₆-acyl, amino-C₂₋₆-acylamino-C₁₋₆-alkyl, amino-C₂₋₆-acylamino-C₁₋₆-alkyl, heterocyclylcarbonylamino-C₁₋₆-alkyl, C₂₋₆-acylamino-C₁₋₆-alkyl, amino-C₂₋₆-acylamino-C₂₋₆-acyl, C₂₋₆-acylamino-C₂₋₆-acyl, hydroxy-C₁₋₆-alkylamino-C₂₋₆-acyl, C₁₋₆-alkoxycarbonyl-C₁₋₆-alkyl, amino-C₁₋₆-alkyl, carboxy-C₁₋₆-alkyl, 2-(3-hydroxy-1,2-dioxocyclobut-3-ene-4-yl)amino-C₁₋₆-alkyl, heteroarylcarbonylamino-C₁₋₆-alkyl, carboxyamino-C₁₋₆-alkyl, N,N-di-C₁₋₆-alkylamino-C₂₋₆-acylamino-C₁₋₆-alkyl, dihydroxy-C₁₋₆-alkyl, C₂₋₆-acylcarbonyl, C₁₋₆-alkoxybenzyl, and CO—CH₂—R⁶, wherein the aryl group is optionally substituted by one or more of C₁₋₆-alkoxy, the heteroaryl group is optionally substituted by one or more of C₁₋₆-alkyl and the heterocyclyl is optionally substituted by one or more of oxo;
Y is O, S, NH, CH₂, CO, or a single bond;
R¹ is hydrogen or C₁₋₃-alkyl;
R² is C₃₋₈-cycloalkyl, hexahydro-N-phthalimidyl, an aryl or heteroaryl ring optionally substituted by one or more of C₁₋₆-alkyl, halogen, methylenedioxy, C₁₋₆-alkoxy, halo-C₁₋₆-alkoxy, C₁₋₆-alkylsulfonyl, or cyano;
R³ is hydrogen;
R⁴ is hydrogen, C₁₋₆-alkyl, C₁₋₆-alkoxy, or halogen;
R⁵ is hydrogen or C₁₋₆-alkyl;
R⁶ is either bonded to X via a methylene and a carbonyl group, or R⁶ is hydroxy-C₁₋₆-alkyl or C₁₋₆-alkoxycarbonyl-C₁₋₆-alkyl;
R⁷ is hydrogen or C₁₋₆-alkyl;
R⁸ is —CH(R¹)—(CHOH)ₘ—[(CH(R⁹)]ₙ—Y—R², —CH(R¹)—(CH═CH)ₒ—Y—R², hydrogen or C₁₋₆-alkyl;
R⁹ is hydrogen or C₁₋₆-alkyl;
m is 0 or 1;
n is 0, 1, or 2;
o is 0 or 1;
with the proviso that when Y is a single bond, then R² is 5-methyl-3-indolyl, 3-indolyl, cyclohexyl, 2,3-dihydro-1,4-benzodioxin-2-yl, phenyl, 2-methoxyphenyl, 4-difluoromethoxyphenyl, or 3,4-methylenedioxyphenyl;
and pharmaceutically acceptable salts, hydrates, solvates, geometrical isomers, tautomers, optical isomers, and prodrug forms thereof.

It is preferred that R is selected from acetyl, allyl, allylcarbamoyl, aminoacetyl, 2-(3-amino-1,2-dioxocyclobut-3-ene-4-ylamino)ethyl, 3-amino-3-methyl-n-butyryl, benzylaminoacetyl, n-butylcarbamoyl, carbamoylmethyl, carbamoylmethylaminoacetyl, 3-carbamoyl-n-propyl, carbethoxy, carbethoxyacetyl, 4-carbethoxy-n-butyl, carbethoxymethyl, 3-carbethoxy-n-propyl, carbomethoxyacetyl, 4-carbomethoxy-n-butyryl, 4-carboxy-n-butyl, 3-carboxy-n-propionyl, 3-carboxy-n-propyl, 3-cyano-n-propyl, cyclohexylcarbamoyl, N,N-diethylcarbamoylmethyl, diisopropylaminoacetyl, 3,4-dimethoxybenzylaminoacetyl, dimethylaminoacetyl, 2-(N,N-dimethylcarbamoylamino)ethyl, 3,5-dimethylisoxazol-4-ylcarbamoyl, 1,4-dioxo-n-pentyl, 2-(3-ethoxy-1,2-dioxocyclobut-3-ene-4-ylamino)ethyl, ethylcarbamoyl, 4ethylcarbamoyl-n-butyl, 3-ethylcarbamoyl-n-propyl, ethyl ester of N-glycylacetyl, ethyl ester of N-glycylcarbonyl, N-ethyl-N-methylcarbamoyl, ethylthioacetyl, N-glycylacetyl, N-glycylcarbonyl, hydrogen, hydroxyacetyl, 2-hydroxyisobutyl, 2-hydroxyethyl, 2-hydroxy-3-methoxy-n-propyl, 2-hydroxy-n-propyl, 1-imidazolylacetyl, methoxyacetyl, 2-(methoxyacetylamino)ethyl, 2-(2-methoxyethoxy)ethyl, 2-methoxyethylaminoacetyl, 3-methoxy-n-propyl, methyl, methylaminoacetyl, methylsulfonyl, methylsulfonylacetyl, 2-methylsulfonylaminoethyl, 4-morpholinylacetyl, 2-(4-morpholinyl)ethyl, 3-oxo-1-piperazinylacetyl, 2-phenoxyethyl, 1-piperazinylacetyl, 2-pyridylmethyl, 2-thienylcarbamoyl, 2-carbamoylaminoethyl, hydroxyacetylcarbamoyl, 2-(N-methylcarbamoylmethylamino)ethyl, 2-carbomethoxymetylaminoethyl, 2-amino-2-methylpropionamidoacetyl, methoxyacetylaminoacetyl, 2-(2-amino-2-methylpropionamido)ethyl, 2-aminoacetylaminoethyl, 2-(4-morpholinylcarbonylamino)ethyl, 2-acetylaminoethyl, aminoacetylaminoacetyl, acetylaminoacetyl, 2-hydroxyethylaminoacetyl, carbomethoxymethyl, 2-aminoethyl, carboxymethyl, 2-(3-hydroxy-1,2-dioxocyclobut-3-ene-4-yl)aminoethyl, 2-(2-furylcarbonylamino)ethyl, 2-(5-isoxazolylcarbonylamino)ethyl, 2-carboxyaminoethyl, 2-(2-morpholinylcarbonylamino)ethyl, 2-N,N-dimethylaminoacetylaminoethyl, 4-phenoxy-n-butyl, 2,3-dihydroxy-n-propyl, acetylcarbonyl, and 4-methoxybenzyl.

It is preferred that R¹ is hydrogen or methyl.

It is preferred that R² is selected from N-hexahydrophthalimidyl, cyclohexyl, 2,3-dihydro-1,4-benzodioxin-2-yl; a phenyl or indole ring optionally substituted by one or more of methyl, ethyl, fluoro, chloro, methylenedioxy, difluoromethoxy, methylsulfonyl, methoxy, cyano, isopropyl; and naphthyl.

It is more preferred that R² is selected from N-hexahydrophthalimidyl, cyclohexyl, 2,3-dihydro-1,4-benzodioxin-2-yl, phenyl, 2-methylphenyl, 2-fluorophenyl, 4-fluorophenyl, 3-chlorophenyl, 4-chlorophenyl, 3,4-methylenedioxyphenyl, 2-methoxyphenyl, 4-methoxyphenyl, 2-cyanophenyl, 4-cyanophenyl, 4-ethylphenyl, 4difluoromethoxyphenyl, 4-methylsulfonylphenyl, 4-carbamoylphenyl, indolyl, 5-methyl-3-indolyl, 3-methoxyphenyl, 3-isopropylphenyl, and naphthyl.

It is preferred that R⁴ is selected from hydrogen, bromo, fluoro, methyl, and methoxy.

It is preferred that R⁵ is hydrogen or methyl.

It is preferred that R⁶ is hydroxymethyl or carbomethoxymethyl.

It is preferred that R⁷ is hydrogen or methyl.

It is preferred that R⁸ is hydrogen or methyl.

It is preferred that R⁹ is hydrogen or methyl.

Especially preferred compounds are given in Examples 4-21, 25-35, 37-58, 60-65, 67-70, 72-84, 86, 87, 89-100, 102-122, 124-129, 131, 132, 134-137, 139-148, 150-159, 162, 163, 165, 166, 168, 169, 171, 172, 174-181, 184, 199-201, 206, 213-215, 219, 220, 223, 226, 228, 230, 232, 233, and 234.

In another aspect, this invention relates to processes for the preparation of a compound as described above, which process comprises at least one of the following reaction sequences a₁, a₂, a₃, b-p): 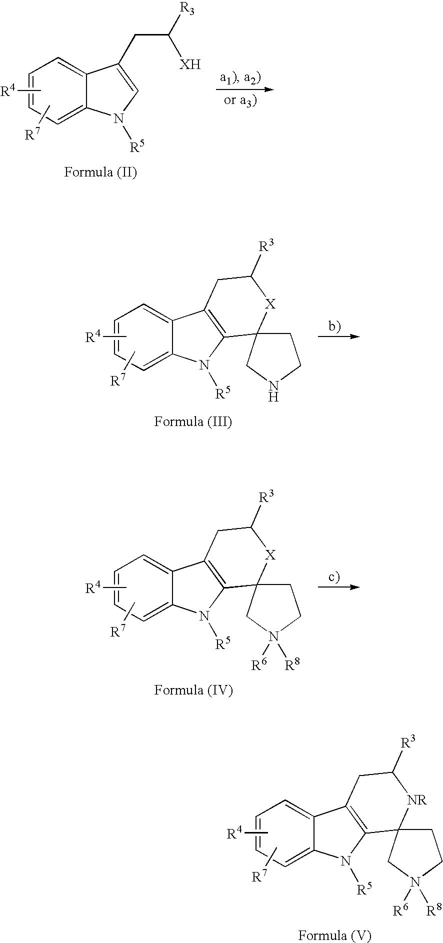
a1) the reaction of a compound of Formula (II) with N-Boc-3-pyrrolidinone and subsequent acidic hydrolysis;
a2) the reaction of a compound of Formula (II) with N-benzyl-3-pyrrolidinone and the subsequent hydrogenolysis;
a3) the reaction of a compound of Formula (II) with 1-(phenoxyethyl)pyrrolidin-3-one;
b) the reaction of a compound of Formula (III) with R²—Y—[(CH(R⁹)]ₙ—(CHOH)ₘ—CH(R¹)-LG or R₂—Y—(CH═CH)ₒ—CH(R¹)-LG;
c) reaction of a compound of Formula (IV) with acetic anhydride, an isocyanate or an alkylating agent;
wherein R, Y, R¹, R², R³, R⁴, R⁵, R⁶, R⁷, R⁸, R⁹, m, n, and o are as defined above and LG is a leaving group; 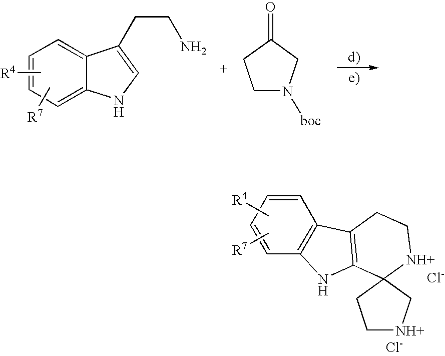
d) treatment with acetic acid;
e) treatment with hydrochloric acid in dioxane; 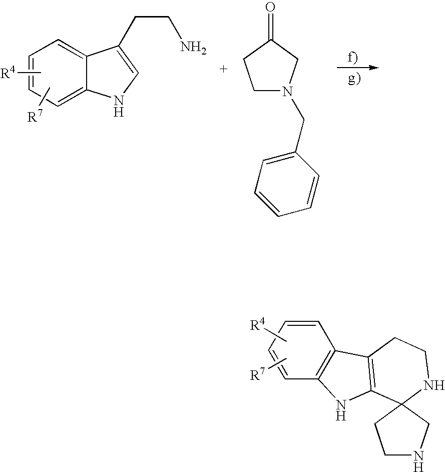
f) treatment with acetic acid;
g) treatment with hydrogen in the presence of palladium hydroxide; 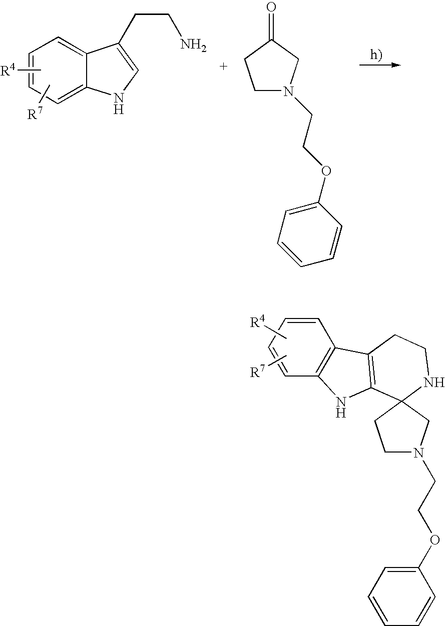
h) treatment with acetic acid; 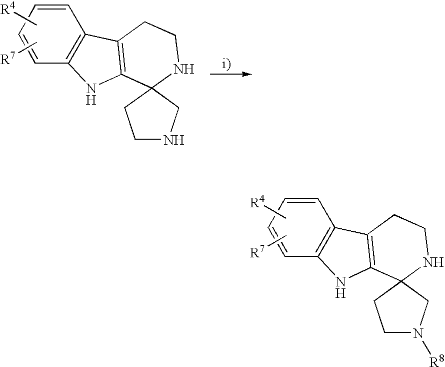
i) treatment with R⁸—Br, R⁹—Cl or R⁸—OMs; 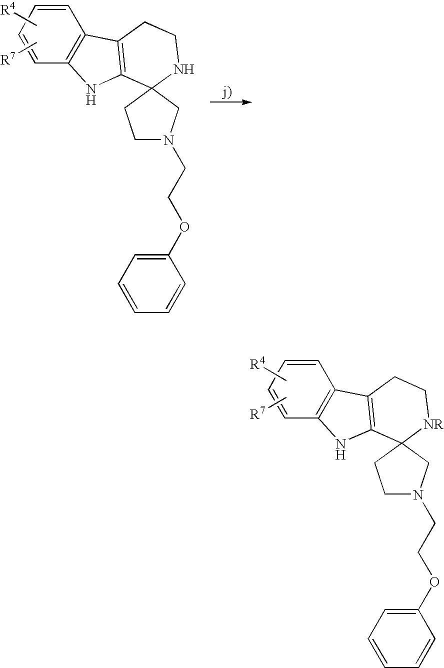
j) treatment with an isocyanate; 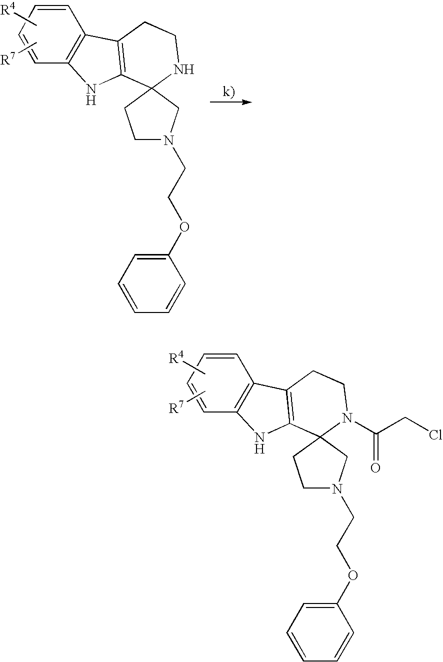
k) acylation with chloroacetyl chloride; 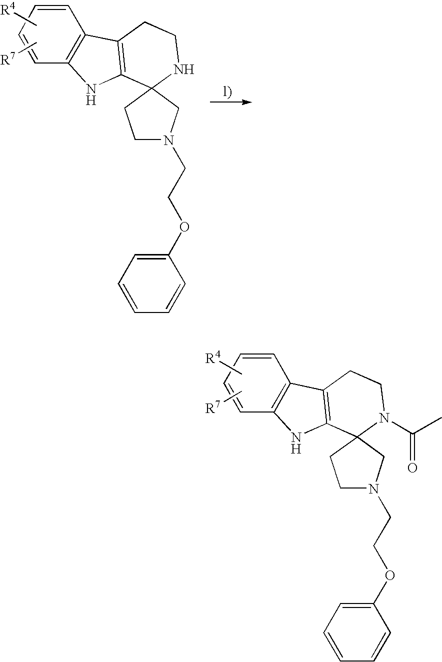
l) acylation with acetic anhydride; 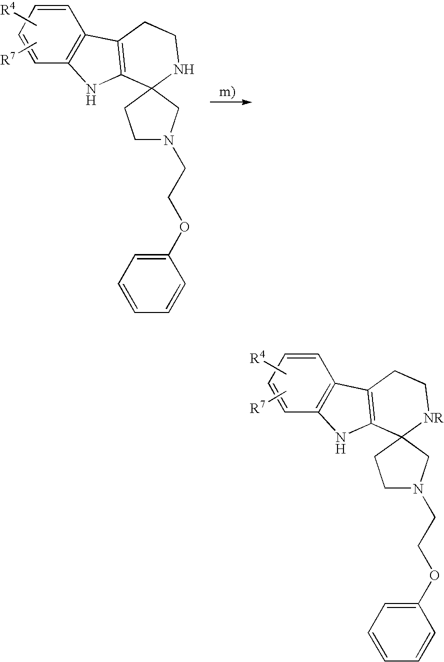
m) alkylation with R-LG; 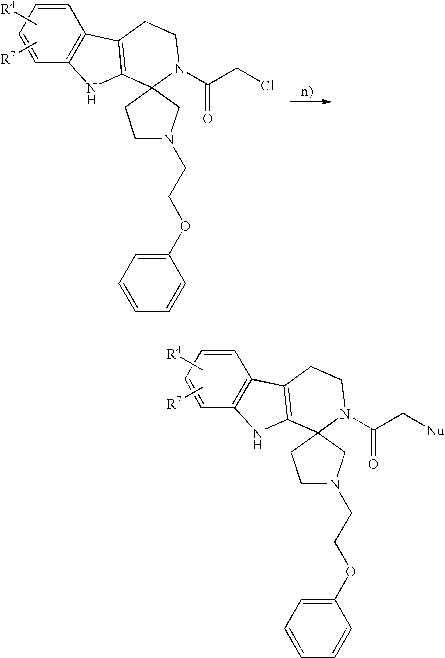
n) reaction with a nucleophile Nu; 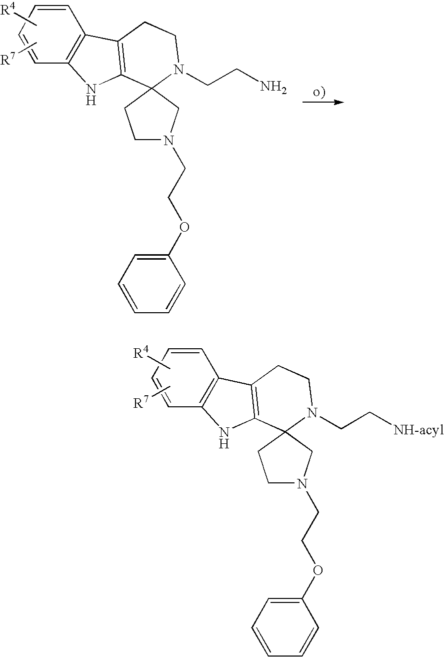
o) reaction with a carboxylic acid in the presence of a coupling agent; 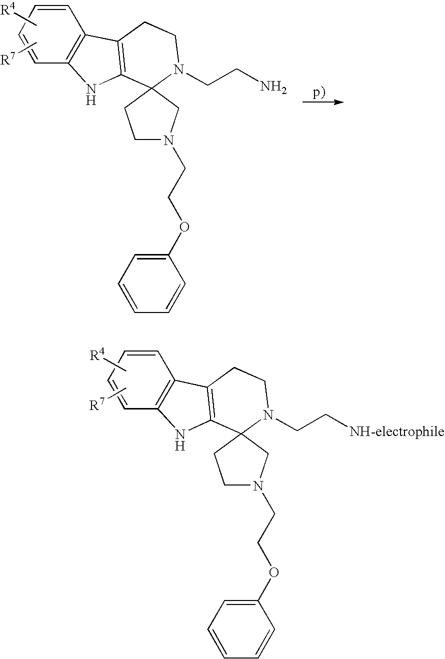
p) reaction with an electrophile.

In a further aspect, this invention relates to compounds as mentioned above for use in therapy, especially for use in the prophylaxis or treatment of a GHSR receptor-related disorder.

In one aspect, this invention relates to pharmaceutical formulations comprising a compound as mentioned above as active ingredient, in combination with a pharmaceutically acceptable diluent or carrier, especially for use in the prophylaxis or treatment of a GHSR receptor-related disorder.

In another aspect, this invention relates to methods for treating a human or animal subject suffering from a GHSR receptor-related disorder. The method can include administering to a subject (e.g., a human or an animal, dog, cat, horse, cow) in need thereof an effective amount of one or more compounds of any of the formulae herein, their salts, or compositions containing the compounds or salts.

The methods delineated herein can also include the step of identifying that the subject is in need of treatment of the GHSR receptor-related disorder. Identifying a subject in need of such treatment can be in the judgment of a subject or a health care professional and can be subjective (e.g., opinion) or objective (e.g., measurable by a test or diagnostic method).

In a further aspect, this invention relates to methods for the prophylaxis of a GHSR receptor-related disorder, which comprises administering to a subject in need of such treatment an effective amount of a compound as mentioned above.

In one aspect, this invention relates to methods for modulating (e g, promoting or inhibiting) GHSR receptor activity, which comprises administering to a subject in need of such treatment an effective amount of a compound as mentioned above.

In another aspect, this invention relates to methods for suppressing food intake, which comprises administering to a subject in need of such treatment an effective amount of a compound as mentioned above.

In a further aspect, this invention relates to methods for suppressing appetite, which comprises administering to a subject in need of such treatment an effective amount of a compound as mentioned above.

In one aspect, this invention relates to methods for reducing weight, which comprises administering to a subject in need of such treatment an effective amount of a compound as mentioned above.

In another aspect, this invention relates to methods for reducing weight gain, which comprises administering to a subject in need of such treatment an effective amount of a compound as mentioned above.

In a further aspect, this invention relates to methods for increasing food intake, which comprises administering to a subject in need of such treatment an effective amount of a compound as mentioned above.

In one aspect, this invention relates to methods for increasing appetite, which comprises administering to a subject in need of such treatment an effective amount of a compound as mentioned above.

In another aspect, this invention relates to methods for increasing weight, which comprises administering to a subject in need of such treatment an effective amount of a compound as mentioned above.

In a further aspect, this invention relates to methods for increasing weight gain, which comprises administering to a subject in need of such treatment an effective amount of a compound as mentioned above.

In one aspect, this invention relates to the use of a compound as mentioned above for the manufacture of a medicament for use in the prophylaxis or treatment of a GHSR receptor-related disorder.

The compounds as mentioned above may be agonists, partial agonists, partial antagonists, or antagonists for the GHSR receptor.

Examples of GHSR receptor-related disorders are obesity and related disorders such as diabetes type II, dyslipidemia and the metabolic syndrome Prader-Willi syndrome, cardiovascular diseases such as atherosclerotic vascular disease, angina pectoris, myocardial infarction and stroke, acromegaly and cancer, in particular breast, lung, prostate, thyroid and endocrine pituary carcinomas.

The compounds and compositions are useful for treating diseases, including obesity and related disorders such as diabetes type II, dyslipidemia and the metabolic syndrome Prader-Willi syndrome, cardiovascular diseases such as atherosclerotic vascular disease, angina pectoris, myocardial infarction and stroke, acromegaly and cancer, in particular breast, lung, prostate, thyroid and endocrine pituary carcinomas. In one aspect, the invention relates to a method for treating or preventing an aforementioned disease comprising administering to a subject in need of such treatment an effective amount of a compound or composition delineated herein.

The details of one or more embodiments of the invention are set forth in the accompanying drawings and the description below. Other features and advantages of the invention will be apparent from the description and from the claims.

### DETAILED DESCRIPTION

Definitions

The following definitions shall apply throughout the specification and the appended claims.

Unless otherwise stated or indicated, the term “C₁₋₆-alkyl” denotes a straight or branched alkyl group having from 1 to 6 carbon atoms. Examples of said lower alkyl include methyl, ethyl, n-propyl, iso-propyl, n-butyl, iso-butyl, sec-butyl, t-butyl and straight- and branched-chain pentyl and hexyl. For parts of the range “C₁₋₆-alkyl” all subgroups thereof are contemplated such as C₁₋₅-alkyl, C₁₋₄-alkyl, C₁₋₃-alkyl, C₁₋₂-alkyl, C₂₋₆-alkyl, C₂₋₅-alkyl, C₂₋₄-alkyl, C₂₋₃-alkyl, C₃₋₆-alkyl, C₄₋₅-alkyl, etc. “C₁₋₆-alkylcarbamoyl” means a carbamoyl group substituted by a C₁₋₆-alkyl group. “C₁₋₆-alkyl ester of N-glycylcarbonyl” means a that a carbonyl group is bonded the N-terminal of a C₁₋₆-alkyl ester of glycine. “C₁₋₆-alkylsulfonyl” means a sulfonyl group bonded to a C₁₋₆-alkyl group.

Unless otherwise stated or indicated, the term “C₂₋₆-alkenyl” denotes a straight or branched alkenyl group having from 2 to 6 carbon atoms. Examples of said alkenyl include vinyl, allyl, 1-butenyl, 1-pentenyl, and 1-hexenyl. For parts of the range “C₂₋₆-alkenyl” all subgroups thereof are contemplated such as C₂₋₅-alkenyl, C₂₋₄-alkenyl, C₂₋₃-alkenyl, C₃₋₆-alkenyl, C₃₋₅-alkenyl, C₃₋₄-alkenyl, C₄₋₆-alkenyl, C₄₋₅-alkenyl, etc. “C₂₋₆-alkenylcarbamoyl” means a carbamoyl group substituted by a C₂₋₆-alkenyl group.

Unless otherwise stated or indicated, the term “C₁₋₆-acyl” denotes a straight or branched acyl group having from 1 to 6 carbon atoms. Examples of said lower acyl include formyl, acetyl, propionyl, n-butyryl, 2-methylpropionyl, n-pentoyl, and n-hexoyl. For parts of the range “C₁₋₆-acyl” all subgroups thereof are contemplated such as C₁₋₅-acyl, C₁₋₄-acyl, C₁₋₃-acyl, C₁₋₂-acyl, C₂₋₆-acyl, C₂₋₅-acyl, C₂₋₄-acyl, C₂₋₃-acyl, C₃₋₆-acyl, C₄₋₅-acyl, etc.

Unless otherwise stated or indicated, the term “C₃₋₈-cycloalkyl” denotes a cyclic alkyl group having a ring size from 3 to 8 carbon atoms. Examples of said cycloalkyl include cyclopropyl, cyclobutyl, cyclopentyl, cyclohexyl, methylcyclohexyl, cycloheptyl, and cyclooctyl. For parts of the range “C₃₋₈-cycloalkyl” all subgroups thereof are contemplated such as C₃₋₇-cycloalkyl, C₃₋₆-cycloalkyl, C₃₋₅-cycloalkyl, C₃₋₄-cycloalkyl, C₄₋₈-cycloalkyl, C₄₋₇-cycloalkyl, C₄₋₆-cycloalkyl, C₄₋₅-cycloalkyl, C₅₋₇-cycloalkyl, C₆₋₇-cycloalkyl, etc. “C₃₋₈-cycloalkylcarbamoyl” means a carbamoyl group substituted by a C₃₋₈-cycloalkyl group.

Unless otherwise stated or indicated, the term “C₁₋₆ alkoxy” denotes a straight or branched alkoxy group having from 1 to 6 carbon atoms. Examples of said lower alkoxy include methoxy, ethoxy, n-propoxy, iso-propoxy, n-butoxy, iso-butoxy, sec-butoxy, t-butoxy and straight- and branched-chain pentoxy and hexoxy. For parts of the range “C₁₋₆-alkoxy” all subgroups thereof are contemplated such as C₁₋₅-alkoxy, C₁₋₄-alkoxy, C₁₋₃-alkoxy, C₁₋₂-alkoxy, C₂₋₆-alkoxy, C₂₋₅-alkoxy, C₂₋₄-alkoxy, C₂₋₃-alkoxy, C₃₋₆-alkoxy, C₄₋₅-alkoxy, etc.

Unless otherwise stated or indicated, the term “halogen” shall mean fluorine, chlorine, bromine or iodine.

Unless otherwise stated or indicated, the term “aryl” refers to a hydrocarbon ring System having at least one aromatic ring. Examples of aryls are phenyl, pentalenyl, indenyl, indanyl, isoindolinyl, chromanyl, naphthyl, fluorenyl, anthryl, phenanthryl and pyrenyl. The aryl rings may optionally be substituted with C₁₋₆-alkyl. Examples of substituted aryl groups are 2-methylphenyl and 3-methylphenyl. Likewise, aryloxy refers to an aryl group bonded to an oxygen atom.

The term “heteroaryl” means in the present description a monocyclic, bi- or tricyclic aromatic ring System (only one ring need to be aromatic) having from 5 to 14, preferably 5 to 10 ring atoms such as 5, 6, 7, 8, 9 or 10 ring atoms (mono- or bicyclic), in which one or more of the ring atoms are other than carbon, such as nitrogen, sulfur, oxygen and selenium as part of the ring System. Examples of such heteroaryl rings are pyrrole, imidazole, thiophene, furan, thiazole, isothiazole, thiadiazole, oxazole, isoxazole, oxadiazole, pyridine, pyrazine, pyrimidine, pyridazine, pyrazole, triazole, tetrazole, chroman, isochroman, quinoline, quinoxaline, isoquinoline, phthalazine, cinnoline, quinazoline, indole, isoindole, indoline (i e 2,3-dihydroindole), isoindoline (i e 1,3-dihydroisoindole), benzothiophene, benzofuran, 2,3-dihydrobenzofuran, isobenzofuran, benzodioxole, benzothiadiazole, benzotriazole, benzoxazole, 2,1,3-benzoxadiazole, benzopyrazole, 2,1,3-benzothiazole, 2,1,3-benzoselenadiazole, benzimidazole, indazole, benzodioxane, 2,3-dihydro-1,4-benzodioxine, indane, 1,2,3,4-tetrahydroquinoline, 3,4-dihydro-2H-1,4-benzoxazine, 1,5-naphthyridine, 1,8-naphthyridine, pyrido[3,2-b]thiophene, acridine, fenazine and xanthene.

The term “heterocyclic” and “heterocyclyl” in the present description is intended to include unsaturated as well as partially and fully saturated mono-, bi- and tricyclic rings having from 4 to 14, preferably 4 to 10 ring atoms having one or more heteroatoms (e.g., oxygen, sulfur, or nitrogen) as part of the ring System and the reminder being carbon, such as, for example, the heteroaryl groups mentioned above as well as the corresponding partially saturated or fully saturated heterocyclic rings. Exemplary saturated heterocyclic rings are azetidine, pyrrolidine, piperidine, piperazine, morpholine, thiomorpholine, 1,4-oxazepane, azepane, phthalimide, indoline, isoindoline, 1,2,3,4-tetrahydroquinoline, 1,2,3,4-tetrahydroisoquinoline, hexahydroazepine, 3,4-dihydro-2(1H)isoquinoline, 2,3-dihydro-1H-indole, 1,3-dihydro-2H-isoindole, azocane, 1-oxa-4-azaspiro[4.5]dec-4-ene, decahydroisoquinoline, 1,2-dihydroquinoline, and 1,4-diazepane.

The term “leaving group” refers to a group to be displaced from a molecule during a nucleophilic displacement reaction. Examples of leaving groups are iodide, bromide, chloride, methanesulfonate (mesylate; OMs), hydroxy, methoxy, thiomethoxy, tosyl, or suitable protonated forms thereof (e.g., H₂O, MeOH), especially bromide and methanesulfonate.

The term “alkylating agent” refers to a compound containing one or more alkyl groups which can be added to another compound. Examples of alkylating agents include, but are not limited to, iodomethane, iodoethane, 1-iodopropane, 2-iodopropane, straight- and branched-iodobutane, iodopentane, iodohexane, bromomethane, bromoethane, 1-bromopropane, 2-bromopropane, straight- and branched-bromobutane, bromopentane, bromohexane, allyl bromide, ethyl methanesulfonate, methyl methanesulfonate, and propyl methanesulfonate.

“Pharmaceutically acceptable” means being useful in preparing a pharmaceutical composition that is generally safe, non-toxic and neither biologically nor otherwise undesirable and includes being useful for veterinary use as well as human pharmaceutical use.

“Treatment” as used herein includes prophylaxis of the named disorder or condition, or amelioration or elimination of the disorder once it has been established.

“An effective amount” refers to an amount of a compound that confers a therapeutic effect on the treated subject. The therapeutic effect may be objective (i.e., measurable by some test or marker) or subjective (i.e., subject gives an indication of or feels an effect).

The term “prodrug forms” means a pharmacologically acceptable derivative, such as an ester or an amide, which derivative is biotransformed in the body to form the active drug. Reference is made to Goodman and Gilman's, The Pharmacological basis of Therapeutics, 8^{th} ed., Mc-Graw-Hill, Int. Ed. 1992, “Biotransformation of Drugs”, p. 13-15.

The term “nucleophile” means a compound having nucleophilic properties which can displace leaving groups in another compound. Examples of nucleophiles are water, alcohols, amines, phenolates, azides, etc.

The term “electrophile” means a compound having electrophilic properties which compound is prone to attack by a nucleophile. Examples of electrophiles are acyl halides, acyl anhydrides, carboxylic esters, alkyl halides, etc.

The term “coupling agent” means a compound in the presence of which a coupling reaction may proceed such as the reaction between an amine and a carboxylic acid to give an amide. Examples of coupling agents are PyBOP, CDI, etc.

When two of the above-mentioned terms are used together, it is intended that the latter group is substituted by the former. For example, C₃₋₆-alkenylcarbamoyl means a carbamoyl group that is substituted by a C₃₋₆-alkenyl group. Likewise, C₁₋₆-alkylsulfonyl means a sulfonyl group that is substituted by a C₁₋₆-alkyl group.

The following abbreviations have been used:
ACN means acetonitrile,
AcOH means acetic acid,
CDI means carbonyl diimidazole,
CHO means Chinese hamster ovary,
DCM means dichloromethane,
DEA means di ethyl amine,
DEPT means distortion enhancement polarisation transfer,
DIPEA means N,N-diisopropylethylamine
DMAP means N,N-dimethylaminopyridine
DMF means dimethylformamide,
DMSO means dimethyl sulfoxide,
ELS means electron light scattering,
HPLC means high performance liquid chromatography,
IPA means isopropylamine,
o/n means overnight,
PyBOP means (benzotriazol-1-yloxy)tripyrrolidinophosphonium
hexafluorophosphate,
rt means room temperature,
Rt means retention time,
TEA means triethylamine,
TFA means trifluoroacetic acid,
THF means tetrahydrofuran,
TLC means thin layer chromatography.

All isomeric forms possible (pure enantiomers, diastereomers, tautomers, racemic mixtures and unequal mixtures of two enantiomers) for the compounds delineated are within the scope of the invention. Such compounds can also occur as cis- or trans-, E- or Z-double bond isomer forms. All isomeric forms are contemplated.

The compounds of Formula (I) may be used as such or, where appropriate, as pharmacologically acceptable salts (acid or base addition salts) thereof. The pharmacologically acceptable addition salts mentioned above are meant to comprise the therapeutically active non-toxic acid and base addition salt forms that the compounds are able to form. Compounds that have basic properties can be converted to their pharmaceutically acceptable acid addition salts by treating the base form with an appropriate acid. Exemplary acids include inorganic acids, such as hydrogen chloride, hydrogen bromide, hydrogen iodide, sulfuric acid, phosphoric acid; and organic acids such as formic acid, acetic acid, propanoic acid, hydroxyacetic acid, lactic acid, pyruvic acid, glycolic acid, maleic acid, malonic acid, oxalic acid, benzenesulfonic acid, toluenesulfonic acid, methanesulfonic acid, trifluoroacetic acid, fumaric acid, succinic acid, malic acid, tartaric acid, citric acid, salicylic acid, p-aminosalicylic acid, pamoic acid, benzoic acid, ascorbic acid and the like. Exemplary base addition salt forms are the sodium, potassium, calcium salts, and salts with pharmaceutically acceptable amines such as, for example, ammonia, alkylamines, benzathine, and amino acids, such as, e.g. arginine and lysine. The term addition salt as used herein also comprises solvates which the compounds and salts thereof are able to form, such as, for example, hydrates, alcoholates and the like.

For clinical use, the compounds of the invention are formulated into pharmaceutical formulations for oral, rectal, parenteral or other mode of administration. Pharmaceutical formulations are usually prepared by mixing the active substance, or a pharmaceutically acceptable salt thereof, with conventional pharmaceutical excipients. Examples of excipients are water, gelatin, gum arabicum, lactose, microcrystalline cellulose, starch, sodium starch glycolate, calcium hydrogen phosphate, magnesium stearate, talcum, colloidal silicon dioxide, and the like. Such formulations may also contain other pharmacologically active agents, and conventional additives, such as stabilizers, wetting agents, emulsifiers, flavouring agents, buffers, and the like.

The formulations can be further prepared by known methods such as granulation, compression, microencapsulation, spray coating, etc. The formulations may be prepared by conventional methods in the dosage form of tablets, capsules, granules, powders, syrups, suspensions, suppositories or injections. Liquid formulations may be prepared by dissolving or suspending the active substance in water or other suitable vehicles. Tablets and granules may be coated in a conventional manner.

In a further aspect the invention relates to methods of making compounds of any of the formulae herein comprising reacting any one or more of the compounds of the formulae delineated herein, including any processes delineated herein. The compounds of Formula (I) above may be prepared by, or in analogy with, conventional methods.

The processes described above may be carried out to give a compound of the invention in the form of a free base or as an acid addition salt. A pharmaceutically acceptable acid addition salt may be obtained by dissolving the free base in a suitable organic solvent and treating the solution with an acid, in accordance with conventional procedures for preparing acid addition salts from base compounds. Examples of addition salt forming acids are mentioned above.

The compounds of Formula (I) may possess one or more chiral carbon atoms, and they may therefore be obtained in the form of optical isomers, e.g., as a pure enantiomer, or as a mixture of enantiomers (racemate) or as a mixture containing diastereomers. The separation of mixtures of optical isomers to obtain pure enantiomers is well known in the art and may, for example, be achieved by fractional crystallization of salts with optically active (chiral) acids or by chromatographic separation on chiral columns.

The chemicals used in the synthetic routes delineated herein may include, for example, solvents, reagents, catalysts, and protecting group and deprotecting group reagens. Examples of protecting groups are t-butoxycarbonyl (Boc), benzyl and trityl (triphenylmethyl). The methods described above may also additionally include steps, either before or after the steps described specifically herein, to add or remove suitable protecting groups in order to ultimately allow synthesis of the compounds. In addition, various synthetic steps may be performed in an alternate sequence or order to give the desired compounds. Synthetic chemistry transformations and protecting group methodologies (protection and deprotection) useful in synthesizing applicable compounds are known in the art and include, for example, those described in R. Larock, Comprehensive Organic Transformations, VCH Publishers (1989); T. W. Greene and P. G. M. Wuts, Protective Groups in Organic Synthesis, 3^{rd} Ed., John Wiley and Sons (1999); L. Fieser and M. Fieser, Fieser and Fieser's Reagents for Organic Synthesis, John Wiley and Sons (1994); and L. Paquette, ed., Encyclopedia of Reagents for Organic Synthesis, John Wiley and Sons (1995) and subsequent editions thereof.

The necessary starting materials for preparing the compounds of Formula (I) are either known or may be prepared in analogy with the preparation of known compounds. The dose level and frequency of dosage of the specific compound will vary depending on a variety of factors including the potency of the specific compound employed, the metabolic stability and length of action of that compound, the patient's age, body weight, general health, sex, diet, mode and time of administration, rate of excretion, drug combination, the severity of the condition to be treated, and the patient undergoing therapy. The daily dosage may, for example, range from about 0.001 mg to about 100 mg per kilo of body weight, administered singly or multiply in doses, e.g. from about 0.01 mg to about 25 mg each. Normally, such a dosage is given orally but parenteral administration may also be chosen.

The invention will now be further illustrated by the following non-limiting Examples. The specific examples below are to be construed as merely illustrative, and not limitative of the remainder of the disclosure in any way whatsoever. Without further elaboration, it is believed that one skilled in the art can, based on the description herein, utilize the present invention to its fullest extent. All publications cited herein are hereby incorporated by reference in their entirety.

### EXAMPLES

Experimental Methods

All reagents were commercial grade and were used as received without further purification, unless otherwise specified. The chemicals were bought from Sigma-aldrich (The old brickyard, New road, Gillingham, Dorset, SP8 4XT, UK), Lancaster (Eastgate, White Lund, Morecambe, Lancashire, LA3 3DY, UK), and Acros (Bishop Meadow road, Loughborough, leicestershire, LE11 5RG, UK). Commercially available anhydrous solvents were used for reactions conducted under inert atmosphere. Reagent grade solvents were used in all other cases, unless otherwise specified. Column chromatography was performed on Matrex® silica gel 60 (35-70 micron) or on Silica gel 60 (0.04-0.063 mm) (Merck. TLC was carried out using pre-coated silica gel F-254 plates (thickness 0.25 mm). ¹H NMR spectra were recorded on a Bruker Advance 250, on an Bruker Advance 400, on an Eclipse 270 (Jeol) walk-up instrument or on a Inova 400 (Varian) and Inova 500 (Varian). Chemical shifts for ¹H NMR spectra are given in part per million and either tetramethylsilane (0.00 ppm) or residual solvent peaks were used as internal reference. Splitting patterns are designated as follows: s, singlet; d, doublet; t, triplet; q, quartet; p, pentet; m, multiplet; br, broad. Coupling constants are given in Hertz (Hz). Only selected data are reported. The ¹³C NMR spectra were recorded at 62.5 MHz or 100.6 MHz. DEPT experiments were used to help assign ¹³C NMR resonances where necessary. Chemical shifts for ¹³C NMR spectra are expressed in parts per million and residual solvent peaks were used as internal reference. HPLC analyses were performed using a Waters Xterra MS C18 column (100×4.6 mm, 5μ) eluting with a gradient of 5% ACN in 95% water to 95% ACN in 5% water (0.2% TFA buffer) over 3.5 mins, then 95% ACN in 5% water (0.2% TFA buffer) for a further 2.5 mins at a flow rate of 3 mL/min on a Waters 600E or Gilson system with monitoring at 254 nm. HPLC was also run on HP1100 (Hewlett-Packard/Agilent) using System A: ACE 3 C8-column, 50×3 mm, System B: YMC ODS AQ-column 33×3 mm both run at 40° C. or System C: Hypersil 30×4.6 mm run at 25° C., all with 1 mL/min acetonitrile/water with 0.1% TFA as eluent. LC-MS was run on an LCD-MS (Agilent) with an HP1100 HPLC. MS was also run on LCZ (Micromass). Reverse phase preparative HPLC was carried out using a Xterra MS C18 column (100×19 mm, 5 μm) eluting with a gradient of 5% ACN in 95% water to 95% ACN in 5% water (0.05% DEA) over 12.0 mins, then 95% ACN in 5% water (0.05% DEA) for a further 5.0 min at a flow rate of 25 mL/min with monitoring at 254 nm. The fractions that contained the desired product were concentrated under reduced pressure and the resultant residue was lyophilised from a mixture of dioxane and water. Preparative HPLC was also performed on a Gilson system equipped with YMC ODS-AQ (150×30 mm) using water (containing 0.1% TFA)-acetonitrile gradient with a flow of 30 mL/min or a ACE 5 C8 column (30×150 mm), 40 mL/min, with different gradients of acetonitrile/water with 0.1% TFA as eluent with monitoring at 220 nm. Electrospray MS spectra were obtained on a Micromass platform LCMS spectrometer. Accurate mass measurements were performed on a Micromass LCT dual probe. The microwave heatings were made in a SmithCreator from Personal Chemistry. The optical rotations were obtained on a Perkin Elmer polarimeter 341 with a 100 mm cell at ambient temperature. Compounds were named using ACD/Name, version ACD/Labs 6.00 from Advanced Chemistry Development Inc. Capillary electrophoresis were performed on an Agilent CE with a fused silica tubing (400 mm×50 μm i.d.) using 10% HSC-μ in 25 mM phosphate buffer pH 2.5, voltage −15 kV, temperature 20° C. with the injection time 5 sec at 50 mbar. Analytical chiral LC was performed on a HP1100 using a Chirobiotic V column (250×4.6 mm) with the mobile phase MeOH/HOAc/TEA 100/0.5/0.5, 1.5 mL/min. Preparative chiral separation was performed on a Chirobiotic V2 column (250×21.2 mm), 30 mL/min. 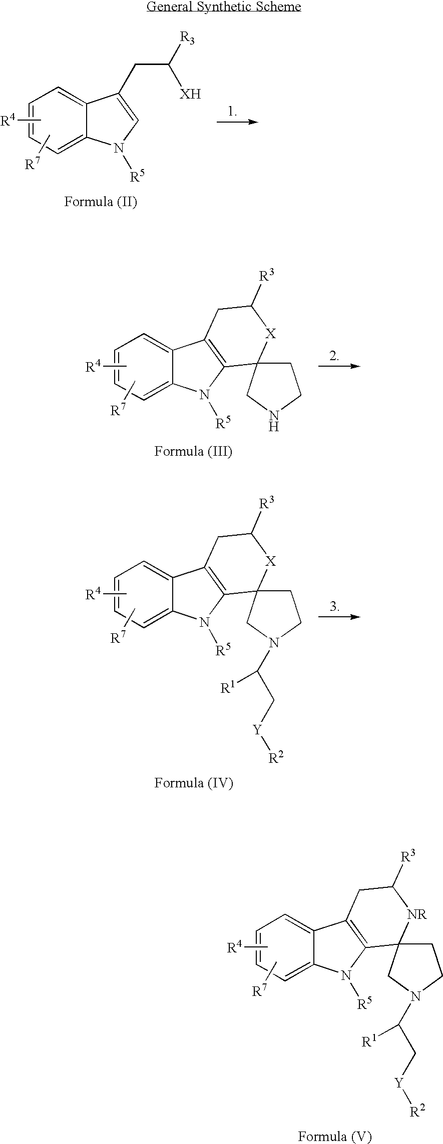
1.=a) N-Boc-3-pyrrolidinone b) HCl-dioxane, rt o/n; alt. a) N-benzyl-3-pyrrolidinone, b) Pd(OH)₂, H₂, 55° C.; alt. a) 1-(phenoxyethyl)pyrrolidin-3-one; AcOH, 100° C., 4 h
2.=R²—Y—CH₂—CH(R¹)-LG, TEA or dry K₂CO₃ in ACN/MeOH alt. DIPEA in DMSO, o/n, heating
3.=Ac₂O, DCM, o/n, 50° C. alt. isocyanate, DCM, rt alt. alkylating agent
   General Synthetic Procedure A
   Pictet Spengler. Method A.

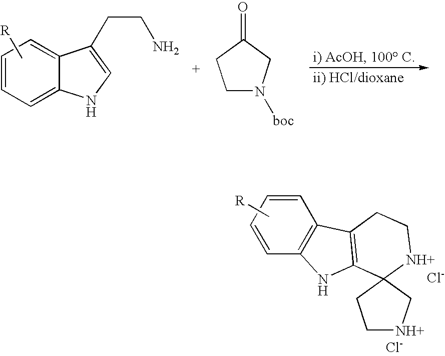

In 250 mL round bottom flask, tryptamines (as free-base or HCl salts) and N-Boc-3-pyrrolidinone (1.05 eq) were dissolved in AcOH (100-150 mL). Reactions were heated to 100° C. with constant stirring for 4-6 h under balloon N₂. Reaction mixture cooled to ambient and HCl (4.0M dioxan sol^{n.}) (5.0 eq) was added dropwise and stirred for 2-8 h. Filtered through No. 2 sintered glass funnel. Precipitate washed with several aliquots of Et₂O and dried to yield fine, dark brown to black powders.

Pictet Spengler. Method B.

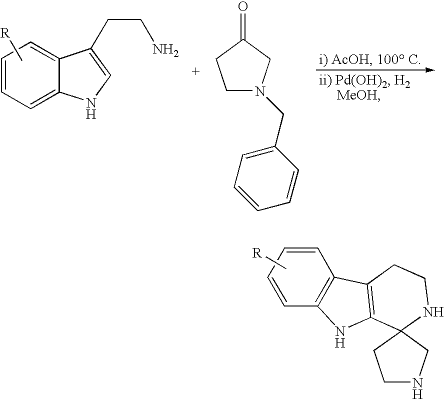

To a suspension of tryptamine hydrochloride (7.10 g, 31.3 mmol) in HOAc (50 mL) was added N-benzyl-3-pyrrolidinone (5.76 g, 32.9 mmol) and the mixture was heated at 100° C. for 1 h and 20 min. The solvent was removed at reduced pressure and the remaining oil was chromatographer on a column of silica initially with two column volumes of CHCl₃ 100% followed by CHCl₃/MeOH/aq conc NH₃ 95/5/0.2 to give 9.80 g (28.2 mmol, 90%) of a brown oil that crystallized upon standing. An analytical sample was precipitated as its hydrochloride salt with HCl/ether to give grey crystalline solid.

20% Pd(OH)₂ including 60% moisture (0.60 g) was added to the amine (4.35 g, 12.5 mmol) in MeOH (150 mL). The mixture was hydrogenated under stirring at 58° C. overnight. The reaction mixture was filtered through a pad if silica and the solvent was removed under reduced pressure to give 2.94 g (11.4 mmol, 91%) of a light brown foam that solidified in the evaporator. A small amount of the amine was nearly dissolved in warm MeOH and after cooling could white crystals be filtered off and dried (60° C., 10 mmHg) overnight.

Pictet Spengler. Method C.

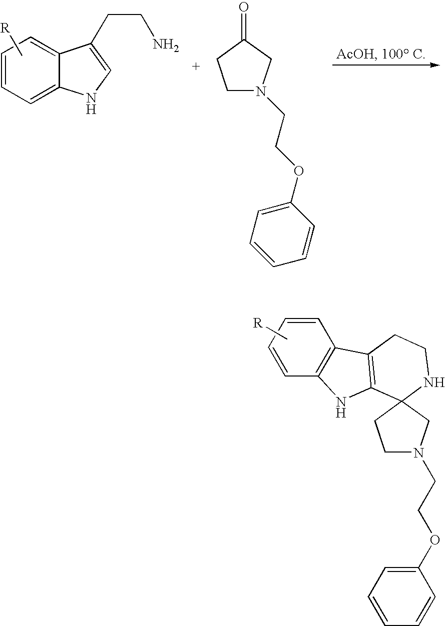

The tryptamine (0.16 mmol) and 1-(2-phenoxyethyl)pyrrolidin-3-one (0.16 mmol) were dissolved in HOAc (1 mL) and the mixture was heated at 100° C. for 40 min. The reaction mixture was then diluted with methanol (0.5 mL) and purified by direct injection to a preparative HPLC/UV System, MeCN:H₂O (0.1% TFA).

General Synthetic Procedure B

Alkylation. Method A.

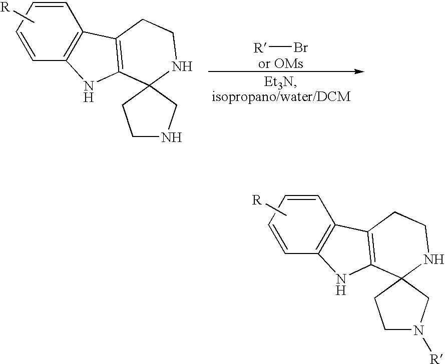

To a solution of carboline intermediate (0.2 mmol) and triethylamine (0.6 mmol) in isopropanol (0.9 mL) and water (0.1 mL) was added a solution of alkyl bromide or mesylate (0.2 mmol) in dichloromethane (0.5 mL). Aqueous 1M potassium carbonate solution (0.6 mL) was added and the reaction was heated at 80° C. for 18 h. The reaction was evaporated under reduced pressure. The crude product was then dissolved in DMSO (1 mL) and purified by preparative HPLC to give the desired product.

Alkylation. Method B.

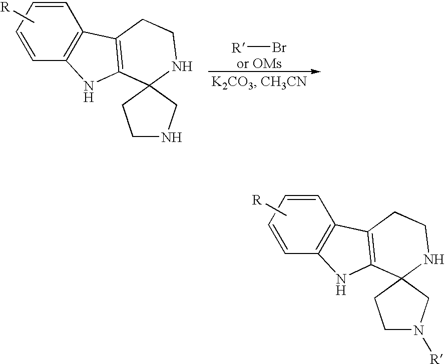

6-Methoxy-2,3,4,9-tetrahydrospiro[beta-carboline-1,3′-pyrrolidine] (0.26 mmol, 66 mg) was dissolved in DMF (1 mL). K₂CO₃ (0.156 mmol, 21 mg) and the alkylating agent (0.26 mmol) dissolved in 1 mL CH₃CN was added. The mixtures were agitated at 70° C. for 4 h-2 days. The samples were concentrated and purified by prep HPLC.

Alkylation. Method C.

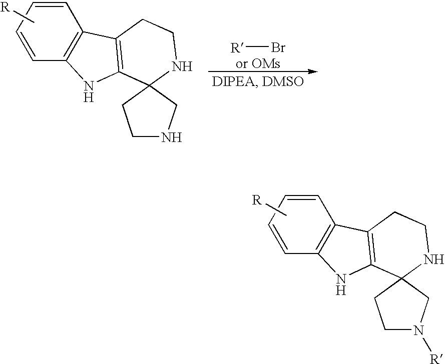

6-Methoxy-1′-(2-phenoxyethyl)-2,3,4,9-tetrahydrospiro[beta-carboline-1,3′-pyrrolidine] (1 eq) was mixed with alkylating agent (2.25 eq) and DIPEA (3 eq) in DMSO (250 uL) at 100° C. overnight. The reaction mixture was diluted with MeOH and TFA (50 uL) and purified with preparative HPLC (System A).

Alkylation. Method D.

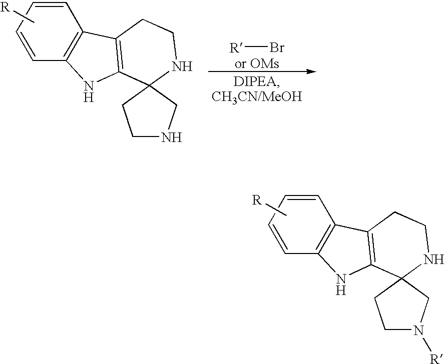

A solution of alkyl bromide (0.1 g, 0.88 mmol) in dry acetonitrile (2 mL) was added to a stirred mixture of pyrrolidine (0.44 mmol), N,N-diisopropylethylamine (0.19 mL, 1.09 mmol), dry acetonitrile (5 mL) and dry methanol (2.5 mL). The reaction mixture was heated to 80° C. for 22 h. The solvent was removed under reduced pressure and the residue dissolved in ethyl acetate. The solution was washed with water, dried over magnesium sulfate, filtered, and evaporated in vacuo. The resulting crude product was purified by flash chromatography eluting with a mixture of methanol and dichloromethane 1-99.

General Synthetic Procedure C.

Urea Formation.

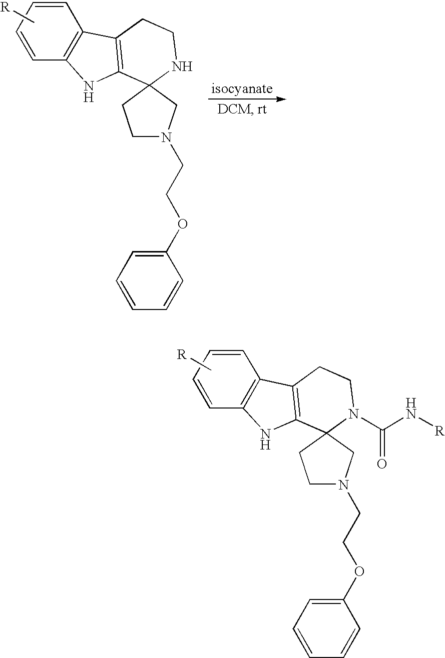

The alkylated beta-carbolines were dissolved in 500 uL dry DCM and treated with 1.25 eq of isocyanate. Mixing at rt. In some reactions, 500 CH₃CN was added to solubilize starting materials. More isocyanates were added after 3 h to those reactions that were in need. Evaporation, dissolution in MeOH (1 mL) and TFA (50 uL), filtration and purification by preparative HPLC (TFA).

General Synthetic Procedure D

Amide Formation. Method A.

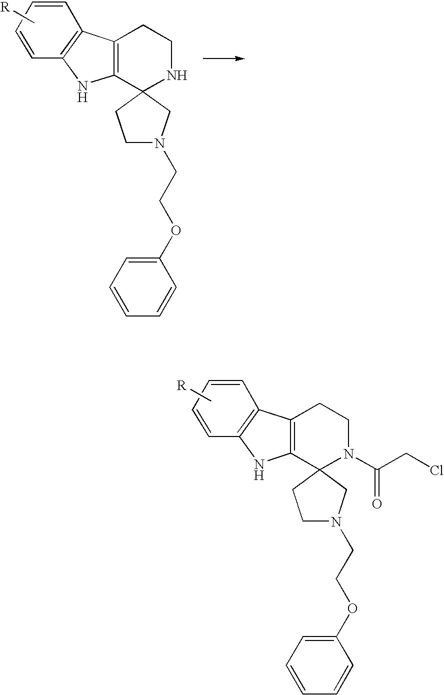

To a solution of 6-methoxy-1′-(2-phenoxyethyl)-2,3,4,9-tetrahydrospiro[beta-carboline-1,3′-pyrrolidine] (1.66 g, 4.4 mmol) in ACN (40 mL) was added K₂CO₃, under stirring was chloroacetyl chloride carefully added and the slightly warm mixture was stirred at ambient temperature for 10 minutes. The suspension was filtered through a two-layered pad, containing of silica (3 mm) and on top Celite (10 mm) finally was the solvent was removed at reduced pressure to give 1.78 g (3.92 mmol, 89%) of a yellow oil. The crude was used without further purification in the next synthetic step.

Amide Formation. Method B.

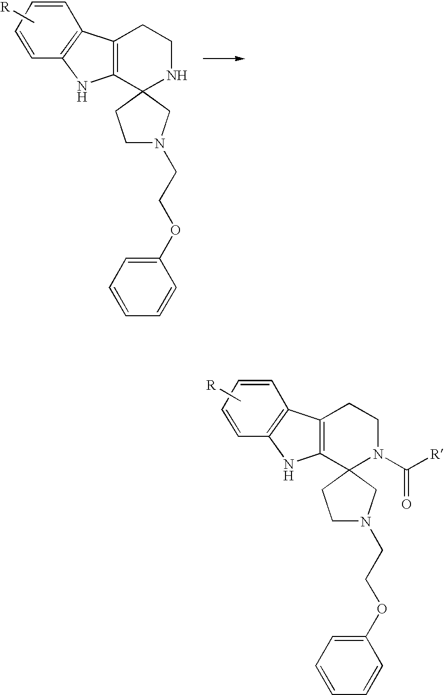

To a solution of the N-substituted [beta-carboline-1,3′-pyrrolidine] (0.05 mmol) in CH₃CN (1 mL), pyridine (0.15 mmol) and acetic anhydride (0.065 mmol, 6.64 mg) were added.

The mixtures were agitated for 2 days at 70° C. The samples were concentrated, dissolved in methanol and purified with preparative HPLC.

General Synthetic Procedure E

Alkylation. Method A.

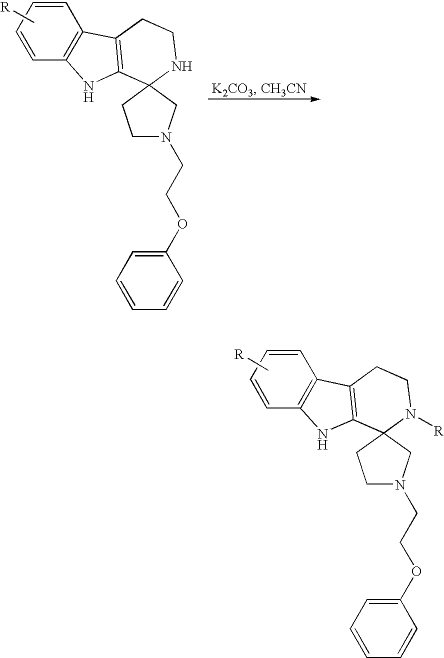

6-Methoxy-1′-(2-phenoxyethyl)-2,3,4,9-tetrahydrospiro[beta-carboline-1,3′-pyrrolidine] (1 eq) was mixed with alkylating agent (1.1 eq) and K₂CO₃ in dry CH₃CN (15 mL/mmol). The procedure was ineffective with acrylates or the corresponding 1-halopropionic ester/nitriles as alkylating agents. The solvent was evaporated, the residue dissolved in MeOH with TFA (50 uL) and purified with preparative HPLC (TFA).
Alkylation. Method B.

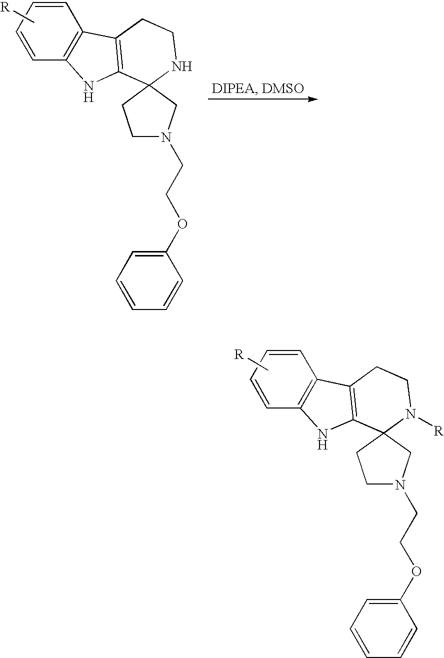

6-Methoxy-1′-(2-phenoxyethyl)-2,3,4,9-tetrahydrospiro[β-carboline-1,3′-pyrrolidine] (1 eq) was mixed with alkylating agent (2.25 eq) and DIPEA (3 eq) in DMSO (250 uL) at 100° C. overnight. The reaction mixture was diluted with MeOH and TFA (50 uL) and purified with preparative HPLC (System A).

General Synthetic Procedure F

Alkylation.

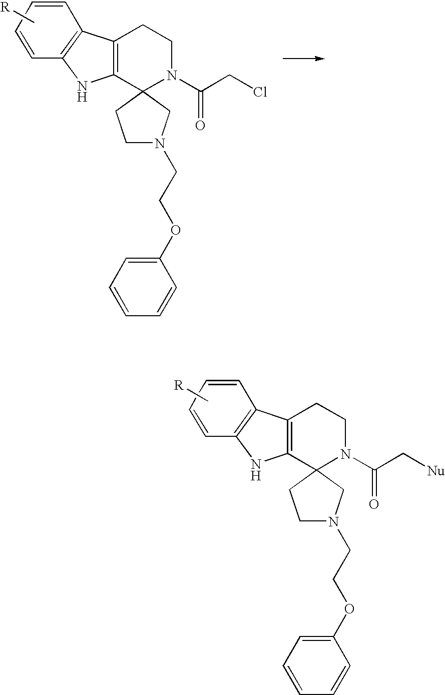

To the appropriate nucleophile (0.3 mmol) was added a solution of 2-(chloroacetyl)-6-methoxy-1′-(2-phenoxyethyl)-2,3,4,9-tetrahydrospiro[beta-carboline-1,3′-pyrrolidine], (25 mg, 55 μmol) in ACN (1 mL) and the mixtures were stirred at room temperature overnight. The reaction mixtures were filtered through a small pad of Celite, the solvent was removed at reduced pressure and the remaining residues were purified with preparative HPLC on an ACE C8-column with different gradients of acetonitrile/0.1% TFA with UV-detection. The pure fractions were combined and the solvent was removed at reduced pressure to give the target compounds as light brown oils.
General Synthetic Procedure G

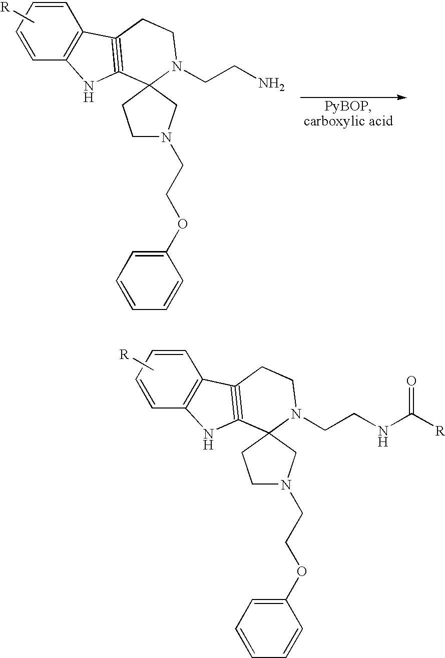

EXAMPLE 168 or EXAMPLE 157 (10.0 mg, 0.024 mmol) were weighed into screw-capped tubes and dissolved in DCM (300 uL). PyBOP (0.036 mmol) was added to each tube followed by a set of carboxylic acids (0.026 mmol). The mixtures were stirred for a couple of minutes and DIPEA (0.059 mmol) was added. The mixtures were heated in a stem-block at 50° C. overnight. The reaction mixtures were filtered and diluted with MeOH and purified by direct injection to a preparative HPLC/MS system, eluated with MilliQ water, MeCN and MilliQ/MeCN/0.1% TFA or MilliQ water, MeCN and NH₄HCO₃, or purified by reversed phase preparative HPLC using XTerra Prep MS C18 5 μm 19×50 mm, flow 25 mL/min, 50 mM pH10 NH₄HCO₃/ACN, fractions collected based on UV-signal (220 or 254 nm). Deprotection of the boc-group was performed on compounds containing a boc-protected amine, by using DCM/TFA (20%) and allowing them to stir at room temperature overnight. The solvent was then removed under reduced pressure.
General Synthetic Procedure H

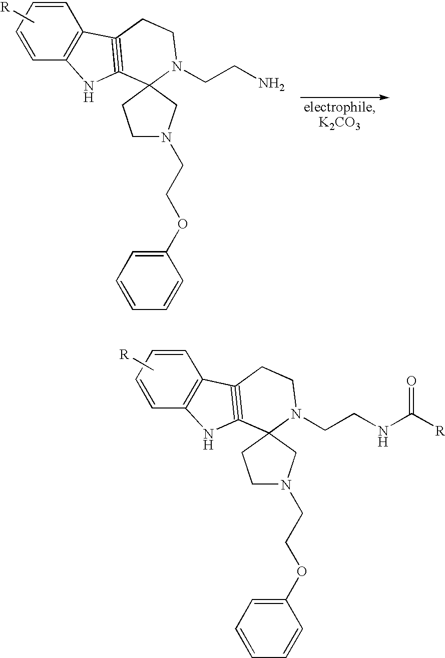

EXAMPLE 168 or EXAMPLE 157 (10.0 mg, 0.024 mmol were weighed into screw-capped tubes and dissolved in ACN (300 uL). K₂CO₃ (0.052 mmol) was added to each tube followed by a set of electrophiles (0.026 mmol). The mixtures were heated in a stem-block at 50° C. overnight. The reaction mixtures were filtered and diluted with MeOH and purified by direct injection to a preparative HPLC/MS system, eluated with MilliQ water, MeCN and MilliQ/MeCN/0.1% TFA or MilliQ water, MeCN and NH₄HCO₃.

Analysis

Routinely, post-synthesis all compounds are purified using reverse phase HPLC using a Gilson preparative HPLC System (322 pump, 155 UV/VIS detector, 215 liquid handler) and a Xterra MS, 100×19 mm, C18, 5 μm column. A flow rate of 25 mL/min is used.

The Gilson 215 acts as both auto-sampler and fraction collector

The gradient used is 90% water (0.05% DEA)/10% ACN for 1.5 min to 100% ACN over 5.5 min then held at 100% ACN for 3.0 min. The solvent mixture is then returned to the initial conditions over 0.5 min.

The purification is controlled by Unipoint software, triggering a threshold collection value monitoring at either 235 nm (PS203 and PS204) or 220 nm (PS205). Collected fractions are analysed by LCMS (Waters Alliance 2790 sampler with Micromass ZQ) (Table 1). The fractions that contain the desired product are concentrated by vacuum centrifugation and the resultant residue dried by freeze-drying.

TABLE 1

| Conditions | Detection |
|---|---|
| Column: Waters Xterra MS 5 μm | UV detection - diode |
| C18 100 × 4.6 mm | array range 210-350 nm. |
| Gradient: 95% water (10 mM NH₄HCO₃)/5% | Electrospray |
| ACN for 0.3 min then 95% water | ionisation: |
| (10 mM NH₄HCO₃)/5% ACN to | Cone voltage: 30 V. |
| 2% water (10 mM NH₄HCO₃)/98% ACN | Cone temperature: |
| over 4.0 min. Held at 2% water | 20° C. |
| (10 mM NH₄HCO₃)/98% ACN for 0.65 min. | Source temperature |
| The solvent mixture is then returned to the | 150° C. |
| initial conditions over 0.1 min and the | RF lens voltage: 0.0 V. |
| System allowed to re-equilibrate for 0.2 min. | Ion energy: 0.5 eV. |
| Flow rate: 2.0 mL/min. | Multiplier: 650 V. |
| Temperature: 30° C. | |
| Injection volume: 5 μL partial loop. | |

### Comparative Example 1

### 6-Methoxy-2,3,4,9-tetrahydrospiro[beta-carboline-1,3′-pyrrolidine]

5-Methoxytryptamine hydrochloride (227 mg, 1.0 mmol) and 1-benzyl-3-pyrrolidinone (184 mg, 1.05 mmol) and acetic acid (5 mL) was heated to 100° C. for 1 h. The solution was cooled, 25 mg 10% Pd/C and abs. EtOH (5 mL) was added. Debenzylation was made by hydrogenation for 3 days. The catalyst was filtered off and the solution was evaporated. 2-Propanol (3×5 mL) was added and evaporated. The residue was partially solid. Diethyl ether and MeOH was added and evaporated. The resulting product (381 mg) was shown by NMR to contain acetic acid. The free base was obtained by adding 2M Na₂CO₃ (2 mL) and extracting with CHCl₃. After drying (Na₂SO₄) and evaporation of the solvent the title compound (254 mg, 99%) was obtained as a beige solid.

HPLC ca 90%, R_{T}=1.12 min (System A, 10-97% MeCN over 3 min).

¹H NMR (270 MHz, DMSO-d6) δ 1.70-1.87 (m, 1H), 1.94-2.10 (m, 1H), 2.73-3.09 (m, 7H), 3.72 (s, 3H), 6.62 (dd, J=2.4, 8.6 Hz, 1H), 6.82 (d, J=2.2 Hz), 7.13 (d, J=8.7 Hz, 1H), 10.48 (s, 1H).

MS (ESI+) m/z 258 (M+H)⁺.

alt.

To a solution of the starting amine (EXAMPLE 126, 4.35 g, 12.5 mmol) in MeOH (150 mL) was added 20% Pd(OH)₂ including 60% moisture (0.60 g) and the mixture was vigorously stirred at 58° C. overnight. The reaction mixture was filtered through a pad if silica and the solvent was removed under reduced pressure to give 2.94 g (11.4 mmol, 91%) of a light brown foam that solidified in the evaporator. A small amount of the amine was nearly dissolved in warm MeOH and after cooling could white crystals be filtered off and dried (60° C., 10 mmHg) overnight.

M. p. >180° C. (at 270° C. there was a black foam in the melting glasstube)

HPLC 100%, R_{T}=1.24 min (System A. 5-60% MeCN), 100%, R_{T}=0.991 min (System B. 2-20% MeCN). Attention! System B gradient 1 min 30 sec.

1H NMR (400 MHz, DMSO-D6) δ ppm 2.64-2.79 (m, J=8.53 Hz, 2H) 2.85-2.99 (m, 2H) 3.37 (s, 2H) 3.50 (t, J=5.14 Hz, 2H) 3.62-3.80 (m, 6H) 3.97 (d, J=13.55 Hz, 1H) 6.79 (dd, J=8.78, 2.26 Hz, 1H) 6.98 (d, J=2.26 Hz, 1H) 7.25 (d, J=8.78 Hz, 1H) 11.49 (s, 1H)

¹³C NMR (CDCl₃) δ 18.17, 35.56, 44.59, 48.75, 52.58, 55.68, 63.18, 100.53, 108.08, 112.62, 113.08, 125.96, 128.93, 131.48, 153.88.

MS (ESI+) m/z 258 (M+H)⁺.

HRMS (EI) calcd for C₁₅H₁₉N₃O: 257.1528, found 257.1535.

### Comparative Example 2

### 2,3,4,9-Tetrahydrospiro[beta-carboline-1,3′-pyrrolidine]

To a 250 mL 3-necked round bottom flask was added tryptamine (3.2 g, 20 mmol) and N-Boc-3-pyrrolidinone (3.9 g, 21 mmol) in AcOH (100 mL). The reaction was heated to 100° C. for 4.5 h under a nitrogen atmosphere. The reaction was cooled down to room temperature and HCl 4M in dioxane (25 mL, 0.1 mol) was added dropwise and the solution stirred at room temperature overnight. The solution was filtered through a sintered funnel and the solid washed with a small amount of diethyl ether. The solid was then dried to afford 7.54 g of crude product in quantitative yield. The amine was used in the next step without further purification.

HPLC 81%, Rt=1.36 min.

¹H-NMR (250 MHz, DMSO-d₆) δ 2.48 (q, 1H, J 1.7 Hz, CH), 2.75 (t, 2H, J=8.7 Hz, CH₂), 2.95 (bs, 2H, CH₂), 3.52-3.54 (m, 2H, CH₂), 3.70-3.98 (m, 3H, CH₂+CH), 7.03 (dt, 1H, J 0.7 Hz, J 7.8 Hz, Haro), 7.15 (dt, 1H, J 1.0/7.5 Hz, Haro), 7.35 (d, 1H, J 8 Hz, Haro), 7.47 (d, 1H, J 7.7 Hz, Haro), 9.75 (bs, 1H, NH), 10.42 (bs, 1H, NH), 10.70 (bs, 2H, NH₃⁺).

MS (AP) m/z 228 (M+H)⁺.

### Comparative Example 3

### 8-Methyl-2,3,4,9-tetrahydrospiro[beta-carboline-1,3′-pyrrolidine]

To a 250 mL 3-necked round bottom flask was added 7-methyltryptamine (3.2 g, 20 mmol) and N-Boc-3-pyrrolidinone (3.9 g, 21 mmol) in AcOH (100 mL). The reaction was heated to 100° C. for 4.5 h under a nitrogen atmosphere. The reaction was cooled down to room temperature and HCl 4M in dioxane (25 mL, 0.1 mol) was added dropwise and the solution stirred at room temperature overnight. The solution was filtered through a sintered funnel and the solid washed with a small amount of diethyl ether. The solid was then dried to afford crude product in quantitative yield. The amine was used in the next step without further purification.

HPLC 94%, Rt=1.67 min.

¹H-NMR (250 MHz, DMSO-d₆) δ 2.48 (q, 1H, J 1.7 Hz, CH), 2.75 (t, 2H, J=8.7 Hz, CH₂), 2.95 (brs, 2H, CH₂), 3.47 (s, 3H, Me), 3.52-3.54 (m, 2H, CH₂), 3.70-3.98 (m, 3H, CH₂+CH), 7.15 (dt, 1H, J 1.2 Hz, J=7.5 Hz, Harom), 7.35 (d, 1H, J 8 Hz, Harom), 7.47 (d, 1H, J 7.7 Hz, Harom), 9.75 (brs, 1H, NH), 10.42 (brs, 1H, NH).

MS (AP) m/z 242 (M+H)⁺.

### Example 4

### 6-Methoxy-1′-[2-(5-methyl-1H-indol-3-yl)ethyl]-2,3,4,9-tetrahydrospiro[beta-carboline-1,3′-pyrrolidine]

To a stirred solution of 6-methoxy-2,3,4,9-tetrahydrospiro[beta-carboline-1,3′-pyrrolidine] (COMPARATIVE EXAMPLE 1, 135 mg, 0.52 mmol) in dry ACN (2 mL) and MeOH (1 mL) was added triethylamine (74 μL, 0.52 mmol) and methanesulfonic acid-2-(5-methyl-1H-indol-3-yl)-ethyl ester (96 mg, 0.35 mmol) and the reaction mixture was stirred for 17 h at 55° C. The mixture was evaporated and the resulting crude oil was extracted with ethyl acetate, washed with 2N NaOH, dried over magnesium sulfate then concentrated in vacuo. The desired amine was purified by flash chromatography on silica gel eluting with a gradient of ethyl acetate/methanol (9:1 to 7:3) and afforded the desired compound as a pale brown solid (28.8 mg, 20%).

HPLC 100%, Rt=1.95 min.

¹H-NMR (250 MHz, CDCl₃) δ 1.98-2.35 (m, 4H, CH₂), 2.45 (s, 3H, CH₃), 2.59-2.70 (m, 3H, CH), 2.91-3.32 (m, 7H, CH₂), 3.84 (s, 3H, —OMe), 6.74-6.78 (dd, 1H, J 2.4/4.9 Hz, Harom), 6.89-6.97 (m, 4H, Harom), 7.04-7.08 (dd, 2H, J 1.3/8.2 Hz, Harom), 7.32-7.41 (t, 2H, J 15.7/24 Hz, Harom), 7.98 (brs, 1H, NHindole), 8.73 (brs, 1H, —NH).

MS (AP) m/z 415 (M+H)⁺.

### Example 5

### N-Cyclohexyl-6-methoxy-1′-(2-phenoxyethyl)-4,9-dihydrospiro[beta-carboline-1,3′-pyrrolidine]-2(3H)-carboxamide

Prepared in the same way as EXAMPLE 27. Yield 70 mg.

HPLC 99%, R_{T}=2.46 min (System A, 10-97% MeCN over 3 min).

¹H NMR (270 MHz, CDCl₃) δ 1.01-1.23 (m, 3H), 1.24-1.44 (m, 2H), 1.51-1.77 (m, 3H), 1.83-2.05 (m, 2H), 2.19-2.36 (m, 1H), 2.57-3.11 (m, 8H), 3.16 (d, J=9.4 Hz, 1H), 3.41 (d, J=9.4 Hz, 1H), 3.55-3.74 (m, 2H), 3.76-3.90 (m, 4H), 4.09 (t, J=5.2 Hz, 2H), 6.09 (d, J=7.2 Hz, 1H), 6.78 (dd, J=2.5, 8.7 Hz, 1H), 6.87-7.01 (m, 4H), 7.15 (d, J=8.9 Hz, 7.26-7.35 (m, 2H), 9.01 (s, 1H).

MS (ESI+) m/z 503 (M+H)⁺.

### Example 6

### 2-Acetyl-1′-[2-(4-fluorophenoxy)ethyl]-6-methoxy-2,3,4,9-tetrahydrospiro[beta-carboline-1,3′-pyrrolidine]

To a stirred solution of EXAMPLE 69 (54 mg, 0.137 mmol) in dry DCM (2 mL) was added triethylamine (48 μL, 0.342 mmol). To the clear solution was added acetic anhydride (33 μL, 0.342 mmol) and the reaction mixture was stirred for 17 h at 50° C. The reaction mixture was evaporated and the resulting crude oil was extracted with ethyl acetate, washed with water, dried over magnesium sulfate then concentrated in vacuo. The desired amine was obtained as a pale brown solid (40.3 mg, 67%).

HPLC 95%, Rt=2.20 min.

¹H-NMR (250 MHz, CDCl₃) δ 1.94 (brs, 2H, CH₂), 2.26 (s, 3H, CH₃), 2.68 (m, 3H, CH), 2.82 (t, 2H, J=5.4 Hz, CH₂—O), 2.99-3.66 (m, 7H, CH₂), 3.84 (s, 3H, —OMe), 4.13 (brs, 2H, CH—N), 6.77-7.02 (m, 6H, Harom), 7.14 (d, 1H, J 7.8 Hz, Harom), 9.52 (brs, 1H, NH).

MS (AP) m/z 438 (M+H)⁺.

### Example 7

### 2-Acetyl-6-methoxy-1′-(2-phenoxyethyl)-2,3,4,9-tetrahydrospiro[beta-carboline-1,3′-pyrrolidine]

The title compound was prepared from EXAMPLE 10 (0.05 mmol) as described in the General Synthetic Procedure D, Method B to afford 0.0278 g.

HPLC 100%, Rt=1.278 min.

¹H-NMR (250 MHz, DMSO-d₆) δ 7.22-7.17 (m, 3H), 6.93-6.87 (m, 4H), 6.77-6.68 (m, 1H), 4.30-3.91 (m, 5H), 3.70 (s, 3H), 3.75-3.47 (m, 5H), 2.74-2.49 (m, 4H), 2.19 (s, 3H).

MS (ESI+) m/z 420 (M+H)⁺.

### Example 8

### 1′-(2-Cyclohexylethyl)-6-methoxy-2,3,4,9-tetrahydrospiro[beta-carboline-1,3′-pyrrolidine]

6-Methoxy-2,3,4,9-tetrahydrospiro[beta-carboline-1,3′-pyrrolidine] (COMPARATIVE EXAMPLE 1, 80 mg, 0.31 mmol) and 1-bromo-2-cyclohexylethane (65 mg, 0.34 mmol) was dissolved in CH₃CN-DMF 1:1 (3 mL). K₂CO₃ (24 mg, 0.17 mmol) was added and the mixture was heated to 70° C. for 2 h. The solvent was evaporated and the residue was purified by preparatory HPLC (20-35% CH₃CN) affording the title compound as a TFA-salt. (44 mg, 30%).

HPLC 100%, R_{T}=1.72 min (System A, 10-97% MeCN over 3 min).

¹H NMR (270 MHz, CD₃OD) δ 0.89-1.11 (m, 2H), 1.15-1.52 (m, 4H), 1.57-1.84 (m, 7H), 2.61-2.77 (m, 1H), 2.78-2.94 (m, 1H), 3.04 (t, J=6.4 Hz, 2H), 3.33 (m, obscured by solvent), 3.50-3.75 (m, 4H), 3.81 (s, 3H), 4.05-4.24 (m, 1H), 6.86 (dd, J=2.5, 8.9 Hz, 1H), 6.99 (d, J=2.5 Hz, 1H), 7.28 (d, J=8.9 Hz, 1H).

MS (ESI+) m/z 368 (M+H)⁺.

### Example 9

### 1′-(2-Phenoxyethyl)-2,3,4,9-tetrahydrospiro[beta-carboline-1,3′-pyrrolidine]

To a stirred solution of 2,3,4,9-tetrahydrospiro[beta-carboline-1,3′-pyrrolidine] (COMPARATIVE EXAMPLE 2, 100 mg, 0.44 mmol) in dry ACN (4 mL) and dry MeOH (2 mL) was added Hunig's base (79.5 μL, 0.66 mmol). To the clear solution was added β-bromophenetole (59 mg, 0.3 mmol) and the reaction mixture was stirred at 80° C. overnight. The reaction mixture was evaporated and the resulting crude oil was extracted with EtOAc, washed with water, 2N NaOH, dried over magnesium sulfate then concentrated in vacuo. The crude amine was then purified by flash chromatography on silica gel eluting with a mixture of methanol:EtOAc (3:7) and afforded the required product as a white solid (26.7 mg, 18%).

HPLC 99%, Rt=1.80 min.

¹H-NMR (250 MHz, MeOD) δ 2.05-2.15 (m, 1H, CH), 2.30-2.42 (m, 4H, CH₂), 2.58 (d, 1H, J 9.0 Hz, CH), 2.70-2.77 (m, 3H, —CH), 2.98-3.35 (m, 6H, —CH₂), 4.16 (t, 2H, J 5.4 Hz, CH—N), 6.93-7.16 (m, 5H, Harom), 7.28-7.34 (m, 3H, Harom), 7.45 (t, 1H, J 7.0 Hz, Harom), 9.2 (s, 1H, NH).

MS (AP) m/z 348 (M+H)⁺.

### Example 10

### 6-Methoxy-1′-(2-phenoxyethyl)-2,3,4,9-tetrahydrospiro[beta-carboline-1,3′-pyrrolidine]

COMPARATIVE EXAMPLE 1 (248 mg, 0.96 mmol), β-bromophenetole (213 mg, 1.06 mmol) and K₂CO₃ (73 mg, 0.53 mmol) was mixed with acetonitrile (5 mL) and DMF (5 mL). The mixture was heated to 70° C. for 2 h. The solvent was distilled off in vacuum and ⅓ of the crude product was purified by preparatory HPLC to give 66 mg of the title compound as a TFA salt. The remainder of the crude product was purified in the same way to give another batch (177 mg).

HPLC 100%, R_{T}=1.42 min (System A, 10-97% MeCN over 3 min).

¹H NMR (270 MHz, CD₃OD) δ 2.55-2.81 (m, 2H), 3.05 (t. J=5.9 Hz, 2H), 3.39-3.67 (m, 6H), 3.68-3.78 (m, 1H), 3.81 (s, 3H), 3.96 (d, J=12.6 Hz, 1H), 4.32 (t, J=5.1 Hz, 2H), 6.85 (dd, J=2.5, 8.9 Hz, 1H), 6.91-7.02 (m, 4H), 7.23-7.34 (m, 3H).

MS (ESI+) m/z 378 (M+H)⁺.

### Example 11

### 7-Fluoro-1′-(2-phenoxyethyl)-2,3,4,9-tetrahydrospiro[beta-carboline-1,3′-pyrrolidine]

The title compound was prepared according to General Synthetic Procedure A, Method C. Preparative HPLC/UV system, MeCN:H₂O (0.1% TFA) 18-39% afforded 34.8 mg (78%) white powder.

HPLC 100%, R_{T}=1.66 min (System A. 10-97% MeCN over 3 min), 100%, R_{T}=1.46 min (System B. 10-97% MeCN over 3 min).

¹H NMR (400 MHz, MeOD) δ ppm 2.54-2.68 (m, 2H) 3.05 (t, J=5.9 Hz, 2H) 3.40-385 (m, 8H) 4.27-4.30 (m, 2H) 6.84-6.90 (m, 1H) 6.93-6.97 (m, 3H) 7.08 (d, J=9.7 Hz, 1H) 7.26-7.30 (m, 2H) 7.46 (dd, J=8.6, 5.1 Hz, 1H).

MS (ESI+) m/z 366 (M+H)⁺.

### Example 12

### 6-Methoxy-2-(methylsulfonyl)-1′-(2-phenoxyethyl)-2,3,4,9-tetrahydrospiro[beta-carboline-1,3′-pyrrolidine]

EXAMPLE 10 (50 mg, 0.13 mmol) and Et₃N (16 mg, 0.16 mmol) was dissolved in DCM (1 mL). MsCl (25 mg, 0.16 mmol) was added and the solution was left for 4-h, then acidified with TFA and evaporated. The residue was purified by preparative HPLC (20-40% MeCN) affording 48 mg of the title compound as a TFA-salt.

HPLC 100%, R_{T}=1.94 min (System A, 10-97% MeCN over 3 min).

¹H NMR (270 MHz, CD₃OD) δ 2.73-2.94 (m, 3H), 2.95-3.12 (m, 4H), 3.70-4.28 (m, 10H), 4.39 (t, J=4.7 Hz, 2H, 4.53 (d, J=13.1 Hz, 1H), 6.77-6.87 (m, 1H), 6.92-7.07 (m, 4H), 7.21-7.37 (m, 3H).

MS (ESI+) m/z 456 (M+H)⁺.

### Example 13

### 1′-[2-(1,3-Benzodioxol-5-yl)ethyl]-6-methoxy-2,3,4,9-tetrahydrospiro[beta-carboline-1,3′-pyrrolidine]

The title compound was prepared according to General Synthetic Procedure A, Method A followed by General Synthetic Procedure B, Method A.

HPLC 96.8%, Rt=3.65 min.

MS (ESI+) m/z 406 (M+H)⁺.

### Example 14

### 6-Methoxy-1′-[2-(3-methoxyphenoxy)ethyl]-2,3,4,9-tetrahydrospiro[beta-carboline-1,3′-pyrrolidine]

The title compound was prepared from COMPARATIVE EXAMPLE 1 (66 mg, 0.26 mmol) and 2-(3-methoxyphenoxy)ethyl methanesulfonate (80 mg) as described in General Synthetic Procedure B, Method B to afford 0.0202 g.

HPLC 100%, Rt=1.492 min.

¹H-NMR (270 MHz, DMSO-d₆) δ 2.35-3.53 (m, 12H), 3.69-3.77 (d, 6H, J=14.13), 4.19-4.25 (s, 2H), 6.51-6.59 (m, 3H), 6.77-6.83 (m, 1H), 6.97-6.99 (m, 1H), 7.18-7.23 (m, 1H), 7.27-7.31 (d, 1H, J=8.80 Hz).

MS (ESI+) m/z 408 (M+H)⁺.

### Example 15

### 1′-[2-(2-Fluorophenoxy)ethyl]-6-methoxy-2,3,4,9-tetrahydrospiro[beta-carboline-1,3′-pyrrolidine]

The title compound was prepared from COMPARATIVE EXAMPLE 1 (66 mg, 0.26 mmol) and 2-(2-fluorophenoxy)ethyl methanesulfonate (66 mg) as described in General Synthetic Procedure B, Method B to afford 0.0288 g.

HPLC 97%, Rt=1.441 min.

¹H-NMR (270 MHz, DMSO-d₆) δ=2.32-3.17 (m, 12H), 3.72-3.79 (s, 3H), 4.25-4.32 (s, 2H), 6.76-6.80 (d, 1H, J=8.53 Hz), 6.93-6.99 (s, 2H), 7.11-7.29 (m, 4H), 10.92-10.95 (s, 1H).

MS (ESI+) m/z 396 (M+H)⁺.

### Example 16

### 6-Methoxy-1′-[2-(2-methoxyphenoxy)ethyl]-2,3,4,9-tetrahydrospiro[beta-carboline-1,3′-pyrrolidine]

The title compound was prepared from COMPARATIVE EXAMPLE 1 (66 mg, 0.26 mmol) and 2-(4-methoxyphenoxy)ethyl methanesulfonate (69 mg) as described in General Synthetic Procedure B, Method B to afford 0.0143 g.

HPLC 100%, Rt=1.465 min.

¹H-NMR (270 MHz, DMSO-d₆) δ 2.37-3.50 (m, 12H), 3.65-3.81 (d, 6H), 4.10-4.22 (s, 2H), 6.77-6.82 (d, 1H, J=9.14 Hz), 6.85-6.94 (m, 4H), 6.96-7.00 (s, 1H), 7.26-7.31 (d, 1H, J=8.52).

MS (ESI+) m/z 408 (M+H)⁺.

### Example 17

### 1′-[2-(4-Chlorophenoxy)ethyl]-6-methoxy-2,3,4,9-tetrahydrospiro[beta-carboline-1,3′-pyrrolidine]

The title compound was prepared from COMPARATIVE EXAMPLE 1 (66 mg, 0.26 mmol) and 2-(4-chlorophenoxy)ethyl methanesulfonate (71 mg) as described in General Synthetic Procedure B, Method B to afford 0.0172 g.

HPLC 100%, Rt=1.611 min.

¹H-NMR (270 MHz, DMSO-d₆) δ 2.34-3.52 (m, 12H), 3.72-3.81 (s, 3H), 4.17-4.26 (s, 2H), 6.77-6.82 (d, 1H, J=8.53 Hz), 6.96-7.04 (m, 3H), 7.26-7.31 (d, 1H, J=8.53 Hz), 7.32-7.37 (d, 2H, J=8.53 Hz).

MS (ESI+) m/z 412 (M+H)⁺.

### Example 18

### 6-Methoxy-1′-(1-methyl-2-phenoxyethyl)-2,3,4,9-tetrahydrospiro[beta-carboline-1,3′-pyrrolidine]

The title compound was prepared from COMPARATIVE EXAMPLE 1 (66 mg, 0.26 mmol) and 1-methyl-2-phenoxyethyl methanesulfonate (65 mg) as described in General Synthetic Procedure B, Method B to afford 0.0206 g.

HPLC 100%, Rt=1.497 min.

¹H-NMR (270 MHz, DMSO-d₆) δ 1.22-1.39 (s, 3H), 2.39-3.21 (m, 11H), 3.72-3.82 (s, 3H), 4.03-4.22 (s, 1H), 6.76-6.83 (d, 1H, J=7.31 Hz), 6.94-7.04 (m, 4H), 7.25-7.38 (m, 3H).

MS (ESI+) m/z 392 (M+H)⁺.

### Example 19

### 4-[2-(6-Methoxy-2,3,4,9-tetrahydro-1′H-spiro[beta-carboline-1,3′-pyrrolidin]-1′-yl)ethoxy]benzonitrile

The title compound was prepared from COMPARATIVE EXAMPLE 1 (66 mg, 0.26 mmol) and 2-(4-cyanophenoxy)ethyl methanesulfonate (68 mg) as described in General Synthetic Procedure B, Method B to afford 0.0287 g.

HPLC 100%, Rt=1.430 min.

¹H-NMR (270 MHz, DMSO-d₆) δ 2.34-3.51 (m, 12H), 3.71-3.80 (s, 3H), 4.26-4.37 (s, 2H), 6.76-6.84 (d, 1H, J=9.14 Hz), 6.95-7.01 (s, 1H), 7.11-7.19 (d, 2H, J=8.52 Hz), 7.25-7.31 (d, 1H, J=8.52 Hz), 7.76-7.83 (d, 2H, J=8.53 Hz).

MS (ESI+) m/z 403 (M+H)⁺.

### Example 20

### 2-Acetyl-6-methoxy-1′-[2-(2-methoxyphenoxy)ethyl]-2,3,4,9-tetrahydrospiro[beta-carboline-1,3′-pyrrolidine]

The title compound was prepared from EXAMPLE 223 (0.05 mmol) as described in General Synthetic Procedure D, Method B to afford 0.0070 g.

HPLC 100%, Rt=1.235 min.

¹H-NMR (270 MHz, DMSO-d₆) δ 2.26-2.30 (s, 3H), 2.40-4.34 (m, 12H), 3.67-3.70 (s, 3H), 3.75-3.78 (s, 3H), 4.35-4.43 (s, 2H), 6.78-6.82 (d, 1H, J=8.53 Hz), 6.88-6.94 (m, 1H), 6.98-7.02 (m, 2H), 7.06-7.10 (d, 1H, J=7.91 Hz), 7.27-7.31 (d, 1H, J=8.53 HZ).

MS (ESI+) m/z 450 (M+H)⁺.

### Example 21

### 2-Acetyl-1′-[2-(2-fluorophenoxy)ethyl]-6-methoxy-2,3,4,9-tetrahydrospiro[beta-carboline-1,3′-pyrrolidine]

The title compound was prepared from EXAMPLE 15 (0.05 mmol) as described in General Synthetic Procedure D, Method B to afford 0.0075 g.

HPLC 100%, Rt=1.288 min.

¹H-NMR (270 MHz, DMSO-d₆) δ=2.23-2.29 (m, 3H), 2.41-4.51 (m, 14H), 3.72-3.83 (s, 3H), 6.78-6.83 (d, 1H, J=9.13 Hz), 6.98-7.04 (s, 2H), 7.13-7.32 (m, 4H).

MS (ESI+) m/z 438 (M+H)⁺.

### Comparative Example 24

### 4,9-Dihydro-3H-spiro[pyrano[3,4-b]indole-1,3′-pyrrolidine]

To a solution of tryptophol (2.0 g, 12.4 mmol) in dry THF (50 mL) was added N-Boc-3-pyrrolidinone (2.33 g, 12.6 mmol) followed by boron trifluoride diethyl etherate (3.67 mL, 28.96 mmol). The mixture was stirred for 16 h at room temperature and 4 h at 80° C. under a nitrogen atmosphere. The reaction mixture was poured into 1M sodium hydroxide solution and extracted with ethyl acetate. The organic phase was dried over magnesium sulfate, filtered and evaporated under reduced pressure to give a brown oil. The residue was purified by flash chromatography eluting with a mixture of methanol and dichloromethane 1:4 and then 2:3 to give the product as a pink solid (1.27 g, 45%).

HPLC 100%, Rt=1.79 min.

¹H-NMR (250 MHz, CDCl₃) δ 2.11-2.30 (m, 2H), 2.83 (t, 2H, J=5.4 Hz), 3.11-3.37 (m, 5H), 3.99 (t, 2H, J 5.4 Hz), 7.07-7.19 (m, 2H), 7.34 (d, 1H, J 7.3 Hz), 7.50 (d, 1H, J 7.0 Hz), 9.33 (brs, 1H, NH). ¹³C-NMR (62.5 MHz, CDCl₃) δ=22.27 (CH₂), 38.99 (CH₂), 46.11 (CH₂), 58.17 (CH₂), 62.01 (CH₂), 81.88 (C_{q}), 107.83 (C_{q}), 111.07 (CH), 118.08 (CH), 119.39 (CH), 121.72 (CH), 126.72 (C_{q}), 135.11 (C_{q}), 136.13 (C_{q}).

MS (AP) m/z 229 (M+H)⁺.

### Example 25

### 1′-[2-(4-Fluorophenoxy)ethyl]-4,9-dihydro-3H-spiro[pyrano[3,4-b]indole-1,3′-pyrrolidine]

The title compound was prepared from COMPARATIVE EXAMPLE 24 using General Synthetic Procedure B, Method D. After the usual work-up, the product was isolated as a light brown oil (147.7 mg, 92%).

HPLC 100%, Rt=2.24 min.

¹H-NMR (250 MHz, CDCl₃) δ 2.16-2.38 (m, 2H), 2.64-2.77 (m, 3H), 2.82-3.05 (m, 3H), 3.17-3.26 (m, 2H), 3.90-4.00 (m, 1H), 4.05-4.13 (m, 3H), 6.81-6.90 (m, 2H), 6.93-6.98 (m, 2H), 7.00-7.17 (m, 2H), 7.24-7.29 (m, 1H), 7.48 (dd, 1H, J=0.98 Hz, 7.6 Hz), 8.83 (brs, 1H).

MS (AP) m/z 367 (M+H)⁺.

### Example 26

### 1′-(2-Phenoxyethyl)-4,9-dihydro-3H-spiro[pyrano[3,4-b]indole-1,3′-pyrrolidine]

The title compound was prepared from COMPARATIVE EXAMPLE 24 using General Synthetic Procedure B, Method D. After the usual work-up, the product was isolated as a light brown oil (130.0 mg, 85%).

HPLC 100%, Rt=2.18 min.

¹H-NMR (250 MHz, CDCl₃) δ=2.16-2.37 (m, 2H), 2.62-2.76 (m, 3H), 2.82-2.89 (m, 1H), 2.90-3.00 (m, 2H), 3.17-3.27 (m, 2H), 3.89-3.99 (m, 1H), 4.04-4.15 (m, 3H), 6.91-6.98 (m, 3H), 7.04-7.32 (m, 5H), 7.48 (d, 1H, J=7.3 Hz), 8.88 (brs, 1H).

MS (AP) m/z 349 (M+H)⁺.

### Example 27

### N-Ethyl-6-methoxy-1′-(2-phenoxyethyl)-4,9-dihydrospiro[beta-carboline-1,3′-pyrrolidine]-2(3H)-carboxamide

EXAMPLE 10 (50 mg, 0.13 mmol) was dissolved in DCM (1 mL), ethyl isocyanate (13 μL, 0.16 mmol) was added. The solution was left overnight and the excess isocyanate was removed by treatment with PS-Trisamine (50 mg, Argonaut). Flash-chromatography of the solution on 3 mL silica gel with CHCl₃ and 2% MeOH, NH₃/CHCl₃ gave the title compound (59 mg, 100%).

HPLC 99%, R_{T}=2.10 min (System A, 10-97% MeCN over 3 min).

¹H NMR (270 MHz, CDCl₃) δ 1.15 (t, J=7.2 Hz, 3H), 2.22-2.38 (m, 1H), 2.55-2.89 (m, 3H), 2.90-3.17 (m, 5H), 3.20-3.36 (m, 2H), 3.44 (d, J=9.4 Hz, 1H), 3.50-3.65 (m, 1H), 3.84 (s, 3H), 3.89-4.02 (m, 1H), 4.06 (t, J=5.2 Hz, 2H), 6.58 (m, 1H), 6.78 (dd, J=2.5, 8.7 Hz, 1H), 6.86-7.01 (m, 4H), 7.16 (d, J=8.7 Hz, 1H), 7.23-7.35 (m, 2H), 8.95 (s, 1H).

MS (ESI+) m/z (M+H)⁺.

### Example 28

### Ethyl ({[6-methoxy-1′-(2-phenoxyethyl)-4,9-dihydrospiro[beta-carboline-1,3′-pyrrolidin]-2(3H)-yl]carbonyl}amino)acetate

The title compound was prepared in the same way as EXAMPLE 27. Yield 64 mg, 97%.

HPLC 99%, R_{T}=2.08 min (System A, 10-97% MeCN over 3 min).

¹H NMR (270 MHz, CDCl₃) δ 1.18 (t, J=7.2 Hz, 3H), 2.27-2.43 (m, 1H), 2.62-3.07 (m, 7H), 3.20-3.34 (m, 1H), 3.44-3.59 (m, 1H), 3.66 (d, J=10.1 Hz, 1H), 3.84 (s, 3H), 3.93-4.11 (m, 6H), 4.15-4.28 (m, 1H), 6.80 (dd, J=2.5, 8.9 Hz, 1H), 6.83-7.00 (m, 4H), 7.20 (d, J=8.7 Hz, 1H), 7.22-7.33 (m, 2H), 8.21 (m, 1H), 8.64 (s, 1H).

MS (ESI+) m/z (M+H)⁺.

### Example 29

### N-Allyl-6-methoxy-1′-(2-phenoxyethyl)-4,9-dihydrospiro[beta-carboline-1,3′-pyrrolidine]-2(3H)-carboxamide

The title compound was prepared in a similar way as EXAMPLE 27. Yield 60 mg, 100%.

HPLC 99%, R_{T}=2.14 min (System A, 10-97% MeCN over 3 min).

¹H NMR (270 MHz, CDCl₃) δ2.24-2.39 (m, 1H), 2.55-2.90 (m, 3H), 2.90-3.05 (m, 3H), 3.06-3.19 (m, 2H), 3.43-3.64 (m, 2H), 3.80-3.93 (m, 5H), 3.98-4.12 (m, 3H), 5.02-5.23 (m, 2H), 5.80-5.98 (m, 1H), 6.79 (dd, J=2.5, 8.9 Hz, 1H), 6.85-7.09 (m, 5H), 7.17 (d, J=8.7 Hz, 1H), 7.25-7.35 (m, 2H), 8.93 (s, 1H).

MS (ESI+) m/z 461 (M+H)⁺.

### Example 30

### N-Butyl-6-methoxy-1′-(2-phenoxyethyl)-4,9-dihydrospiro[beta-carboline-1,3′-pyrrolidine]-2(3H)-carboxamide

The title compound was prepared in the same way as EXAMPLE 27. Yield 57 mg, 92%.

HPLC 99%, R_{T}=2.35 min (System A, 10-97% MeCN over 3 min).

¹H NMR (270 MHz, CDCl₃) δ 0.89 (t, J=7.2 Hz, 3H), 1.23-1.41 (m, 2H), 1.42-1.58 (m, 2H), 2.21-2.37 (m, 1H), 2.53-2.85 (m, 3H), 2.90-3.17 (m, 5H), 3.17-3.31 (m, 2H), 3.44 (d, J=9.4 Hz, 1H), 3.51-3.65 (m, 1H), 3.84 (s, 3H), 3.88-4.02 (m, 1H), 4.07 (t, J=5.2 Hz, 2H), 6.60 (m, 1H), 6.78 (dd, J=2.2, 8.7 Hz, 1H), 6.86-7.01 (m, 4H), 7.16 (d, J=8.7 Hz, 1H), 7.25-7.34 (m, 2H), 8.91 (s, 1H).

MS (ESI+) m/z 477 (M+H)⁺.

### Example 31

### N′-{2-[6-Methoxy-1′-(2-phenoxyethyl)-4,9-dihydrospiro[beta-carboline-1,3′-pyrrolidin]-2(3H)-yl]ethyl}-N,N-dimethylurea trifluoroacetate

2-[6-Methoxy-1′-(2-phenoxyethyl)-4,9-dihydrospiro[beta-carboline-1,3′-pyrrolidin]-2(3H)-yl]ethanamine (EXAMPLE 181, 100 mg, 0.2 mmol), K₂CO₃ (66 mg, 0.5 mmol), dimethylcarbamic chloride (28 mg, 0.3 mmol) and acetonitrile (2 mL) were stirred at 75° C. for 16 h. The mixture was filtered and the solvent was removed. The product was purified by preparative HPLC using acetonitrile-water gradients containing 0.1% trifluoroacetic acid. Yield: 53.8 mg (55%).

HPLC 95%, R_{T}: 1.937 (10-97% MeCN over 3 min).

¹H NMR (270 MHz, Methanol-d₃) δ ppm 2.63-2.71 (m, 2H) 2.81 (s, 6H) 3.09-3.14 (m, 2H) 3.59-3.80 (m, 12H) 3.81 (s, 3H) 4.34 (t, J=4.82 Hz, 2H) 6.85 (dd, J=8.91, 2.47 Hz, 1H) 6.95-6.98 (m, 4H) 7.23-7.32 (m, 3H).

MS (ESI+) m/z 492 (M+H)⁺.

### Example 32

### 2-Methoxy-N-{2-[6-methoxy-1′-(2-phenoxyethyl)-4,9-dihydrospiro[beta-carboline-1,3′-pyrrolidin]-2(3H)-yl]ethyl}acetamide trifluoroacetate

The title compound was prepared as described for EXAMPLE 31. Yield: 32.5 mg (33%).

HPLC 97%, R_{T}: 1.879 (10-97% MeCN over 3 min).

¹H NMR (270 MHz, Methanol-d₃) δ ppm 2.70-3.19 (m, 6H) 3.37 (s, 3H) 3.56-3.71 (m, 10H) 3.80 (s, 3H) 3.86 (s, 2H) 4.37 (t, J=4.82 Hz, 2H) 6.80-6.84 (m, 1H) 6.96-7.00 (m, 4H) 7.26-7.33 (m, 3H).

MS (ESI+) m/z 493 (M+H)⁺.

### Example 33

### 3-Amino-4-({2-[6-methoxy-1′-(2-phenoxyethyl)-4,9-dihydrospiro[beta-carboline-1,3′-pyrrolidin]-2(3H)-yl]ethyl}amino)cyclobut-3-ene-1,2-dione trifluoroacetate

3-Amino-4-ethoxycyclobut-3-ene-1,2-dione (48.99 mg, 0.3 mmol), NaOH (13.89 mg, 0.3 mmol), 2-[6-methoxy-1′-(2-phenoxyethyl)-4,9-dihydrospiro[beta-carboline-1,3′-pyrrolidin]-2(3H)-yl]ethanamine (EXAMPLE 181, 146.0 mg, 0.3 mmol) and ethanol (3 mL) were mixed together and stirred at rt for 16 h. The product was filtered, solvent was removed and the product was purified by preparative HPLC using acetonitrile-water gradients containing 0.1% trifluoroacetic acid. Yield: 9.3 mg (6%).

HPLC 100%, R_{T}: 1.742 (10-97% MeCN over 3 min).

¹H NMR (270 MHz, Methanol-d₃) δ ppm 2.58-2.76 (m, 4H) 2.85-3.14 (m, 4H) 3.38-3.42 (m, 2H) 3.49-3.55 (m, 2H) 3.71 (dd, J=10.27, 4.33 Hz, 4H) 3.79 (s, 3H) 4.35-4.38 (m, 2H) 6.77-6.81 (m, 1H) 6.92-7.03 (m, 5H) 7.21-7.34 (m, 2H).

MS (ESI+) m/z 516 (M+H)⁺.

### Example 34

### Ethyl ({[7-fluoro-1′-(2-phenoxyethyl)-4,9-dihydrospiro[beta-carboline-1,3′-pyrrolidin]-2(3H)-yl]carbonyl}amino)acetate trifluoro acetate

EXAMPLE 11 (21.9 mg, 0.046 mmol) and ethylacetoisocyanate (8.9 mg, 0.069 mmol) were dissolved in MeCN (500 μL) and allowed to stir at rt for 1 h. The reaction mixture was then diluted with MeCN (1 mL) and purified by direct injection to a preparative HPLC/UV System, MeCN:H₂O (0.1% TFA) 26-49% giving 16.1 mg (58%) white powder.

HPLC 92%, R_{T}=2.10 min (System A. 10-97% MeCN over 3 min), 91%, R_{T}=1.92 min (System B. 10-97% MeCN over 3 min).

¹H NMR (400 MHz, MeOD) δ ppm 1.23 (t, J=7.2 Hz, 3H) 2.64-2.71 (m, 1H) 2.79-2.87 (m, 3H) 3.58-3.83 (m, 6H) 3.92 (d, J=4.1 Hz, 2H) 4.10 (q, J=7.2 Hz, 2H) 4.15-4.17 (m, 1H) 4.20 (d, J=12.7 Hz, 1H) 4.28-4.38 (m, 2H) 6.82-6.87 (m, 1H) 6.98-7.08 (m, 3H) 7.08 (dd, J=9.8, 2.2 Hz, 1H) 7.28-7.32 (m, 2H) 7.43 (dd, J=8.6, 5.3 Hz, 1H).

MS (ESI+) for C₂₇H₃₁N₄O₄F m/z 495 (M+H)⁺.

### Example 35

### Ethyl ({[6-methyl-1′-(2-phenoxyethyl)-4,9-dihydrospiro[beta-carboline-1,3′-pyrrolidin]-2(3H)-yl]carbonyl}amino)acetate trifluoroacetate

EXAMPLE 82 and ethylacetoisocyanate (10.7 mg, 0.084 mmol) were dissolved in MeCN (500 μL) and allowed to stir at rt for 1 h. The reaction mixture was then diluted with MeCN (1 mL) and purified by direct injection to a preparative HPLC/UV System, MeCN:H₂O (0.1% TFA) 27-50% giving 18.4 mg (54%) of a white powder.

HPLC 93%, R_{T}=2.14 min (System A. 10-97% MeCN over 3 min), 93%, R_{T}=1.95 min (System B. 10-97% MeCN over 3 min).

¹H NMR (400 MHz, MeOD) δ ppm 1.23 (t, J=7.1 Hz, 3H) 2.54 (s, 3H) 2.74-2.87 (m, 4H) 3.70-3.79 (m, 6H) 3.92 (d, J=4.6 Hz, 2H) 4.12 (q, J=7.1 Hz, 3H) 4.21 (d, J=12.7 Hz, 1H) 4.35-4.40 (m, 2H) 6.96-7.03 (m, 5H) 7.29-7.33 (m, 3H).

MS (ESI+) for C₂₈H₃₄N₄O₄ m/z 491 (M+H)⁺.

### Example 37

### 4-[6-Methoxy-1′-(2-phenoxyethyl)-4,9-dihydrospiro[beta-carboline-1,3′-pyrrolidin]-2(3H)-yl]butanenitrile

The title compound was prepared according to General Synthetic Procedure E, Method A using 50 μmol of EXAMPLE 10 at 80° C. over the weekend. 6.3 mg (28%) of a clear oil was produced.

HPLC 92%, R_{T}=2.77 (System A, MeCN 5-60% over 3 min). HPLC 92%, R_{T}=2.53 (System B, MeCN 5-60% over 3 min).

¹H NMR (400 MHz, CD₃OD) δ ppm 2.08 (ddd, J=13.9, 7.2, 7.0 Hz, 1H) 2.56 (t, J=7.0 Hz, 2H) 2.59-2.67 (m, 1H) 2.69-2.77 (m, 1H) 2.79-2.89 (m, 1H) 2.95-3.24 (m, 3H) 3.34 (s, 1H) 3.45-3.66 (m, 5H) 3.69-3.78 (m, 1H) 3.80 (s, 3H) 3.95-4.08 (m, 1H) 4.33 (t, J=5.0 Hz, 2H) 6.81 (dd, J=8.8, 2.5 Hz, 1H) 6.93-7.02 (m, 4H) 7.23-7.32 (m, 3H).

MS (ESI+) for C₂₇H₃₂N₄O₂ m/z 445 (M+H)⁺.

### Example 38

### 2-[6-Methoxy-1′-(2-phenoxyethyl)-4,9-dihydrospiro[beta-carboline-1,3′-pyrrolidin]-2(3H)-yl]acetamide

DMF (30 mL) was added to 6-methoxy-1′-(2-phenoxyethyl)-2,3,4,9-tetrahydrospiro[beta-carboline-1,3′-pyrrolidine] (EXAMPLE 10, 1.79 g, 4.7 mmol), 2-bromoacetamide (0.65 g, 4.7 mmol) and K₂CO₃ (0.66 g, (4.7 mmol). The reaction was heated at 100° C. for 1.5 h, let to rt, filtered and the solvent was removed. The product was purified by flash chromatography by using initially chloroform 100% as eluent followed by chloroform/methanol 95/5. Yield: 175 g (84%).

HPLC 93%, R_{T}: 1.834 (10-97% MeCN over 3 min).

¹H NMR (270 MHz, Methanol-d₃) δ ppm 2.10-2.33 (m, 1H) 2.64-2.75 (m, 1H) 2.85 (s, 3H) 2.87-3.03 (m, 8H) 3.79 (s, 3H) 4.14 (t, J=5.44 Hz, 2H) 6.69 (dd, J=8.66, 2.47 Hz, 1H) 6.88-6.96 (m, 4H) 7.11-7.14 (m, 1H) 7.23-7.30 (m, 2H) 7.89 (s, 1H) 7.96 (s, 2H).

MS (ESI+) m/z 435 (M+H)⁺.

### Example 39

### N-Ethyl-4-[6-methoxy-1′-(2-phenoxyethyl)-4,9-dihydrospiro[beta-carboline-1,3′-pyrrolidin]-2(3H)-yl]butanamide

EXAMPLE 42 (15.4 mg, 31 mmol) was dissolved in ethanol (0.5 mL) and the mixture was saturated with gaseous ethylamine. Heating at 80° C. overnight and purification with preparative HPLC produced 1.9 mg (13%) clear oil.

HPLC 98%, R_{T}=12.66 (System A, MeCN 5-60% over 3 min). HPLC 97%, R_{T}=2.40 (System B, MeCN 5-60% over 3 min).

¹H NMR (400 MHz, CD₃OD) δ ppm 1.06 (t, J=7.3 Hz, 3H) 1.25-1.34 (m, 2H) 2.00-2.10 (m, 2H) 2.30-2.49 (m, J=7.4 Hz, 3H) 2.54 (s, 2H) 2.57-2.67 (m, 1H) 2.98-3.25 (m, 7H) 3.67 (s, 3H) 3.81 (s, 3H) 4.24 (t, J=5.1 Hz, 2H) 6.82 (dd, J=8.8, 2.4 Hz, 1H) 6.91-6.99 (m, 4H) 7.19-7.34 (m, 3H).

MS (ESI+) for C₂₉H₃₈N₄O₃ m/z 491 (M+H)⁺.

### Example 40

### 1-Methoxy-3-[6-methoxy-1′-(2-phenoxyethyl)-4,9-dihydrospiro[beta-carboline-1,3′-pyrrolidin]-2(3H)-yl]propan-2-ol trifluoroacetate

6-Methoxy-1′-(2-phenoxyethyl)-2,3,4,9-tetrahydrospiro[beta-carboline-1,3′-pyrrolidine] (EXAMPLE 10, 66 mg, 0.2 mmol, K₂CO₃ (48 mg, 0.3 mmol), 1-chloro-3-methoxypropan-2-ol (33 mg, 0.3 mmol) and acetonitrile (2 mL) were stirred at 75° C. for 16 h. More K₂CO₃ (48 mg, 0.3 mmol), 1-chloro-3-methoxypropan-2-ol (33 mg, 0.3 mmol) were added to the reaction and 1-chloro-3-methoxypropan-2-ol (33 mg, 0.3 mmol) was added after 24 h. After another 24 h, the mixture was filtered and the solvent was removed. The product was purified by preparative HPLC using acetonitrile-water gradients containing 0.1% trifluoroacetic acid. Yield: 7.1 mg (8%).

HPLC 100%, R_{T}: 1.930 (10-97% MeCN over 3 min).

¹H NMR (270 MHz, Methanol-d₃) δ ppm 2.50-2.60 (m, 1H) 2.76-2.87 (m, 1H) 3.04 (t, J=6.06 Hz, 2H) 3.24-3.32 (m, 3H) 3.34-3.54 (m, 8H) 3.72-3.78 (m, 2H) 3.81 (s, 3H) 4.03-4.15 (m, 1H) 4.30 (t, J=4.95 Hz, 2H) 6.83 (dd, J=8.78, 2.35 Hz, 1H) 6.97-7.00 (m, 4H) 7.25-7.32 (m, 3H).

MS (ESI+) m/z 466 (M+H)⁺.

### Example 41

### Enantiomer (NB—The Chirality of the Compound is Relative)

### N-Ethyl-6-methoxy-1′-(2-phenoxyethyl)-4,9-dihydrospiro[beta-carboline-1,3′-pyrrolidine]-2(3H)-carboxamide

6-Methoxy-1′-(2-phenoxyethyl)-2,3,4,9-tetrahydrospiro[beta-carboline-1,3′-pyrrolidine] (EXAMPLE 51, 11.0 mg, 29 mmol) was mixed with ethyl isocyanate (6.0 uL, 98 umol), pyridine (6.3 uL, 78 umol) in dry DCM (2 mL) overnight at rt. The solvent was evaporated, the residue dissolved in MeOH with TFA (50 uL) and purified preparative HPLC (System A) to 6.6 mg (37%) clear oil.

HPLC 91%, R_{T}=1.71 (System A, MeCN 30-80% over 3 min). HPLC 92%, R_{T}=1.50 (System B, MeCN 30-80% over 3 min).

MS (ESI+) for C₂₆H₃₂N₄O₃ m/z 449 (M+H)⁺.

### Example 42

### Ethyl 4-[6-methoxy-1′-(2-phenoxyethyl)-4,9-dihydrospiro[beta-carboline-1,3′-pyrrolidin]-2(3H)-yl]butanoate

The title compound was prepared according to General Synthetic Procedure E, Method A using 125 mg of EXAMPLE 10 (0.33 mmol) at 80° C. for 2 h. 76.3 mg (47%) of a clear oil was produced.

HPLC 93%, R_{T}=2.93 (System A, MeCN 5-60% over 3 min). HPLC 93%, R_{T}=2.66 (System B, MeCN 5-60% over 3 min).

¹H NMR (400 MHz, CHLOROFORM-D) δ ppm 1.24 (t, J=7.2 Hz, 3H) 1.81-1.94 (m, 2H) 2.12-2.26 (m, 2H) 2.33-2.44 (m, 3H) 2.46-2.55 (m, 2H) 2.58 (d, J=8.3 Hz, 1H) 2.66-2.76 (m, 1H) 2.80-2.89 (m, 1H) 2.92-3.07 (m, 3H) 3.13-3.20 (m, 2H) 3.29 (td, J=8.7, 3.3 Hz, 1H) 3.84 (s, 3H) 4.07-4.17 (m, 4H) 6.76 (dd, J=8.7, 2.4 Hz, 1H) 6.92 (d, J=2.5 Hz, 1H) 6.95-7.02 (m, 3H) 7.12 (d, J=8.8 Hz, 1H) 7.28-7.38 (m, 2H) 9.28 (s, 1H).

MS (ESI+) for C₂₉H₃₇N₃O₄ m/z 492 (M+H)⁺.

### Example 43

### 6-Methoxy-2-(3-methoxypropyl)-1′-(2-phenoxyethyl)-2,3,4,9-tetrahydrospiro[beta-carboline-1,3′-pyrrolidine]

The title compound was prepared according to General Synthetic Procedure E, Method A using 50 μmol of EXAMPLE 10 at 80° C. over the weekend. 4.3 mg (19%) of a clear oil was produced.

HPLC 92%, R_{T}=2.70 (System A, MeCN 5-60% over 3 min). HPLC 91%, R_{T}=2.44 (System B, MeCN 5-60% over 3 min).

¹H NMR (400 MHz, CD₃OD) δ ppm 2.02-2.12 (m, 2H) 2.49-2.60 (m, 1H) 2.68-2.79 (m, 1H) 3.05 (t, J=6.0 Hz, 2H) 3.28 (s, 3H) 3.33-3.43 (m, 5H) 3.46 (t, J=5.6 Hz, 2H) 3.48-3.59 (m, 2H) 3.75 (t, J=5.9 Hz, 2H) 3.81 (s, 3H) 3.83-3.98 (m, 1H) 4.27 (t, J=5.1 Hz, 2H) 6.83 (dd, J=8.9, 2.4 Hz, 1H) 6.93-7.00 (m, 4H) 7.24-7.32 (m, 3H).

MS (ESI+) for C₂₇H₃₅N₃O₃ m/z 450 (M+H)⁺.

### Example 44

### 3-Ethoxy-4-({2-[6-methoxy-1′-(2-phenoxyethyl)-4,9-dihydrospiro[beta-carboline-1,3′-pyrrolidin]-2(3H)-yl]ethyl}amino)cyclobutane-1,2-dione trifluoroacetate

3,4-Diethoxycyclobut-3-ene-1,2-dione (0.024 mL, 0.2 mmol), NaOH (6.62 mg, 0.2 mmol), 2-[6-methoxy-1′-(2-phenoxyethyl)-4,9-dihydrospiro[beta-carboline-1,3′-pyrrolidin]-2(3H)-yl]ethanamine (EXAMPLE 181, 69.6 mg, 0.2 mmol) and ethanol (2 mL) were mixed together and stirred at rt for 1 h. The solvent was removed and the product was purified by preparative HPLC using acetonitrile-water gradients containing 0.1% trifluoroacetic acid. Yield: 8.8 mg (8%).

HPLC 100%, R_{T}: 1.994 (10-97% MeCN over 3 min).

¹H NMR (500 MHz, MeOD) δ ppm 1.38-1.43 (m, 3H) 2.65 (s, 2H) 2.86-3.09 (m, 2H) 3.33-3.39 (m, 8H) 3.64-3.77 (m, 6H) 3.80 (s, 3H) 4.34-4.37 (m, 2H) 6.79 (dd, J=8.79, 2.20 Hz, 1H) 6.93 (d, J=2.20 Hz, 1H) 6.99-7.00 (m, 3H) 7.21-7.23 (m, 1H) 7.28-7.32 (m, 2H).

MS (ESI+) m/z 545 (M+H)⁺.

### Example 45

### 6-Methoxy-2-methyl-1′-(2-phenoxyethyl)-2,3,4,9-tetrahydrospiro[beta-carboline-1,3′-pyrrolidine]

6-Methoxy-1′-(2-phenoxyethyl)-2,3,4,9-tetrahydrospiro[beta-carboline-1,3′-pyrrolidine] (EXAMPLE 10, 25 mg, 66 mmol) was dissolved in 1,2-dichloroethane (250 uL) and 37% water solution of formaldehyde (8.1 uL, 100 mmol). Triacetoxyborohydride (28 mg, 0.1 mmol) was added and the mixture was shaked at rt overnight. Evaporation, dissolution in MeOH, filtering and preparative HPLC (System B) to 5.8 mg (22%) tea-colored gum.

HPLC 99%, R_{T}=2.20 (System B, MeCN 5-60% over 3 min). HPLC 98%, R_{T}=3.53 (System C, MeCN 5-60% over 3 min).

¹H NMR (400 MHz, CD₃OD) δ ppm 2.21-2.32 (m, 1H) 2.39-2.49 (m, 1H) 2.70 (s, 3H) 2.76-3.24 (m, 8H) 3.32-3.41 (m, 2H) 3.79 (s, 3H) 4.20 (t, J=5.3 Hz, 2H) 6.75 (dd, J=8.8, 2.5 Hz, 1H) 6.89-7.00 (m, 4H) 7.18 (d, J=8.8 Hz, 1H) 7.24-7.30 (m, 2H).

MS (ESI+) for C₂₄H₂₉N₃O₂ m/z 392 (M+H)⁺.

### Example 46

### 6-Methoxy-2-[2-(2-methoxyethoxy)ethyl]-1′-(2-phenoxyethyl)-2,3,4,9-tetrahydrospiro[beta-carboline-1,3′-pyrrolidine]

The title compound was prepared according to General Synthetic Procedure E, Method A using 50 μmol of EXAMPLE 10 at 80° C. over the weekend. 3.7 mg (15%) of a clear oil was produced.

HPLC 97%, R_{T}=2.67 (System A, MeCN 5-60% over 3 min). HPLC 96%, R_{T}=2.42 (System B, MeCN 5-60% over 3 min).

¹H NMR (400 MHz, CD₃OD) δ ppm 2.43-2.55 (m, 1H) 2.70-2.79 (m, 1H) 3.05 (t, J=6.0 Hz, 2H) 3.26 (s, 2H) 3.32-3.41 (m, 5H) 3.43-3.58 (m, 4H) 3.58-3.68 (m, 3H) 3.68-3.79 (m, 3H) 3.81 (s, 3H) 3.83-3.93 (m, 2H) 4.26 (t, J=5.0 Hz, 2H) 6.83 (dd, J=8.9, 2.4 Hz, 1H) 6.93-7.00 (m, 4H) 7.24-7.33 (m, 3H).

MS (ESI+) for C₂₈H₃₇N₃O₄ m/z 480 (M+H)⁺.

### Example 47

### 2-[6-Methoxy-1′-(2-phenoxyethyl)-4,9-dihydrospiro[beta-carboline-1,3′-pyrrolidin]-2(3H)-yl]-2-oxoethanol trifluoroacetate

The title compound was prepared according to the General Synthetic Procedure F affording 10.5 mg (29%).

HPLC 100%, R_{T}=1.79 min (System A. 10-97% MeCN), 100%, R_{T}=2.36 min (System B. 5-60% MeCN).

¹H NMR (400 MHz, MeOD) δ ppm 2.62-2.97 (m, 4H) 3.06-3.16 (m, 0.4H) 3.43-3.55 (m, 0.6H) 3.61-3.93 (m, 6H) 4.08-4.70 (m, 8H) 6.78-6.88 (m, 1H) 6.93-7.08 (m, 4H) 7.22-7.39 (m, 3H).

MS (ESI+) m/z 436 (M+H)⁺.

HRMS (EI) calcd for C₂₅H₂₉N₃O₄: 435.2158, found 435.2145.

### Example 48

### Ethyl 5-[6-methoxy-1′-(2-phenoxyethyl)-4,9-dihydrospiro[beta-carboline-1,3′-pyrrolidin]-2(3H)-yl]pentanoate

The title compound was prepared according to General Synthetic Procedure E, Method A using 125 mg of EXAMPLE 10 (0.33 mmol) at 80° C. for 2 h. 64.7 mg (39%) of a clear oil was produced.

HPLC 94%, R_{T}=2.99 (System A, MeCN 5-60% over 3 min). HPLC 95%, R_{T}=2.72 (System B, MeCN 5-60% over 3 min).

¹H NMR (400 MHz, CHLOROFORM-D) δ ppm 1.25 (t, J=7.2 Hz, 3H) 1.54-1.74 (m, 4H) 2.09-2.27 (m, 2H) 2.30-2.40 (m, 3H) 2.45-2.55 (m, 2H) 2.59 (d, J=8.3 Hz, 1H) 2.62-2.72 (m, 1H) 2.77-2.88 (m, 1H) 2.92-3.08 (m, 3H) 3.15-3.23 (m, 2H) 3.28 (td, J=8.8, 3.3 Hz, 1H) 3.84 (s, 3H) 4.09-4.17 (m, 4H) 6.75 (dd, J=8.8, 2.5 Hz, 1H) 6.91 (d, J=2.5 Hz, 1H) 6.94-7.01 (m, 3H) 7.11 (d, J=8.8 Hz, 1H) 7.28-7.38 (m, 2H) 9.28 (s, 1H).

MS (ESI+) for C₃₀H₃₉N₃O₄ m/z 506 (M+H)⁺.

### Example 49

### 4-[6-Methoxy-1′-(2-phenoxyethyl)-4,9-dihydrospiro[beta-carboline-1,3′-pyrrolidin]-2(3H)-yl]butanoic acid

EXAMPLE 42 (15.4 mg, 31 mmol) was dissolved in ethanol (0.5 mL) and treated with 1M NaOH (xs, 500 uL). Heating at 80° C. overnight and purification with preparative HPLC produced 5.0 mg (35%) clear oil.

HPLC 98%, R_{T}=2.49 (System A, MeCN 5-60% over 3 min). HPLC 97%, R_{T}=2.27 (System C, MeCN 5-60% over 3 min).

¹H NMR (400 MHz, CD₃OD) δ ppm 1.89-2.07 (m, 2H) 2.24-2.34 (m, 1H) 2.38-2.59 (m, 3H) 2.90-3.28 (m, 8H) 3.43 (d, J=11.2 Hz, 1H) 3.46-3.68 (m, 3H) 3.79 (s, 3H) 4.19 (t, J=5.3 Hz, 2H) 6.78 (dd, J=8.8, 2.4 Hz, 1H) 6.89-6.99 (m, 4H) 7.21 (d, J=8.8 Hz, 1H) 7.23-7.30 (m, 2H).

MS (ESI+) for C₂₇H₃₃N₃O₄ m/z 464 (M+H)⁺.

### Example 50

### 2-Allyl-6-methoxy-1′-(2-phenoxyethyl)-2,3,4,9-tetrahydrospiro[beta-carboline-1,3′-pyrrolidine]

The title compound was prepared according to General Synthetic Procedure E, Method A using 33 mmol EXAMPLE 10 at rt overnight. 1.6 mg (11%) of a clear oil was produced.

HPLC 94%, R_{T}=2.80 (System A, MeCN 5-60% over 3 min). HPLC 94%, R_{T}=2.55 (System B, MeCN 5-60% over 3 min). ¹H NMR (400 MHz, CD₃OD) δ ppm 2.51 (dd, J=8.5, 5.3 Hz, 1H) 2.65-2.74 (m, 1H) 2.93-3.03 (m, 2H) 3.16-3.28 (m, 2H) 3.34 (s, 2H) 3.38-3.50 (m, 1H) 3.62 (t, J=5.6 Hz, 2H) 3.70-3.78 (m, 1H) 3.80 (s, 3H) 3.83-4.03 (m, 2H) 4.26 (t, J=5.1 Hz, 2H) 5.51 (d, J=10.3 Hz, 1H) 5.56 (d, J=16.8 Hz, 1H) 5.95-6.08 (m, 1H) 6.82 (dd, J=8.9, 2.4 Hz, 1H) 6.92-6.99 (m, 4H) 7.22-7.32 (m, 3H).

MS (ESI+) for C₂₆H₃₁N₃O₂ m/z 418 (M+H)⁺.

### Example 51

### Enantiomer (NB—The Chirality of the Compound is Relative)

### 6-Methoxy-1′-(2-phenoxyethyl)-2,3,4,9-tetrahydrospiro[beta-carboline-1,3′-pyrrolidine]

6-Methoxy-2,3,4,9-tetrahydrospiro[beta-carboline-1,3′-pyrrolidine] (COMPARATIVE EXAMPLE 182, 0.59 g, 2.3 mmol was suspended in DMSO (2.2 mL) and DIPEA (1.37 g, 10.6 mmol) was added. β-Bromophenetole (0.49 g, 2.4 mmol) in DMSO (4.4 mL) was added and the solution was stirred at rt overnight. An additional amount of β-bromophenetole (0.22 g, 1.1 mmol) was added and the reaction mixture was heated to 60° C. for 4 h. When no progress of the reaction was achieved the reaction was aborted to avoid more dialkylation. The reaction mixture was diluted with water and extracted one time with EtOAc. The organic phase was dried over Na₂SO₄, filtered and the solvent was removed at reduced pressure and the remaining brown oil was chromatographed on a column of silica (μ=45 mm, L=110) mm with initially CHCl₃ 100% as eluent followed by CHCl₃/MeOH/aq conc NH₃ 95/5/0.2 to give 2.18 g (5.78 mmol, 72.2%) of a light brown oil.

The brown oil was precipitated as its hydrochloric acid salt from EtOAc/ether 20/80 with HCl/ether to give a white powder, 330 mg (31%).

HPLC 93%, R_{T}=1.56 min (System A. 10-97% MeCN), 93%, R_{T}=1.39 min (System B. 10-97% MeCN).

¹H NMR (400 MHz, DEUTERIUM OXIDE) δ ppm 2.52-2.60 (m, 1H) 2.67-2.75 (m, 1H) 3.04 (t, J=6.0 Hz, 2H) 3.35-3.45 (m, 3H) 3.48-3.58 (m, 3H) 3.62-3.66 (m, 2H) 3.91 (s, 3H) 4.36 (t, J=4.9 Hz, 2H) 7.00 (dd, J=8.9, 2.6 Hz, 1H) 7.08-7.14 (m, 3H) 7.20 (d, J=2.3 Hz, 1H) 7.41-7.46 (m, 3H).

MS (ESI+) m/z 378 (M+H)⁺.

alt.

COMPARATIVE EXAMPLE 182 and K₂CO₃ were weighed into a 16 mm tube. A solution of β-bromophenetole (16.4 mg in 1 mL CH₃CN, 1.05 eq) was added to the reaction mixture. The tube was heated at 70° C. for 12 h. The reaction mixture was evaporated and dissolved in MeOH (1 mL) with TFA (50 uL). Filtration and purification with preparative HPLC isolated 22 mg (57%) of a yellow oil.

HPLC 100%, R_{T}=2.24 (System A, MeCN 5-60% over 3 min). HPLC 100%, R_{T}=2.04 (System B, MeCN 5-60% over 3 min).

¹H NMR identical to EXAMPLE 81.

MS (ESI+) for C₂₃H₂₇N₃O₂ m/z 378 (M+H)⁺.

[α]_{D} −31.6° (c=0.43 MeOH).

### Example 52

### N-Ethyl-5-[6-methoxy-1′-(2-phenoxyethyl)-4,9-dihydrospiro[beta-carboline-1,3′-pyrrolidin]-2(3H)-yl]pentanamide

EXAMPLE 48 (15.8 mg, 31 mmol) was dissolved in ethanol (0.5 mL) and the mixture was saturated with gaseous ethylamine. Heating at 80° C. overnight and purification with preparative HPLC produced 10.2 mg (65%) of a clear oil.

HPLC 95%, R_{T}=2.57 (System A, MeCN 5-60% over 3 min). HPLC 96%, R_{T}=2.31 (System B, MeCN 5-60% over 3 min).

¹H NMR (400 MHz, CD₃OD) δ ppm 1.07 (t, J=7.3 Hz, 3H) 1.67 (q, J=7.2 Hz, 2H) 1.82-1.93 (m, 2H) 2.22 (t, J=7.1 Hz, 2H) 2.54 (s, 2H) 2.70-2.83 (m, 1H) 2.88-3.01 (m, 1H) 3.07-3.12 (m, 2H) 3.15 (q, J=7.3 Hz, 2H) 3.57-3.74 (m, J=15.8, 4.4 Hz, 3H) 3.75-3.95 (m, 4H) 3.81 (s, 3H) 4.36 (t, J=4.8 Hz, 3H) 6.87 (dd, J=8.8, 2.4 Hz, 1H) 6.95-7.01 (m, 4H) 7.29 (t, J=8.2 Hz, 3H).

MS (ESI+) for C₃₀H₄₀N₄O₃ m/z 505 (M+H)⁺.

### Example 53

### N-Benzyl-2-[6-methoxy-1′-(2-phenoxyethyl)-4,9-dihydrospiro[beta-carboline-1,3′-pyrrolidin]-2(3H)-yl]-2-oxoethanamine trifluoroacetate

The title compound was prepared according to the General Synthetic Procedure F affording 2.2 mg (6%).

HPLC 100%, R_{T}=1.67 min (System A. 10-97% MeCN), 99%, R_{T}=1.38 min (System B. 10-90% MeCN).

¹H NMR (400 MHz, MeOD) δ ppm 2.71-2.84 (m, 2H) 2.85-2.94 (m, 2H) 3.51-3.65 (m, 1H) 3.69-3.85 (m, 5H) 3.85-3.99 (m, 3H) 4.20-4.32 (m, 3H) 4.32-4.48 (m, 5H) 6.85 (dd, J=8.78, 2.38 Hz, 1H) 6.93-7.06 (m, 4H) 7.19-7.35 (m, 3H) 7.41-7.56 (m, 5H).

MS (ESI+) m/z 525 (M+H)⁺.

HRMS (EI) calcd for C₃₂H₃₆N₄O₃: 524.2787, found 524.2781.

### Example 54

### Methyl ({2-[6-methoxy-1′-(2-phenoxyethyl)-4,9-dihydrospiro[beta-carboline-1,3′-pyrrolidin]-2(3H)-yl]-2-oxoethyl}amino)acetate trifluoroacetate

The title compound was prepared according to the General Synthetic Procedure F affording 2.6 mg (8%).

HPLC 98%, R_{T}=1.49 min (System A. 10-97% MeCN), 98%, R_{T}=1.18 min (System B. 10-90% MeCN).

¹H NMR (400 MHz, MeOD) δ ppm 2.71-2.93 (m, 4H) 3.52-3.62 (m, 1H) 3.71-3.77 (m, 1H) 3.78-3.94 (m, 10H) 4.05 (s, 2H) 4.20-4.29 (m, 1H) 4.35-4.51 (m, 5H) 6.85 (dd, J=8.72, 2.45 Hz, 1H) 6.95-7.07 (m, 4H) 7.22-7.38 (m, 3H).

MS (ESI+) m/z 507 (M+H)⁺.

HRMS (EI) calcd for C₂₈H₃N₄O₅: 506.2529, found 506.2519.

### Example 55

### Ethyl [6-methoxy-1′-(2-phenoxyethyl)-4,9-dihydrospiro[beta-carboline-1,3′-pyrrolidin]-2(3H)-yl]acetate

The title compound was prepared according to General Synthetic Procedure E, Method A using 125 mg of EXAMPLE 10 (0.33 mmol) at 80° C. for 2 h. 32.8 mg (21%) of a clear oil was produced.

HPLC 94%, R_{T}=3.12 (System A, MeCN 5-60% over 3 min). HPLC 94%, R_{T}=2.88 (System B, MeCN 5-60% over 3 min).

¹H NMR (400 MHz, CHLOROFORM-D) δ ppm 1.26 (t, J=7.0 Hz, 3H) 2.15-2.25 (m, 1H) 2.28-2.38 (m, 1H) 2.50-2.61 (m, 2H) 2.66 (d, J=8.3 Hz, 1H) 2.83-3.06 (m, 3H) 3.10-3.20 (m, 2H) 3.23-3.32 (m, 3H) 3.48 (d, J=16.3 Hz, 1H) 3.84 (s, 3H) 4.14 (t, J=5.1 Hz, 2H) 4.20 (q, J=7.3 Hz, 2H) 6.76 (dd, J=8.8, 2.5 Hz, 1H) 6.91 (d, J=2.3 Hz, 1H) 6.94-7.01 (m, 3H) 7.11 (d, J=8.8 Hz, 1H) 7.28-7.37 (m, 2H) 9.24 (s, 1H).

MS (ESI+) for C₂₇H₃₃N₃O₄ m/z 464 (M+H)⁺.

### Example 56

### N-(3,4-Dimethoxybenzyl)-2-[6-methoxy-1′-(2-phenoxyethyl)-4,9-dihydrospiro[beta-carboline-1,3′-pyrrolidin]-2(3H)-yl]-2-oxoethanamine trifluoroacetate

The title compound was prepared according to the General Synthetic Procedure F affording 7.8 mg (20%).

HPLC 99%, R_{T}=1.64 min (System A. 10-97% MeCN), 95%, R_{T}=1.36 min (System B. 10-90% MeCN).

MS (ESI+) m/z 585 (M+H)⁺.

HRMS (EI) calcd for C₃₄H₄₀N₄O₅: 584.2999, found 584.2974.

### Example 57

### 5-[6-Methoxy-1′-(2-phenoxyethyl)-4,9-dihydrospiro[beta-carboline-1,3′-pyrrolidin]-2(3H)-yl]-5-oxopentan-2-one trifluoroacetate

6-Methoxy-1′-(2-phenoxyethyl)-2,3,4,9-tetrahydrospiro[beta-carboline-1,3′-pyrrolidine] (EXAMPLE 10, 60 mg, 0.16 mmol), N,N′-diisopropylcarbodiimide (32 μL, 0.21 mmol), 4-oxopentanoic acid (24 mg, 0.21 mmol) and DCM (1 mL) were shaken at ambivalent temperature for 16 h. More N,N′-diisopropylcarbodiimide (32 μL, 0.21 mmol) and 4-oxopentanoic acid (24 mg, 0.21 mmol) were added to the reaction, which was shaken an additional 24 h and than the solvent was removed. The product was purified by preparative HPLC using acetonitrile-water gradients containing 0.1% triflouroacetic acid. Yield: 7.3 mg (10%).

HPLC 100%, R_{T}: 2.045 (10-97% MeCN over 3 min).

¹H NMR (270 MHz, Methanol-d₃) δ ppm 2.09 (s, 3H) 2.68-2.88 (m, 8H) 3.55-3.75 (m, 4H) 3.81 (s, 3H) 4.11-4.25 (m, 4H) 4.31-4.36 (m, 2H) 6.83 (dd, J=8.78, 2.35 Hz, 1H) 6.92-7.10 (m, 4H) 7.19-7.39 (m, 3H).

MS (ESI+) m/z 476 (M+H)⁺.

### Example 58

### Enantiomer (NB—The Chirality of the Compound is Relative)

### 6-Methoxy-1′,2-bis(2-phenoxyethyl)-2,3,4,9-tetrahydrospiro[beta-carboline-1,3′-pyrrolidine]

2.0 mg (4%) of a yellow oil was obtained as a side-product in the synthesis of EXAMPLE 51.

HPLC 98%, R_{T}=2.11 (System A, MeCN 30-80% over 3 min). HPLC 98%, R_{T}=3.01 (System C, MeCN 30-80% over 3 min).

¹H NMR identical to EXAMPLE 51.

MS (ESI+) for C₃₁H₃₅N₃O₃ m/z 498 (M+H)⁺.

### Example 60

### N-{2-[6-Methoxy-1′-(2-phenoxyethyl)-4,9-dihydrospiro[beta-carboline-1,3′-pyrrolidin]-2(3H)-yl]ethyl}methanesulfonamide trifluoroacetate

2-[6-Methoxy-1′-(2-phenoxyethyl)-4,9-dihydrospiro[beta-carboline-1,3′-pyrrolidin]-2(3H)-yl]ethanamine (EXAMPLE 181, 100 mg, 0.2 mmol, K₂CO₃ (66 mg, 0.5 mmol), methanesulfonyl chloride (30 mg, 0.3 mmol) and acetonitrile (2 mL) were stirred at 75° C. for 16 h. The mixture was filtered and the solvent was removed. The product was purified by preparative HPLC using acetonitrile-water gradients containing 0.1% trifluoroacetic acid. Yield: 11.3 mg (11%).

HPLC 97%, R_{T}: 1.826 (10-97% MeCN over 3 min).

¹H NMR (270 MHz, Methanol-d₃) δ ppm 2.61-2.78 (m, 4H) 2.98 (s, 3H) 3.01-3.13 (m, 2H) 3.34-3.40 (m, 2H) 3.49-3.53 (m, 4H) 3.64-3.72 (m, 4H) 3.80 (s, 3H) 4.33-4.37 (m, 2H) 6.80 (dd, J=8.66, 2.47 Hz, 1H) 6.94-7.02 (m, 4H) 7.22-7.34 (m, 3H).

MS (ESI+) m/z 499 (M+H)⁺.

### Example 61

### 1-[6-Methoxy-1′-(2-phenoxyethyl)-4,9-dihydrospiro[beta-carboline-1,3′-pyrrolidin]-2(3H)-yl]propan-2-ol trifluoroacetate

6-Methoxy-1′-(2-phenoxyethyl)-2,3,4,9-tetrahydrospiro[beta-carboline-1,3′-pyrrolidine] (EXAMPLE 10, 66 mg, 0.2 mmol, K₂CO₃ (48 mg, 0.3 mmol), 1-chloropropan-2-ol (25 mg, 0.3 mmol) and acetonitrile (2 mL) were stirred at 75° C. for 16 h. More K₂CO₃ (48 mg, 0.3 mmol), 1-chloropropan-2-ol (25 mg, 0.3 mmol) were added to the reaction and 1-chloropropan-2-ol (25 mg, 0.3 mmol) was added again after 24 h and 48 h. After another 72 h, the mixture was filtered and the solvent was removed. The product was purified by preparative HPLC using acetonitrile-water gradients containing 0.1% trifluoroacetic acid. Yield: 6.98 mg (8%).

HPLC 100%, R_{T}: 1.893 (10-97% MeCN over 3 min).

¹H NMR (270 MHz, Methanol-d₃) δ ppm 1.18-1.20 (m, 3H), 2.45-2.69 (m, 1H) 2.77-2.88 (m, 1H) 3.06-3.09 (m, 2H) 3.42-3.60 (m, 4H) 3.73-3.88 (m, 2H) 3.81 (s, 3H) 4.06-4.20 (m, 1H) 4.30 (t, J=5.07 Hz, 2H) 6.83 (dd, J=8.78, 2.35 Hz, 1H) 6.94-7.00 (m, 4H) 7.25-7.30 (m, 3H).

MS (ESI+) m/z 436 (M+H)⁺.

### Example 62

### Ethyl 3-[6-methoxy-1′-(2-phenoxyethyl)-4,9-dihydrospiro[beta-carboline-1,3′-pyrrolidin]-2(3H)-yl]-3-oxopropanoate trifluoroacetate

6-Methoxy-1′-(2-phenoxyethyl)-2,3,4,9-tetrahydrospiro[beta-carboline-1,3′-pyrrolidine] (EXAMPLE 10, 50 mg, 0.1 mmol), triethylamine (18 μL, 0.1 mmol), methyl 3-chloro-3-oxopropanoate (18 mg, 0.1 mmol) and acetonitrile (1 mL) were shaken at ambivalent temperature for 1 h and the product was purified by preparative HPLC using acetonitrile-water gradients containing 0.1% triflouroacetic acid. Yield: 4.65 mg (9%).

HPLC 100%, R_{T}: 2.188 (10-97% MeCN over 3 min).

¹H NMR (270 MHz, Methanol-d₃) δ ppm 1.21-1.30 (m, 3H) 2.72-2.87 (m, 4H) 3.50-3.76 (m, 4H) 3.81 (s, 3H) 3.85-4.09 (m, 2H) 4.08-4.48 (m, 6H) 6.83 (dd, J=8.66, 2.47 Hz, 1H) 6.96-7.03 (m, 4H) 7.27-7.33 (m, 3H).

MS (ESI+) m/z 492 (M+H)⁺.

### Example 63

### 2-[6-Methoxy-1′-(2-phenoxyethyl)-4,9-dihydrospiro[beta-carboline-1,3′-pyrrolidin]-2(3H)-yl]ethanol

The title compound was prepared according to General Synthetic Procedure E, Method B using 33 μmol EXAMPLE 10 at rt overnight. 3.2 mg (23%) of a clear oil was produced.

HPLC 96%, R_{T}=2.40 (System A, MeCN 5-60% over 3 min). HPLC 96%, R_{T}=2.16 (System B, MeCN 5-60% over 3 min).

¹H NMR (400 MHz, CD₃OD) δ ppm 2.50-2.61 (m, 2H) 2.77 (s, J=14.7 Hz, 1H) 3.00-3.07 (m, 2H) 3.33-3.50 (m, 5H) 3.54-3.64 (m, 1H) 3.74-3.79 (m, 2H) 3.80 (s, 3H) 3.85-3.99 (m, 3H) 4.28 (t, J=5.1 Hz, 2H) 6.82 (dd, J=8.8, 2.4 Hz, 1H) 6.93-7.01 (m, 4H) 7.23-7.32 (m, 3H).

MS (ESI+) for C₂₅H₃₁N₃O₃ m/z 422 (M+H)⁺.

### Example 64

### 1-[6-Methoxy-1′-(2-phenoxyethyl)-4,9-dihydrospiro[beta-carboline-1,3′-pyrrolidin]-2(3H)-yl]-2-methylpropan-2-ol trifluoroacetate

6-Methoxy-1′-(2-phenoxyethyl)-2,3,4,9-tetrahydrospiro[beta-carboline-1,3′-pyrrolidine] (EXAMPLE 10, 66 mg, 0.2 mmol, K₂CO₃ (48 mg, 0.3 mmol), 1-chloro-2-methylpropan-2-ol (28 mg, 0.3 mmol) and acetonitrile (2 mL) were stirred at 75° C. for 16 h. More K₂CO₃ (48 mg, 0.3 mmol), 1-chloro-2-methylpropan-2-ol (28 mg, 0.3 mmol) were added to the reaction and 1-chloro-2-methylpropan-2-ol (28 mg, 0.3 mmol) was added again after 24 h and 48 h. After another 72 h, the mixture was filtered and the solvent was removed. The product was purified by preparative HPLC using acetonitrile-water gradients containing 0.1% trifluoroacetic acid. Yield: 6.96 mg (8%).

HPLC 98%, R_{T}: 2.099 (10-97% MeCN over 3 min).

¹H NMR (270 MHz, Methanol-d₃) δ ppm 1.26 (s, 3H) 1.35 (s, 3H) 2.46-2.53 (m, 1H) 2.84-3.04 (m, 1H) 3.04-3.14 (m, 2H) 3.47-3.66 (m, 6H) 3.81 (s, 3H) 3.84 (dd, J=6.68, 3.22 Hz, 2H) 4.18-4.29 (m, 1H) 4.32-4.35 (m, 2H) 6.81 (dd, J=8.91, 2.47 Hz, 1H) 6.95-7.02 (m, 4H) 7.23-7.33 (m, 3H).

MS (ESI+) m/z 450 (M+H)⁺.

### Example 65

### Methyl 5-[6-methoxy-1′-(2-phenoxyethyl)-4,9-dihydrospiro[beta-carboline-1,3′-pyrrolidin]-2(3H)-yl]-5-oxopentanoate trifluoroacetate

6-Methoxy-1′-(2-phenoxyethyl)-2,3,4,9-tetrahydrospiro[beta-carboline-1,3′-pyrrolidine] (EXAMPLE 10, 50 mg, 0.1 mmol), triethylamine (18 μL, 0.1 mmol), ethyl 4-chloro-4-oxobutanoate (19 μL, 0.1 mmol) and acetonitrile (1 mL) were shaken at ambivalent temperature for 1 h and the product was purified by preparative HPLC using acetonitrile-water gradients containing 0.1% triflouroacetic acid. Yield: 8.36 mg (17%).

HPLC 90%, R_{T}: 2.203 (10-97% MeCN over 3 min).

¹H NMR (270 MHz, Methanol-d₃) δ ppm 1.31 (t, J=7.30 Hz, 2H) 1.76-2.11 (m, 6H) 2.30-2.85 (m, 8H) 3.65 (s, 3H) 3.81 (s, 3H) 3.06-4.39 (m, 4H) 6.82 (dd, J=8.78, 2.35 Hz, 1H) 6.96-7.05 (m, 4H) 7.28-7.33 (m, 3H).

MS (ESI+) m/z 506 (M+H)⁺.

### Example 67

### 6-Methoxy-2-(methoxyacetyl)-1′-(2-phenoxyethyl)-2,3,4,9-tetrahydrospiro[beta-carboline-1,3′-pyrrolidine]trifluoroacetate

6-Methoxy-1′-(2-phenoxyethyl)-2,3,4,9-tetrahydrospiro[beta-carboline-1,3′-pyrrolidine] (EXAMPLE 10, 50 mg, 0.1 mmol), triethylamine (18 μL, 0.1 mmol), methoxyacetyl chloride (12 μL, 0.1 mmol) and acetonitrile (1 mL) were shaken at ambivalent temperature for 1 h and the product was purified by preparative HPLC using acetonitrile-water gradients containing 0.1% triflouroacetic acid. Yield: 1.9 mg (4%).

HPLC 97%, R_{T}: 2.059 (10-97% MeCN over 3 min).

¹H NMR (270 MHz, Methanol-d₃) δ ppm 2.84 (dd, J=7.67, 3.96 Hz, 2H) 3.21 (q, J=7.18 Hz, 4H) 3.44 (s, 3H) 3.48-3.79 (m, 4H) 3.81 (s, 3H) 3.85-4.02 (m, 2H) 4.30-4.48 (m, 4H) 6.83 (dd, J=8.78, 2.35 Hz, 1H) 6.99-7.05 (m, 4H) 7.27-7.33 (m, 3H).

MS (ESI+) m/z 450 (M+H)⁺.

### Example 68

### 6-Fluoro-1′-(2-phenoxyethyl)-2,3,4,9-tetrahydrospiro[beta-carboline-1,3′-pyrrolidine] trifluoroacetate

2-(3-Chloropropyl)-1,3-dioxolane (350 μL, 2.7 mmol) was added to (4-fluorophenyl)hydrazine (335 mg, 2.7 mmol) in ethanol (25 mL) and water (5 mL) and the reaction was heated at 95° C. for 1 h and the solvent was than removed in vacuo. The crude was purified with preparative HPLC using acetonitrile-water gradients containing 0.1% trifluoroacetic acid.

1-(2-Phenoxyethyl)pyrrolidin-3-one (COMPARATIVE EXAMPLE 183, 85 mg, 0.41 mmol) in acetic acid (1 mL) was added to 2-(5-fluoro-1H-indol-3-yl)ethanamine (73.6 mg, 0.41 mmol) and the reaction was heated at 100° C. for 1 h, diluted with methanol (2 mL) and purified by preparative HPLC using acetonitrile-water gradients containing 0.1% trifluoroacetic acid to afford 22.8 mg (15%).

HPLC 99%, R_{T}: 1.760 (10-97% MeCN over 3 min).

¹H NMR (270 MHz, Methanol-d₃) δ ppm 2.50-2.66 (m, 2H) 3.00-3.07 (m, 2H) 3.39-3.86 (m, 8H) 4.27-4.31 (m, 2H) 6.92-6.99 (m, 5H) 7.18 (dd, J=9.40, 2.47 Hz, 1H) 7.25-7.36 (m, 2H).

MS (ESI+) m/z 366 (M+H)⁺.

### Example 69

### 1′-[2-(4-Fluorophenoxy)ethyl]-6-methoxy-2,3,4,9-tetrahydrospiro[beta-carboline-1,3′-pyrrolidine]

The title compound was prepared according to General Synthetic Procedure A, Method A followed by General Synthetic Procedure B, Method A.

HPLC 97%.

### Example 70

### Enantiomer (NB—The Chirality of the Compound is Relative)

### 4-{2-[6-Methoxy-2,3,4,9-tetrahydro-1′H-spiro [beta-carboline-1,3′-pyrrolidin]-1′-yl]ethoxy}benzonitrile trifluoroacetate

6-Methoxy-2,3,4,9-tetrahydrospiro[beta-carboline-1,3′-pyrrolidine] (COMPARATIVE EXAMPLE 182, 30.0 mg, 0.12 mmol) was suspended in DMSO (200 μL) and DIPEA (114 μL, 0.65 mmol) was added. 2-(4-Cyanophenoxy)ethyl methanesulfonate (29.5 mg, 0.12 mmol) in DMSO (400 μL) was added and the solution was stirred at 50° C. overnight. The reaction mixture was diluted with water and extracted one time with EtOAc. The organic phase was dried over Na₂SO₄, filtered and the solvent was removed at reduced pressure and the remaining oil was then diluted with MeCN (1 mL) and purified by direct injection to a preparative HPLC/UV System, MeCN:H₂O (0.1% TFA) 20-41% giving a yellow oil, 9 mg, 15%.

HPLC 99%, R_{T}=1.544 min (System A. 10-97% MeCN), 99%, R_{T}=1.400 min (System B. 10-97% MeCN).

¹H NMR (400 MHz, MeOD) δ ppm 2.58-2.70 (m, 1H) 2.71-2.77 (m, 1H) 3.06 (t, J=6.0 Hz, 2H) 3.42-3.59 (m, 4H) 3.62-3.65 (m, 2H) 3.69-3.72 (m, 1H) 3.81 (s, 3H) 3.92 (d, J=12.5 Hz, 1H) 4.41 (t, J=4.9 Hz, 2H) 6.83 (dd, J=8.8, 2.4 Hz, 1H) 6.98 (d, J=2.3 Hz, 1H) 7.11-7.14 (m, 2H) 7.26 (d, J=8.8 Hz, 1H) 7.65-7.69 (m, 2H).

MS (ESI+) m/z 403 (M+H)⁺.

### Example 72

### 2-[(Ethylthio)acetyl]-6-methoxy-1′-(2-phenoxyethyl)-2,3,4,9-tetrahydrospiro[beta-carboline-1,3′-pyrrolidine] trifluoroacetate

The title compound was prepared according to the General Synthetic Procedure F affording 12.5 mg (38%).

HPLC 100%, R_{T}=2.62 min (System A. 10-97% MeCN), 99%, R_{T}=1.81 min (System B. 10-90% MeCN).

¹H NMR (400 MHz, MeOD) δ ppm 1.19 (t, J=7.34 Hz, 3H) 2.59 (q, J=7.40 Hz, 2H) 2.66-2.95 (m, 4H) 3.53-3.64 (m, 3H) 3.65-3.74 (m, 2H) 3.75-3.92 (m, 5H) 4.04-4.13 (m, 1H) 4.17-4.29 (m, 2H) 4.29-4.43 (m, 2H) 6.82 (dd, J=8.78, 2.51 Hz, 1H) 6.93-7.09 (m, 4H) 7.22-7.36 (m, 3H).

MS (ESI+) m/z 480 (M+H)⁺.

HRMS (EI) calcd for C₂₇H₃₃N₃O₃S: 479.2243, found 479.2233.

### Example 73

### N-Isopropyl-N-{2-[6-methoxy-1′-(2-phenoxyethyl)-4,9-dihydrospiro[beta-carboline-1,3′-pyrrolidin]-2(3H)-yl]-2-oxoethyl}propan-2-amine trifluoroacetate

The title compound was prepared according to the General Synthetic Procedure F affording 0.7 mg (2%).

HPLC 96%, R_{T}=1.63 min (System A. 10-97% MeCN), 94%, R_{T}=1.35 min (System B. 10-90% MeCN).

¹H NMR (400 MHz, MeOD) δ ppm 1.17 (d, J=6.40 Hz, 3H) 1.24-1.40 (m, J=6.40 Hz, 6H) 1.44 (d, J=6.53 Hz, 3H) 2.71-2.85 (m, 3H) 2.88-3.00 (m, 2H) 3.69-3.79 (m, 3H) 3.81 (s, 3H) 3.84-4.04 (m, 4H) 4.22-4.48 (m, 6H) 6.83-6.90 (m, 1H) 6.95-7.09 (m, 4H) 7.25-7.38 (m, 3H).

MS (ESI+) m/z 519 (M+H)⁺.

HRMS (EI) calcd for C₃₁H₄₂₂N₄O₃: 518.3257, found 518.3252.

### Example 74

### 4-[6-Methoxy-1′-(2-phenoxyethyl)-4,9-dihydrospiro[beta-carboline-1,3′-pyrrolidin]-2(3H)-yl]butanamide

EXAMPLE 42 (15.4 mg, 31 mmol) was dissolved in ethanol (0.5 mL) and treated with sat aq ammonia (0.5 mL). Heating at 80° C. overnight and purification with preparative HPLC produced 1.5 mg (10%) of a clear oil.

HPLC 92%, R_{T}=2.40 (System A, MeCN 5-60% over 3 min). HPLC 93%, R_{T}=2.16 (System B, MeCN 5-60% over 3 min).

¹H NMR (400 MHz, CD₃OD) δ ppm 2.06 (s, 2H) 2.34-2.68 (m, 4H) 2.54 (s, 2H) 2.97-3.14 (m, 4H) 3.14-3.24 (m, 3H) 3.58-3.73 (m, 3H) 3.80 (s, 3H) 4.24 (t, J=5.1 Hz, 2H) 6.81 (dd, J=8.8, 2.4 Hz, 1H) 6.91-6.99 (m, 4H) 7.21-7.32 (m, 3H).

MS (ESI+) for C₂₇H₃₄N₄O₃ m/z 463 (M+H)⁺.

### Example 75

### 1′-[2-(4-Cyanophenoxy)ethyl]-N-(3,5-dimethylisoxazol-4-yl)-6-methoxy-4,9-dihydrospiro[beta-carboline-1,3′-pyrrolidine]-2(3H)-carboxamide trifluoroacetate

9.6 mg (36%) of a yellow oil was prepared according to the same procedure as described in EXAMPLE 81 followed by General Synthetic Procedure C.

HPLC 95%, R_{T}=2.76 (System A, MeCN 5-60% over 3 min). HPLC 92%, R_{T}=2.59 (System B, MeCN 5-60% over 3 min).

¹H NMR (400 MHz, CD₃OD) δ ppm 2.08 (s, 3H) 2.25 (s, 3H) 2.68-2.80 (m, 1H) 2.80-2.87 (m, 1H) 2.87-2.96 (m, 2H) 3.62-3.90 (m, 8H) 3.96-4.05 (m, 1H) 4.12-4.21 (m, 1H) 4.27 (d, J=12.8 Hz, 1H) 4.37-4.51 (m, 2H) 6.83 (dd, J=8.8, 2.3 Hz, 1H) 6.97 (d, J=2.3 Hz, 1H) 7.01 (d, J=9.0 Hz, 2H) 7.28 (d, J=8.8 Hz, 1H) 7.63 (d, J=8.8 Hz, 2H).

MS (ESI+) for C₃₀H₃₂N₆O₄ m/z 541 (M+H)⁺.

### Example 76

### N,N-Diethyl-2-[6-methoxy-1′-(2-phenoxyethyl)-4,9-dihydrospiro[beta-carboline-1,3′-pyrrolidin]-2(3H)-yl]acetamide

The title compound was prepared according to General Synthetic Procedure E, Method A using 50 μmol EXAMPLE 10 at 80° C. over the weekend, 6.5 mg (26%) clear oil was produced.

HPLC 94%, R_{T}=3.10 (System A, MeCN 5-60% over 3 min). HPLC 94%, R_{T}=2.84 (System B, MeCN 5-60% over 3 min).

¹H NMR (400 MHz, CD₃OD) δ ppm 1.09 (dt, J=8.8, 7.2 Hz, 6H) 2.51-2.70 (m, 2H) 2.83-2.99 (m, 2H) 3.19-3.28 (m, 2H) 3.34-3.60 (m, 6H) 3.61-3.70 (m, 2H) 3.80 (s, 3H) 3.83-3.99 (m, 3H) 4.00-4.10 (m, 1H) 4.36 (t, J=4.8 Hz, 2H) 6.81 (dd, J=8.8, 2.4 Hz, 1H) 6.96 (d, J=2.3 Hz, 1H) 6.97-7.03 (m, 3H) 7.24 (d, J=8.8 Hz, 1H) 7.27-7.33 (m, 2H).

MS (ESI+) for C₂₉H₃₈N₄O₃ m/z 491 (M+H)⁺.

### Example 77

### Ethyl 6-methoxy-1′-(2-phenoxyethyl)-4,9-dihydrospiro[beta-carboline-1,3′-pyrrolidine]-2(3H)-carboxylate trifluoroacetate

Ethyl chloroformate (16 μm 0.173 mmol) was added to a precooled mixture (0° C.) of EXAMPLE 10 (50.0 mg, 0.132 mmol) and Et₃N (24 μL, 0.173 mmol) in DCM (5 mL). The reaction mixture was stirred for 1 h before it was allowed to take rt. The mixture was extracted between DCM and sat. NaHCO₃ (aq) followed by brine. The organic layer was dried (Na₂SO₄), filtered and evaporated. The crude was purified by preparative HPLC (30-70%, 0.1% TFA). The pooled fractions were evaporated using Speedvac providing 30 mg (40%) of product as TFA-salt.

HPLC 100%, R_{T}=2.16 min (System A. 10-97% MeCN), 100%, R_{T}=1.85 min (System B. 10-90% MeCN).

¹H NMR (400 MHz, MeOD) δ ppm 1.29 (t, J=7.09 Hz, 3H) 2.45-2.61 (m, 1H) 2.61-2.84 (m, 3H) 3.32-3.52 (m, J=3.64 Hz, 1H) 3.69-3.84 (m, 5H) 3.86-4.41 (m, 9H) 6.61-7.03 (m, 5H) 7.13-7.34 (m, 3H) 10.60 (s, 1H).

MS (ESI+) m/z 450 (M+H)⁺.

### Example 78

### 2-(1H-Imidazol-1-ylacetyl)-6-methoxy-1′-(2-phenoxyethyl)-2,3,4,9-tetrahydrospiro[beta-carboline-1,3′-pyrrolidine] trifluoroacetate

The title compound was prepared according to the General Synthetic Procedure F affording 4.6 mg (14%).

HPLC 100%, R_{T}=1.46 min (System A. 10-97% MeCN), 100%, R_{T}=1.12 min (System B. 10-90% MeCN).

¹H NMR (400 MHz, MeOD) δ ppm 2.70-2.81 (m, 1H) 2.82-3.02 (m, 3H) 3.59-3.73 (m, 3H) 3.76-3.90 (m, 5H) 4.00-4.09 (m, 1H) 4.13-4.21 (m, 1H) 4.22-4.39 (m, 3H) 5.55 (s, 2H) 6.78-6.88 (m, 3H) 6.96-7.02 (m, 2H) 7.23-7.34 (m, 3H) 7.55-7.60 (m, 1H) 7.64-7.69 (m, 1H) 8.90 (t, J=1.38 Hz, 1H).

MS (ESI+) m/z 486 (M+H)⁺.

HRMS (EI) calcd for C₂₈H₃₁N₅O₃: 485.2427, found 485.2414

### Example 79

### N-[2-(6-Methoxy-2,3,4,9-tetrahydro-1′H-spiro [beta-carboline-1,3′-pyrrolidin]-1′-yl)ethyl]aniline trifluoroacetate

6-Methoxy-2,3,4,9-tetrahydrospiro[beta-carboline-1,3′-pyrrolidine] hydrochloride (COMPARATIVE EXAMPLE 1, 0.050 g, 0.194 mmol) was suspended in DMSO (0.15 mL) and DIPEA (4.6 eq, 0.156 mL) was added. 2-Anilinoethyl methanesulfonate (0.054 g, 0.253 mmol) dissolved in DMSO (0.30 mL) was added and a solution was formed. The reaction mixture was agitated at rt until no more product was formed according to LC-MS (1 week).

The reaction mixture was diluted with MeCN and purification was performed using preparative LC (System A, 10-40% MeCN over 5 min) affording 0.0144 g (20%) of dark yellow gum.

HPLC 99% R_{T}=1.56 min (System A. 10-97% MeCN over 3 min), 99% R_{T}=1.36 min (System B. 10-97% MeCN over 3 min).

¹H NMR (270 MHz, METHANOL-D3) δ ppm 2.55-2.82 (m, 2H) 3.06 (t, J=6.06 Hz, 2H) 3.23-3.27 (m, 2H) 3.32-3.41 (m, 1H) 3.44-3.58 (m, 3H) 3.66 (q, J=5.86 Hz, 3H) 3.81 (s, 3H) 3.87 (d, J=112.37 Hz, 1H) 6.84 (dd, J=8.85, 2.41 Hz, 1H) 6.90-7.00 (m, 4H) 7.22-7.31 (m, 3H).

MS (ESI+) for C₂₃H₂₈N₄₀ m/z 377 (M+H)⁺.

### Example 80

### 6,8-Dimethyl-1′-(2-phenoxyethyl)-2,3,4,9-tetrahydrospiro[beta-carboline-1,3′-pyrrolidine] trifluoroacetate

2-(3-Chloropropyl)-1,3-dioxolane (350 μL, 2.7 mmol) was added to (2,4-dimethylphenyl)hydrazine (362 mg, 2.7 mmol) in ethanol (25 mL) and water (5 mL) and the reaction was heated at 95° C. for 1 h and the solvent was than removed in vacuo. The crude was purified with preparative HPLC using acetonitrile-water gradients containing 0.1% trifluoroacetic acid.

1-(2-Phenoxyethyl)pyrrolidin-3-one (COMPARATIVE EXAMPLE 183) in acetic acid (1 mL) was added to 2-(5,7-dimethyl-1H-indol-3-yl)ethanamine (58.7 mg, 0.26 mmol) and the reaction was heated at 100° C. for 1 h, diluted with methanol (2 mL) and purified by preparative HPLC using acetonitrile-water gradients containing 0.1% trifluoroacetic acid to afford 12.8 mg (13%).

HPLC 100%, R_{T}: 1.947 (10-97% MeCN over 3 min).

¹H NMR (270 MHz, Methanol-d₃) δ ppm 2.37 (s, 3H) 2.47 (s, 3H) 2.67-2.78 (m, 1H) 2.84-2.98 (m, 1H) 3.03-3.17 (m, 2H) 3.62-3.69 (m, 4H) 3.71-4.13 (m, 4 H) 4.35-4.39 (m, 2H) 6.85 (s, 1H) 6.97 (t, J=8.04 Hz, 3H) 7.12 (s, 1H) 7.26-7.32 (m, 2H).

MS (ESI+) m/z 376 (M+H)⁺.

### Example 81

### Enantiomer (NB—The Chirality of the Compound is Relative)

### 6-Methoxy-1′-(2-phenoxyethyl)-2,3,4,9-tetrahydrospiro[beta-carboline-1,3′-pyrrolidine]

COMPARATIVE EXAMPLE 186 and K₂CO₃ were weighed into a 16 mm tube. A solution of β-bromophenetole (16.4 mg in 1 mL CH₃CN, 1.05 eq) was added to the reaction mixture. The tube were heated at 70° C. for 12 h. The reaction mixture was evaporated and dissolved in MeOH (1 mL) with TFA (50 uL). Filtration and purification with preparative HPLC isolated 30 mg (77%) of a yellow oil.

HPLC 100%, R_{T}=2.24 (System A, MeCN 5-60% over 3 min). HPLC 100%, R_{T}=2.05 (System B, MeCN 5-60% over 3 min).

¹H NMR (270 MHz, METHANOL-D3) δ ppm 2.54-2.83 (m, 2H) 3.05 (t, J=6.1 Hz, 3H) 3.43-3.68 (m, 6H) 3.81 (s, 3H) 3.97 (m, 1H) 4.28-4.36 (m, 2H) 6.85 (dd, J=8.8, 2.4 Hz, 1H) 6.91-7.03 (m, 4H) 7.18-7.40 (m, 3H).

MS (ESI+) for C₂₃H₂₇N₃O₂ m/z 378 (M+H)⁺.

[α]_{D} +29.0° (c=0.41 MeOH).

### Example 82

### 6-Methyl-1′-(2-phenoxyethyl)-2,3,4,9-tetrahydrospiro[beta-carboline-1,3′-pyrrolidine]

The title compound was prepared according to General Synthetic Procedure A, Method C. Preparative HPLC/UV System, MeCN:H₂O (0.1% TFA) 18-40% afforded 37.8 mg (86%) white powder.

HPLC 100%, R_{T}=1.71 min (System A. 10-97% MeCN over 3 min), 100%, R_{T}=1.51 min (System B. 10-97% MeCN over 3 min).

¹H NMR (400 MHz, MeOD) δ ppm 2.4 (s, 1H) 2.51-2.67 (m, 2H) 3.02-3.25 (m, 4H) 3.36-3.84 (m, 8H) 4.26-4.29 (m, 2H) 6.93-6.97 (m, 3H) 7.02 (d, J=8.2 Hz, 1H) 7.24-7.30 (m, 4H)

MS (ESI+) for C₂₃H₂₇N₃O m/z 362 (M+H)⁺.

### Example 83

### Enantiomer (NB—The Chirality of the Compound is Relative)

### 6-Methoxy-1′-[2-(4-methoxyphenoxy)ethyl]-2,3,4,9-tetrahydrospiro[beta-carboline-1,3′-pyrrolidine] trifluoroacetate

6-Methoxy-2,3,4,9-tetrahydrospiro[beta-carboline-1,3′-pyrrolidine] (COMPARATIVE EXAMPLE 182, 30.0 mg, 0.12 mmol) was suspended in DMSO (200 μL) and DIPEA (114 μL, 0.65 mmol) was added. 2-(4-Methoxyphenoxy)ethyl methanesulfonate (30.2 mg, 0.12 mmol) in DMSO (400 μL) was added and the solution was stirred at 50° C. overnight. The reaction mixture was diluted with water and extracted one time with EtOAc. The organic phase was dried over Na₂SO₄, filtered and the solvent was removed at reduced pressure and the remaining oil was then diluted with MeCN (1 mL) and purified by direct injection to a preparative HPLC/UV System, MeCN:H₂O (0.1% TFA) 21-43% giving a yellow oil, 5 mg, 9%.

HPLC 96%, R_{T}=1.579 min (System A. 10-97% MeCN), 95%, R_{T}=1.424 min (System B. 10-97% MeCN).

¹H NMR (400 MHz, MeOD) δ ppm 2.57-2.69 (m, 1H) 2.70-2.76 (m, 1H) 3.05 (t, J=6.1 Hz, 2H) 3.39-3.59 (m, 4H) 3.61-3.65 (m, 2H) 3.68-3.72 (m, 1H) 3.73 (s, 3H) 3.81 (s, 3H) 3.90-3.94 (m, 1H) 4.25 (t, J=5.0 Hz, 2H) 6.83-6.86 (m, 3H) 6.89-6.92 (m, 2H) 6.98 (d, J=2.3 Hz, 1H) 7.28 (d, J=8.8 Hz, 1H)

MS (ESI+) m/z 408 (M+H)⁺.

### Example 84

### Enantiomer (NB—The Chirality of the Compound is Relative)

### 1′-[2-(4-Fluorophenoxy)ethyl]-6-methoxy-2,3,4,9-tetrahydrospiro[beta-carboline-1,3′-pyrrolidine] trifluoroacetate

6-Methoxy-2,3,4,9-tetrahydrospiro[beta-carboline-1,3′-pyrrolidine] (COMPARATIVE EXAMPLE 182, 30.0 mg, 0.12 mmol) was suspended in DMSO (200 μL) and DIPEA (114 μL, 0.65 mmol) was added. 1-(2-Bromoethoxy)-4-fluorobenzene (26.8 mg, 0.12 mmol) in DMSO (400 μL) was added and the solution was stirred at 50° C. overnight. The reaction mixture was diluted with water and extracted one time with EtOAc. The organic phase was dried over Na₂SO₄, filtered and the solvent was removed at reduced pressure and the remaining oil was then diluted with MeCN (1 mL) and purified by direct injection to a preparative HPLC/UV System, MeCN:H₂O (0.1% TFA) 22-43% giving a yellow oil 16 mg, 26%.

HPLC 97%, R_{T}=1.644 min (System A. 10-97% MeCN), 95%, R_{T}=1.477 min (System B. 10-97% MeCN).

¹H NMR (400 MHz, MeOD) δ ppm 2.64-2.71 (m, 1H) 2.75-2.82 (m, 1H) 3.06 (t, J=6.1 Hz, 2H) 3.57-3.79 (m, 7H) 3.81 (s, 3H) 4.05 (d, J=12.9 Hz, 1H) 4.32 (t, J=4.9 Hz, 2H) 6.86 (dd, J=8.8, 2.4 Hz, 1H) 6.97-7.04 (m, 5H) 7.27 (d, J=8.8 Hz, 1H)

MS (ESI+) m/z 396 (M+H)⁺.

### Example 86

### 5-[6-Methoxy-1′-(2-phenoxyethyl)-4,9-dihydrospiro[beta-carboline-1,3′-pyrrolidin]-2(3H)-yl]pentanoic acid

EXAMPLE 48 (15.8 mg, 31 mmol) was dissolved in ethanol (0.5 mL) and treated with 40% dimethylamine (xs, 500 uL). Heating at 80° C. overnight and purification with preparative HPLC produced 4.6 mg (30%) of a clear oil as a side-product to the desired amide.

HPLC 98%, R_{T}=2.55 (System A, MeCN 5-60% over 3 min). HPLC 98%, R_{T}=2.32 (System C, MeCN 5-60% over 3 min).

¹H NMR (400 MHz, CD₃OD) δ ppm 1.64 (ddd, J=14.1, 7.3, 7.2 Hz, 2H) 1.70-1.83 (m, 2H) 2.21-2.34 (m, 3H) 2.48-2.58 (m, 1H) 2.84-2.92 (m, 1H) 2.93-3.27 (m, 9H) 3.40-3.59 (m, 2H) 3.79 (s, 3H) 4.20 (t, J=5.3 Hz, 2H) 6.76 (dd, J=8.8, 2.5 Hz, 1H) 6.90-7.00 (m, 4H) 7.19 (d, J=8.8 Hz, 1H) 7.24-7.31 (m, 2H).

MS (ESI+) for C₂₈H₃₅N₃O₄ m/z 478 (M+H)⁺.

### Example 87

### Enantiomer (NB—The Chirality of the Compound is Relative)

### N-Ethyl-6-methoxy-1′-(2-phenoxyethyl)-4,9-dihydrospiro[beta-carboline-1,3′-pyrrolidine]-2(3H)-carboxamide

6-Methoxy-1′-(2-phenoxyethyl)-2,3,4,9-tetrahydrospiro[beta-carboline-1,3′-pyrrolidine] (EXAMPLE 81, 14.8 mg, 39 mmol) was mixed with ethyl isocyanate (6.0 uL, 98 mmol), pyridine (6.3 uL, 78 mmol) in dry DCM (2 mL) overnight at rt. The solvent was evaporated, the residue dissolved in MeOH with TFA (50 uL) and purified preparative HPLC (System A) to 7.9 mg clear oil (45%).

HPLC 90%, R_{T}=1.71 (System A, MeCN 30-80% over 3 min). HPLC 97%, R_{T}=1.49 (System B, MeCN 30-80% over 3 min).

MS (ESI+) for C₂₆H₃₂N₄O₃ m/z 449 (M+H)⁺.

### Example 89

### 6-Methoxy-2-[(methylsulfonyl)acetyl]-1′-(2-phenoxyethyl)-2,3,4,9-tetrahydrospiro[beta-carboline-1,3′-pyrrolidine] trifluoroacetate

6-Methoxy-1′-(2-phenoxyethyl)-2,3,4,9-tetrahydrospiro[beta-carboline-1,3′-pyrrolidine] (EXAMPLE 10, 60 mg, 0.16 mmol), N,N′-diisopropylcarbodiimide (32 μL, 0.21 mmol), (methylsulfonyl)acetic acid (29 mg, 0.21 mmol) (29 mg, 0.21 mmol) and DCM (1 mL) were shaken at ambivalent temperature for 16 h. More N,N′-diisopropylcarbodiimide (32 μL, 0.21 mmol) and (methylsulfonyl)acetic acid (29 mg, 0.21 mmol) were added to the reaction, which was shaken an additional 24 h and than the solvent was removed. The product was purified by preparative HPLC using acetonitrile-water gradients containing 0.1% triflouroacetic acid. Yield: 15.9 mg (20%).

HPLC 96%, R_{T}: 1.901 (10-97% MeCN over 3 min).

¹H NMR (270 MHz, Methanol-d₃) δ ppm 2.77-2.91 (m, 4H) 3.11 (s, 3H) 3.64-3.97 (m, 4H) 3.81 (s, 3H) 4.17-4.25 (m, 2H) 4.36-4.45 (m, 4H) 4.63 (d, J=3.46 Hz, 2H) 6.83 (dd, J=8.91, 2.47 Hz, 1H) 6.92-7.16 (m, 4H) 7.19-7.40 (m, 3H).

MS (ESI+) m/z 498 (M+H)⁺.

### Example 90

### 6-Bromo-1′-(2-phenoxyethyl)-2,3,4,9-tetrahydrospiro[beta-carboline-1,3′-pyrrolidine]

[2-(5-Bromo-1H-indol-3-yl)ethyl]amine hydrochloride (33.6 mg, 0.122 mmol) and 1-(2-phenoxyethyl)pyrrolidin-3-one (COMPARATIVE EXAMPLE 183, 25.0 mg, 0.121 mmol) were dissolved in HOAc (1 mL) and the mixture was heated at 100° C. for 1 h. The reaction mixture was then diluted with methanol (0.5 mL) and purified by direct injection to a preparative HPLC/UV System, MeCN:H₂O (0.1% TFA) 21-42% giving 10.8 mg (21%) white powder.

HPLC 100%, R_{T}=1.83 min (System A. 10-97% MeCN over 3 min), 100%, R_{T}=1.62 min (System B. 10-97% MeCN over 3 min).

¹H NMR (400 MHz, MeOD) δ ppm 2.48-2.63 (m, 3H) 3.03 (t, J=6.0 Hz, 2H) 3.09-3.26 (m, 3H) 3.47-3.66 (m, 4H) 4.24-4.26 (m, 2H) 6.93-6.96 (m, 3H) 7.25-7.31 (m, 4H) 7.65 (m, 1H).

MS (ESI+) for C₂₂H₂₄BrN₃O m/z 428 (M+H)⁺.

### Example 91

### 6-Methoxy-1′-[2-(4-methoxyphenoxy)ethyl]-N-2-thienyl-4,9-dihydrospiro[beta-carboline-1,3′-pyrrolidine]-2(3H)-carboxamide trifluoroacetate

The title compound was prepared according to the same procedure as described in EXAMPLE 81 followed by General Synthetic Procedure C affording 6.9 mg (26%) of a yellow oil.

HPLC 90%, R_{T}=3.16 (System A, MeCN 5-60% over 3 min). HPLC 90%, R_{T}=2.98 (System B, MeCN 5-60% over 3 min).

MS (ESI+) for C₂₉H₃₂N₄O₄S m/z 533 (M+H)⁺.

### Example 92

### Enantiomer (NB—The Chirality of the Compound is Relative)

### 1′-[2-(4-Chlorophenoxy)ethyl]-6-methoxy-2,3,4,9-tetrahydrospiro[beta-carboline-1,3′-pyrrolidine] trifluoroacetate

6-Methoxy-2,3,4,9-tetrahydrospiro[beta-carboline-1,3′-pyrrolidine] (COMPARATIVE EXAMPLE 182, 30.0 mg, 0.12 mmol) was suspended in DMSO (200 μL) and DIPEA (114 μL, 0.65 mmol) was added. 2-(4-Chlorophenoxy)ethyl methanesulfonate (30.7 mg, 0.12 mmol) in DMSO (400 μL) was added and the solution was stirred at 50° C. overnight. The reaction mixture was diluted with water and extracted one time with EtOAc. The organic phase was dried over Na₂SO₄, filtered and the solvent was removed at reduced pressure and the remaining oil was then diluted with MeCN (1 mL) and purified by direct injection to a preparative HPLC/UV System, MeCN:H₂O (0.1% TFA) 26-47% giving a yellow oil 10 mg, 17%.

HPLC 98%, R_{T}=1.764 min (System A. 10-97% MeCN), 95%, R_{T}=1.582 min (System B. 10-97% MeCN).

¹H NMR (400 MHz, MeOD) δ ppm 2.58-2.64 (m, 1H) 2.69-2.73 (m, 1H) 3.05 (t, J=6.0 Hz, 2H) 3.37-3.53 (m, 4H) 3.62 (t, J=6.2 Hz, 2H) 367-3.71 (m, 1H) 3.81 (s, 3H) 3.87 (d, J=12.2 Hz, 1H) 4.30 (t, J=5.0 Hz, 2H) 6.84 (dd, J=8.8, 2.4 Hz, 1H) 6.94-6.98 (m, 3H) 7.25-7.29 (m, 3H).

MS (ESI+) m/z 412 (M+H)⁺.

### Example 93

### {2-[6-Methoxy-1′-(2-phenoxyethyl)-4,9-dihydrospiro[beta-carboline-1,3′-pyrrolidin]-2(3H)-yl]-2-oxoethyl}dimethylamine trifluoroacetate

The title compound was prepared according to the General Synthetic Procedure F affording 17.8 mg (56%).

HPLC 100%, R_{T}=1.44 min (System A. 10-97% MeCN), 100%, R_{T}=1.15 min (System B. 10-90% MeCN).

¹H NMR (400 MHz, MeOD) δ ppm 2.70-2.79 (m, 1H) 2.80-3.08 (m, 9H) 3.49-3.59 (m, 1H) 3.69-4.00 (m, 8H) 4.21-4.30 (m, 1H) 4.35-4.43 (m, 2H) 4.43-4.56 (m, 3H) 6.84 (dd, J=8.78, 2.38 Hz, 1H) 6.93-7.07 (m, 4H) 7.22-7.36 (m, 3H).

MS (ESI+) m/z 463 (M+H)⁺.

HRMS (EI) calcd for C₂₇H₃₄N₄O₃: 462.2631, found 462.2631.

alt.

6-Methoxy-1′-(2-phenoxyethyl)-2,3,4,9-tetrahydrospiro[beta-carboline-1,3′-pyrrolidine] (EXAMPLE 10, 60 mg, 0.16 mmol), N,N′-diisopropylcarbodiimide (32 μL, 0.21 mmol), (dimethylamino)acetic acid (22 mg, 0.21 mmol) and DCM (1 mL) were shaken at ambivalent temperature for 16 h. More N,N′-diisopropylcarbodiimide (32 μL, 0.21 mmol) and 4-oxopentanoic acid (22 mg, 0.21 mmol) were added to the reaction, which was shaken an additional 24 h and than the solvent was removed. The product was purified by preparative HPLC using acetonitrile-water gradients containing 0.1% triflouroacetic acid. Yield: 12.1 mg (16%).

HPLC 100%, R_{T}: 1.575 (10-97% MeCN over 3 min).

¹H NMR (270 MHz, Methanol-d₃) δ ppm 2.55 (s, 6H) 2.65-2.72 (m, 4H) 2.75-2.92 (m, 4H) 3.58-3.750 (m, 4H) 3.81 (s, 3H) 4.10-4.43 (m, 6H) 6.83 (dd, J=8.66, 2.47 Hz, 1H) 6.93-7.10 (m, 4H) 7.20-7.38 (m, 3H).

MS (ESI+) m/z 463 (M+H)⁺.

### Example 94

### 1′-[2-(3-Chlorophenoxy)ethyl]-6-methoxy-2,3,4,9-tetrahydrospiro[beta-carboline-1,3′-pyrrolidine]

The title compound was prepared from COMPARATIVE EXAMPLE 1 (66 mg, 0.26 mmol) and 2-(3-chlorophenoxy)ethyl methanesulfonate (71 mg) as described in the General Synthetic Procedure B, Method B to afford 0.0274 g.

HPLC 100%, Rt=1.5989 min.

¹H-NMR (250 MHz, DMSO-d₆) δ 2.16-3.28 (m, 12H), 3.51-3.56 (s, 3H), 3.99-4.06 (s, 2H), 6.55-6.59 (d, 1H, J=8.53 Hz), 6.71-6.86 (m, 4H), 7.04-7.14 (m, 2H), 10.69-10.74 (s, 1H).

MS (ESI+) m/z 412 (M+H)⁺.

### Example 95

### 6-Methoxy-1′-[2-(2-methylphenoxy)ethyl]-2,3,4,9-tetrahydrospiro[beta-carboline-1,3′-pyrrolidine]

The title compound was prepared from COMPARATIVE EXAMPLE 1 (66 mg, 0.26 mmol) and 2-(2-methylphenoxy)ethyl methanesulfonate (65 mg) as described in the General Synthetic Procedure B, Method B to afford 0.0213 g.

HPLC 98%, Rt=1.556 min.

¹H-NMR (250 MHz, DMSO-d₆) δ 1.96-2.02 (s, 3H), 2.18-3.29 (m, 12H), 3.55-3.61 (s, 3H), 4.00-4.08 (s, 2H), 6.59-6.64 (d, 1H, J=8.53 Hz), 6.65-6.70 (m, 1H), 6.75-6.81 (m, 2H), 6.94-7.01 (m, 2H).

MS (ESI+) m/z 392 (M+H)⁺.

### Example 96

### 6-Methoxy-1′-[3-(2-methoxyphenyl)propyl]-2,3,4,9-tetrahydrospiro[beta-carboline-1,3′-pyrrolidine]

The title compound was prepared according to General Synthetic Procedure A, Method A followed by General Synthetic Procedure B, Method A.

HPLC 100%.

### Example 97

### 1′-[2-(4-Ethylphenoxy)ethyl]-6-methoxy-2,3,4,9-tetrahydrospiro[beta-carboline-1,3′-pyrrolidine]

The title compound was prepared from COMPARATIVE EXAMPLE 1 (66 mg, 0.26 mmol) and 2-(4-ethylphenoxy)ethyl methanesulfonate (69 mg) as described in the General Synthetic Procedure B, Method B to afford 0.0214 g.

HPLC 100%, Rt==1.667 min.

¹H-NMR (250 MHz, DMSO-d₆) δ 1.34-1.42 (m, 3H), 2.62-3.70 (m, 12H), 3.97-4.03 (s, 3H), 4.39-4.47 (s, 2H), 7.01-7.07 (d, 1H, J=9.14 Hz), 7.09-7.16 (d, 2H, J=7.31 Hz), 7.20-7.24 (s, 1H), 7.34-7.40 (d, 2H, J=7.92 Hz), 7-50-7.56 (d, 1H, J=8.53 Hz)

MS (ESI+) m/z 406 (M+H)⁺.

### Example 98

### 6-Methoxy-1′-(2-phenoxyethyl)-2-(piperazin-1-ylacetyl)-2,3,4,9-tetrahydrospiro[beta-carboline-1,3′-pyrrolidine] trifluoroacetate

The title compound was prepared according to the General Synthetic Procedure F affording 1.6 mg (5%).

HPLC 100%, R_{T}=1.39 min (System A. 10-97% MeCN), 98%, R_{T}=1.10 min (System B. 10-90% MeCN).

¹H NMR (400 MHz, MeOD) δ ppm 2.66-2.76 (m, 1H) 2.78-2.92 (m, 6H) 3.20-3.27 (m, 4H) 3.54-3.62 (m, 2H) 3.63-3.74 (m, 3H) 3.79-3.83 (m, 4H) 3.84-3.93 (m, 2H) 4.06-4.15 (m, 1H) 4.19-4.27 (m, 1H) 4.30-4.44 (m, 3H) 6.84 (dd, J=8.72, 2.45 Hz, 1H) 6.93-7.07 (m, 4H) 7.22-7.37 (m, 3H).

MS (ESI+) m/z 504 (M+H)⁺.

HRMS (EI) calcd for C₂₉H₃₇N₅O₃: 503.2896, found 503.2880.

### Example 99

### 2-[3-(6-Methoxy-2,3,4,9-tetrahydro-1′H-spiro[beta-carboline-1,3′-pyrrolidin]-1′-yl)propyl]hexahydro-1H-isoindole-1,3(2H)-dione

The title compound was prepared according to General Synthetic Procedure A, Method A followed by General Synthetic Procedure B, Method A.

HPLC 91%.

### Example 100

### Ethyl ({[8-methyl-1′-(2-phenoxyethyl)-4,9-dihydrospiro[beta-carboline-1,3′-pyrrolidin]-2(3H)-yl]carbonyl}amino)acetate trifluoroacetate

8-Methyl-1′-(2-phenoxyethyl)-2,3,4,9-tetrahydrospiro[β-carboline-1,3′-pyrrolidine] trifluoroacetate (EXAMPLE 124, 13.5 mg, 0.028 mmol) and ethylacetoisocyanate (5.4 mg, 0.042 mmol) were dissolved in MeCN (500 μL) and allowed to stir at rt for 1 h. The reaction mixture was then diluted with MeCN (1 mL) and purified by direct injection to a preparative HPLC/UV System, MeCN:H₂O (0.1% TFA) 28-51% giving 11.4 mg (66%) of a white powder.

HPLC 94%, R_{T}=2.18 min (System A. 10-97% MeCN over 3 min), 94%, R_{T}=1.99 min (System B. 10-97% MeCN over 3 min).

¹H NMR (400 MHz, MeOD) δ ppm 1.23 (t, J=7.1 Hz, 3H) 2.40 (s, 3H) 2.64-2.70 (m, 1H) 278-2.86 (m, 3H) 3.59-3.83 (m, 6H) 3.92 (d, J=4.6 Hz, 2H) 4.08-4.16 (m, 3H) 4.21 (d, J=12.7 Hz, 1H) 4.33-4.37 (m, 2H) 6.98-7.03 (m, 4H) 7.24-7.33 (m, 4H).

MS (ESI+) for C₂₈H₃₄N₄O₄ m/z 491 (M+H)⁺.

### Comparative Example 101

### 6-Methoxy-9-methyl-2,3,4,9-tetrahydrospiro[beta-carboline-1,3′-pyrrolidine]

EXAMPLE 126 (1.0 g, 2.88 mmol), di-tert-butyl dicarbonate (0.69 g, 3.2 mmol), DMAP (35 mg, 0.228) and were mixed in tert-butanol (50 mL) and stirred at rt overnight. The reaction mixture was heated to 40° C. and was allowed to stir for an additional 20 h. Very slow reaction. An additional amount of di-tert-butyl dicarbonate was added. The temperature was rised to 60° C. Stirring overnight. The reaction was interupted after a total time of 48 h. Very little amounts of starting material was left according to HPLC. Evaporation of solvent. The residue was extracted between DCM and 1M HCl. The organic layer was washed further with sat. NaHCO₃ (aq). Drying (Na₂SO₄), filtration and evaporation furnished a brownish oil, which was purified using flash chromatography (EtOAC:isohexane; 1:2). Evaporation of pooled fractions afforded 1.4 g of a clear, uncoloured oil. Preparative HPLC provided 130 mg of tert-butyl 1′-benzyl-6-methoxy-4,9-dihydrospiro[beta-carboline-1,3′-pyrrolidine]-2(3H)-carboxylate.

HPLC 100%, R_{T}=2.33 min (System A. 10-97% MeCN over 3 min), 100%, R_{T}=2.00 min (System B. 10-97% MeCN over 3 min).

MS (ESI+) m/z 448 (M+H)⁺.

Dry sodium hydride (3.0 mg, 0.1 mmol) was added to a precooled (0° C.) solution of the product described above dissolved in dry DMF (4 mL). Methyl iodide (6.6 μL, 0.1 mmol) was added after 10 minutes and the reaction mixture was allowed to take rt. The reaction was quenched with water after 2 h. Evaporation of solvent using SpeedVac. The product was purified by preparative HPLC (40-75%, 0.1% TFA) followed by boc-deprotection using 50% TFA/DCM at rt, for 1 h. The solvent was evaporated and the deprotected compound was purified further by preparative HPLC (15-45%, 0.1% TFA). GeneVac of pooled fraction afforded product in 98% yield, which was subjected to further deprotection by debenzylation using ammonium formiate (9.0 mg, 0.1 mmol) and a catalytic amount of 10% Pd/C. The reaction was run in methanol (3 mL) in microwave oven for 140° C. in 180 s. The mixture was filtered followed by evaporation of solvent. The crude product was purified by preparative HPLC (7-25%, 0.1% TFA). Acetonitrile was evaporated and the water layer was extracted with DCM. The organic layer was washed with sat. NaHCO₃ (aq). Drying (Na₂SO₄), filtration and evaporation afforded 20 mg (78%) of the title compound.

HPLC 99%, R_{T}=1.55 min (System A. 10-97% MeCN over 3 min), 99%, R_{T}=0.94 min (System B. 10-97% MeCN over 3 min).

¹H NMR (400 MHz, CHLOROFORM-D) δ ppm 1.8 (m, 1H) 2.3 (m, 1H) 2.7 (t, J=5.6 Hz, 2H) 3.1 (m, 5H) 3.4 (m, 1H) 3.7 (s, 3H) 3.8 (s, 3H) 6.8 (m, 2H) 7.1 (d, J=8.8 Hz, 1H).

MS (ESI+) m/z 272 (M+H)⁺.

### Example 102

### N-(2-Methoxyethyl)-2-[6-methoxy-1′-(2-phenoxyethyl)-4,9-dihydrospiro[beta-carboline-1,3′-pyrrolidin]-2(3H)-yl]-2-oxoethanamine trifluoroacetate

The title compound was prepared according to the General Synthetic Procedure F affording 6.5 mg (16%).

HPLC 92%, R_{T}=1.56 min (System A. 10-97% MeCN), 92%, R_{T}=2.02 min (System B. 5-60% MeCN).

¹H NMR (400 MHz, MeOD) δ ppm 2.69-2.79 (m, 1H) 2.80-2.92 (m, 3H) 3.26-3.34 (m, 2H) 3.41 (s, 3H) 3.52-3.61 (m, 1H) 3.63-3.68 (m, 2H) 3.72-3.78 (m, 1H) 3.80-3.83 (m, 4H) 3.84-3.96 (m, 3H) 4.21-4.50 (m, 6H) 6.84 (dd, J=8.78, 2.38 Hz, 1H) 6.95-7.05 (m, 4H) 7.26-7.34 (m, 3H).

MS (ESI+) m/z 493 (M+H)⁺.

HRMS (EI) calcd for C₂₈H₃₆N₄O₂: 492.2737, found 492.2734.

### Example 103

### ({[6-Methoxy-1′-(2-phenoxyethyl)-4,9-dihydrospiro[beta-carboline-1,3′-pyrrolidin]-2(3H)-yl]carbonyl}amino)acetic acid

A dry acetonitrile solution of 6-methoxy-1′-(2-phenoxyethyl)-2,3,4,9-tetrahydrospiro[beta-carboline-1,3′-pyrrolidine] (EXAMPLE 10, 25 mg in 1 mL, 66 mmol) was treated with pyridine (7.1 uL, 87 mmol) and ethylacetoisocyanate (8.9 uL, 80 mmol) and mixed at rt for 1 h. NaOH (250 uL, 1M) is added and the solution stirred at rt for 2 h. Purification by HPLC (System B) to 21.6 mg white solid/gum (68%).

HPLC 95%, R_{T}=2.58 (System A, MeCN 5-60% over 3 min). HPLC 95%, R_{T}=2.41 (System B, MeCN 5-60% over 3 min).

¹H NMR (400 MHz, CD₃OD) δ ppm 2.02-2.03 (m, 3H) 2.43-2.51 (m, 1H) 2.74 (s, 3H) 3.10-3.20 (m, 2H) 3.45-3.50 (m, 1H) 3.54-3.63 (m, 1H) 3.76-3.83 (m, 2H) 3.80 (s, 3H) 3.89-3.96 (m, 1H) 4.20-4.28 (m, 2H) 6.75 (dd, J=8.8, 2.5 Hz, 1H) 6.89-7.00 (m, 4H) 7.21 (d, J=9.0 Hz, 1H) 7.24-7.31 (m, 2H).

MS (ESI+) for C₂₆H₃₀N₄O₅ m/z 479 (M+H)⁺.

### Example 104

### Methyl 3-[6-methoxy-1′-(2-phenoxyethyl)-4,9-dihydrospiro[beta-carboline-1,3′-pyrrolidin]-2(3H)-yl]-3-oxopropanoate trifluoroacetate

6-Methoxy-1′-(2-phenoxyethyl)-2,3,4,9-tetrahydrospiro[beta-carboline-1,3′-pyrrolidine] (EXAMPLE 10, 50 mg, 0.1 mmol), triethylamine (18 μL, 0.1 mmol), methyl 3-chloro-3-oxopropanoate (14 μL, 0.1 mmol) and acetonitrile (1 mL) were shaken at ambivalent temperature for 1 h and the product was purified by preparative HPLC using acetonitrile-water gradients containing 0.1% triflouroacetic acid. Yield: 1.95 mg (4%).

HPLC 97%, R_{T}: 2.100 (10-97% MeCN over 3 min).

¹H NMR (270 MHz, Methanol-d₃) δ ppm 2.64-2.92 (m, 4H) 3.56-3.70 (m, 2H) 3.72 (s, 3H) 3.76 (d, J=13.61 Hz, 2H) 3.81 (s, 3H) 3.82-4.06 (m, 4H) 4.16-4.40 (m, 4H) 6.83 (dd, J=8.91, 2.47 Hz, 1H) 6.96-7.03 (m, 4H) 7.27-7.32 (m, 3H).

MS (ESI+) m/z 478 (M+H)⁺.

### Example 105

### 4-[6-Methoxy-1′-(2-phenoxyethyl)-4,9-dihydrospiro[beta-carboline-1,3′-pyrrolidin]-2(3H)-yl]-4-oxobutanoic acid trifluoroacetate

6-Methoxy-1′-(2-phenoxyethyl)-2,3,4,9-tetrahydrospiro[beta-carboline-1,3′-pyrrolidine] (EXAMPLE 10, 60 mg, 0.16 mmol), N,N′-diisopropylcarbodiimide (32 μL, 0.21 mmol), succinic acid (45 mg, 0.21 mmol) and DCM (1 mL) were shaken at ambivalent temperature for 16 h. More N,N′-diisopropylcarbodiimide (32 μL, 0.21 mmol) and 4-oxopentanoic acid (25 mg, 0.21 mmol) were added to the reaction, which was shaken an additional 24 h and than the solvent was removed. The product was purified by preparative HPLC using acetonitrile-water gradients containing 0.1% triflouroacetic. Yield: 5.2 mg (7%).

HPLC 100%, R_{T}: 1.939 (10-97% MeCN over 3 min).

¹H NMR (270 MHz, Methanol-d₃) δ ppm 2.76-3.00 (m, 8H) 3.71-3.99 (m, 4H) 3.81 (s, 3H) 4.38-4.41 (m, 2H) 4.46-4.48 (m, 2H) 4.53-4.54 (m, 2H) 6.84 (dd, J=8.91, 2.47 Hz, 1H) 6.92-7.09 (m, 4H) 7.23-7.37 (m, 3H).

MS (ESI+) m/z 478 (M+H)⁺.

### Example 106

### N-(3,5-Dimethylisoxazol-4-yl)-6-methoxy-1′-[2-(4-methoxyphenoxy)ethyl]-4,9-dihydrospiro[beta-carboline-1,3′-pyrrolidine]-2(3H)-carboxamide trifluoroacetate

10.7 mg (39%) of a yellow oil was prepared according to the same procedure as described in EXAMPLE 81 followed by General Synthetic Procedure C.

HPLC 92%, R_{T}=2.88 (System A, MeCN 5-60% over 3 min). HPLC 92%, R_{T}=2.69 (System B, MeCN 5-60% over 3 min).

¹H NMR (400 MHz, CD₃OD) δ ppm 2.11 (s, 3H) 2.26 (s, 3H) 2.67-2.77 (m, 1H) 2.79-2.88 (m, 1H) 2.87-2.94 (m, 2H) 3.60-3.69 (m, 2H) 3.70-3.78 (m, 3H) 3.74 (s, 3H) 3.81 (s, 3H) 3.93-4.04 (m, 1H) 4.10-4.19 (m, 1H) 4.21-4.32 (m, 3H) 6.80-6.83 (m, 4H) 6.83-6.85 (m, 1H) 6.97 (d, J=2.5 Hz, 1H) 7.27 (d, J=8.8 Hz, 1H).

MS (ESI+) for C₃₀H₃₅N₅O₅ m/z 546 (M+H)⁺.

### Example 107

### {2-[6-Methoxy-1′-(2-phenoxyethyl)-4,9-dihydrospiro[beta-carboline-1,3′-pyrrolidin]-2(3H)-yl]-2-oxoethyl}amine trifluoroacetate

The title compound was prepared according to the General Synthetic Procedure F affording 8.4 mg (28%).

HPLC 100%, R_{T}=1.43 min (System A. 10-97% MeCN), 100%, R_{T}=1.13 min (System B. 10-90% MeCN).

¹H NMR (400 MHz, MeOD) δ ppm 2.69-2.92 (m, 4H) 3.53-3.64 (m, 1H) 3.72-3.96 (m, 8H) 4.15-4.27 (m, 3H) 4.35-4.48 (m, 3H) 6.84 (dd, J=8.85, 2.32 Hz, 1H) 6.93-7.06 (m, 4H) 7.23-7.35 (m, 3H).

MS (ESI+) m/z 435 (M+H)⁺.

HRMS (EI) calcd for C₂₅H₃₀N₄O₃: 434.2318, found 434.2332.

### Example 108

### {2-[6-Methoxy-1′-(2-phenoxyethyl)-4,9-dihydrospiro[beta-carboline-1,3′-pyrrolidin]-2(3H)-yl]-2-oxoethyl}methylamine trifluoroacetate

The title compound was prepared according to the General Synthetic Procedure F affording 10.5 mg (34%).

HPLC 100%, R_{T}=1.44 min (System A. 10-97% MeCN), 100%, R_{T}=1.14 min (System B. 10-90% MeCN).

¹H NMR (400 MHz, MeOD) δ ppm 2.70-2.93 (m, 7H) 3.52-3.63 (m, 1H) 3.71-3.96 (m, 8H) 4.19-4.33 (m, 3H) 4.34-4.49 (m, 3H) 6.84 (dd, J=8.78, 2.38 Hz, 1H) 6.94-7.06 (m, 4H) 7.25-7.35 (m, 3H).

MS (ESI+) m/z 449 (M+H)⁺.

HRMS (EI) calcd for C₂₆H₃₂N₄O₃: 448.2474, found 448.2460.

### Example 109

### 7-Methoxy-14-oxo-16-(2-phenoxyethyl)-3,13-diaza-16-azoniapentacyclo[14.2.1.0˜1,13˜.0˜2,10˜.0˜4,9˜]nonadeca-2(10),4,6,8-tetraene chloride

A solution of 2-(chloroacetyl)-6-methoxy-1′-(2-phenoxyethyl)-2,3,4,9-tetrahydrospiro[beta-carboline-1,3′-pyrrolidine] (0.20 g 4.4 mmol) prepared as described in General Synthetic Procedure D, Method A was dissolved in MeOH and evaporated at reduced pressure with about 60° C. in the water bath. The remaining solid crisp was trituated with a small amount of CHCl₃ to give a white precipitate that was dried (60° C. 10 mmHg) overnight to give 0.10 g (2.2 mmol, 50%) of the target molecule.

HPLC 100%, R_{T}=1.68 min (System A. 10-97% MeCN), 100%, R_{T}=1.40 min (System B. 10-90% MeCN).

¹H NMR (400 MHz, DMSO-D6) δ ppm 2.57-2.70 (m, 1H) 2.70-2.81 (m, 1H) 2.82-2.91 (m, 1H) 2.95-3.12 (m, 2H) 3.75 (s, 3H) 3.91-4.03 (in, J=14.31 Hz, 1H) 4.05-4.27 (m, 3H) 4.28-4.42 (m, 1H) 4.50-4.68 (m, 4H) 4.72 (dd, J=13.05, 4.77 Hz, 1H) 4.95 (d, J=10.79 Hz, 1H) 6.78 (dd, J=8.78, 2.26 Hz, 1H) 6.94-7.07 (m, 4H) 7.27 (d, J=8.78 Hz, 1H) 7.33 (t, J=7.91 Hz, 2H) 11.67 (s, 1H).

¹³C NMR (DMSO-d₆) δ 20.18, 36.73, 37.64, 55.37, 60.98, 61.52, 62.97, 63.28, 63.84, 66.47, 100.18, 110.01, 112.21, 112.36, 114.74, 121.40, 125.93, 127.59, 129.57, 131.35, 153.44, 157.29, 160.23.

MS (ESI+) m/z 418.

HRMS (EI) calcd for C₂₅H₂₈N₃O₃: 418.2131, found 418.2121.

### Example 110

### N2-{2-[6-Methoxy-1′-(2-phenoxyethyl)-4,9-dihydrospiro[beta-carboline-1,3′-pyrrolidin]-2(3H)-yl]-2-oxoethyl}glycinamide trifluoroacetate-N,N-diethylethanamine (1:1)

The title compound was prepared according to the General Synthetic Procedure F affording 0.9 mg (2%).

HPLC 100%, R_{T}=1.48 min (System A. 10-97% MeCN), 95%, R_{T}=1.91 min (System B. 5-60% MeCN).

¹H NMR (400 MHz, MeOD) δ ppm 2.71-2.93 (m, 4H) 3.53-3.63 (m, 1H) 3.66-3.97 (m, 10H) 4.20-4.49 (m, 6H) 6.85 (dd, J=8.78, 2.38 Hz, 1H) 6.95-7.06 (m, 4H) 7.25-7.37 (m, 3H).

MS (ESI+) m/z 492 (M+H)⁺.

HRMS (EI) calcd for C₂₇H₃₃N₅O₄: 491.2533, found 491.2529.

### Example 111

### 6-Methoxy-1′-[2-(phenylthio)ethyl]-2,3,4,9-tetrahydrospiro[beta-carboline-1,3′-pyrrolidine] trifluoroacetate

6-Methoxy-2,3,4,9-tetrahydrospiro[beta-carboline-1,3′-pyrrolidine (COMPARATIVE EXAMPLE 1, 25 mg, 0,097 mmol) was suspended in DMSO (75 μL) and DIPEA (57.8 mg, 0.447 mmol) was added. 2-Bromoethyl phenyl sulfide (27.42 mg, 0.126 mmol) in DMSO (150 μL) was added and the solution was shaken at rt overnight. The reaction mixture was then diluted with methanol and purified by direct injection to a preparative HPLC/UV System, MeCN:H₂O (0.1% TFA) 19-40% giving 24.7 mg (65%) of product.

HPLC 99%, R_{T}=1.69 min (System A. 10-97% MeCN over 3 min).

¹H NMR (400 MHz, MeOD) δ ppm 2.49-2.62 (m, 2H) 3.02-3.10 (m, 6H) 3.23-3.26 (m, 3H) 3.43-3.48 (m, 2H) 3.60 (t, J=5.9 Hz, 2H) 3.80 (s, 3H) 6.83 (dd, J=8.8, 2.4 Hz, 1H) 6.96 (d, J=2.3 Hz, 1H) 7.20-7.26 (m, 2H) 7.32 (t, J=7.7 Hz, 2H) 7.41 (d, J=7.5 Hz, 2H).

MS (ESI+) for C₂₃H₂₇N₃OS m/z 394 (M+H)⁺.

### Example 112

### 6-Methoxy-2-(morpholin-4-ylacetyl)-1′-(2-phenoxyethyl)-2,3,4,9-tetrahydrospiro[beta-carboline-1,3′-pyrrolidine] trifluoroacetate

The title compound was prepared according to the General Synthetic Procedure F affording 10.3 mg (30%).

HPLC 100%, R_{T}=1.45 min (System A. 10-97% MeCN), 100%, R_{T}=1.16 min (System B. 10-90% MeCN).

The morpholine ring gives broad signals.

¹H NMR (400 MHz, MeOD) δ ppm 2.71-2.80 (m, 1H) 2.81-2.98 (m, 3H) 3.32-3.67 (m, 5H) 3.69-4.16 (m, 12H) 4.21-4.30 (m, 1H) 4.34-4.44 (m, 2H) 4.44-4.60 (m, 3H) 6.84 (dd, J=8.85, 2.45 Hz, 1H) 6.94-7.06 (m, 4H) 7.24-7.38 (m, 3H).

MS (ESI+) m/z 505 (M+H)⁺.

HRMS (EI) calcd for C₂₉H₃₆N₄O₄: 504.2737, found 504.2725.

### Example 113

### 1′-[2-(Benzyloxy)ethyl]-6-methoxy-2,3,4,9-tetrahydrospiro[beta-carboline-1,3′-pyrrolidine] trifluoroacetate

6-Methoxy-2,3,4,9-tetrahydrospiro[beta-carboline-1,3′-pyrrolidine] hydrochloride (COMPARATIVE EXAMPLE 1, 0.050 g, 0.194 mmol), [(2-bromoethoxy)methyl]benzene (0.054 g, 0.253 mmol) and K₂CO₃ (0.035 g, 0.253 mmol) in MeCN (2 mL) and a few drops of DMF was heated with stirring to 70° C. until no more product was formed according to LC-MS (1 week). The reaction mixture was filtered and purified using preparative LC (System A, 20-50% MeCN over 5 min) affording 0.0113 g (15%) of the wanted product as orange gum.

HPLC 99% R_{T}=1.64 min (System A. 10-97% MeCN over 3 min), 100% R_{T=}1.44 min (System B. 10-97% MeCN over 3 min).

¹H NMR (270 MHz, CHLOROFORM-D) δ ppm 2.53 (d, J=2.23 Hz, 2H) 2.81-3.03 (m, 3H) 3.45 (d, 8H) 3.76 (s, 3H) 3.79-3.97 (m, 1H) 4.27-4.49 (m, 2H) 6.72-6.93 (m, 2H) 7.16-7.38 (m, 6H) 10.92 (s, 1H).

MS (ESI+) for C₂₄H₂₉N₃O₂ m/z 392 (M+H)⁺.

### Example 114

### {3-[6-Methoxy-1′-(2-phenoxyethyl)-4,9-dihydrospiro[beta-carboline-1,3′-pyrrolidin]-2(3H)-yl]-1,1-dimethyl-3-oxopropyl}amine hydrochloride

6-Methoxy-1′-(2-phenoxyethyl)-2,3,4,9-tetrahydrospiro[beta-carboline-1,3′-pyrrolidine] (EXAMPLE 10, 60 mg, 0.16 mmol), N,N′-diisopropylcarbodiimide (32 μL, 0.21 mmol), 3-[(tert-butoxycarbonyl)amino]-3-methylbutanoic acid (45 mg, 0.21 mmol) and DCM (1 mL) were shaken at ambivalent temperature for 16 h. More N,N′-diisopropylcarbodiimide (32 μL, 0.21 mmol) and 3-[(tert-butoxycarbonyl)amino]-3-methylbutanoic acid (45 mg, 0.21 mmol) were added to the reaction, which was shaken an additional 24 h and than the solvent was removed. The product was purified by preparative HPLC using acetonitrile-water gradients containing 0.1% triflouroacetic acid and deprotected with 2 M HCl in diethyl ether. Yield: 10.2 mg (13%).

HPLC 100%, R_{T}: 1.687 (10-97% MeCN over 3 min).

1H NMR (270 MHz, Methanol-d₃) δ ppm 2.86 (d, J=2.97 Hz, 4H) 3.01 (d, J=2.97 Hz, 2H) 3.34 (s, 6H) 3.59-3.64 (m, 2H) 3.81 (s, 3H) 3.88-4.05 (m, 6H) 4.42-4.45 (m, 2H) 6.83 (dd, J=8.78, 2.35 Hz, 1H) 6.93-7.09 (m, 4H) 7.31 (t, J=7.92 Hz, 3H).

MS (ESI+) m/z 477 (M+H)⁺.

### Example 115

### 7-Methoxy-1′-(2-phenoxyethyl)-2,3,4,9-tetrahydrospiro[beta-carboline-1,3′-pyrrolidine] trifluoroacetate

The title compound was prepared according to General Synthetic Procedure A, Method C. Preparative HPLC/UV System, MeCN:H₂O (0.1% TFA) 16-37% afforded 68 mg (88%) of product.

HPLC 99%, R_{T}=1.57 min (System A. 10-97% MeCN over 3 min), 100%, R_{T}=1.39 min (System B. 10-97% MeCN over 3 min).

¹H NMR (400 MHz, MeOD) δ ppm 2.71-2.91 (m, 2H) 3.05 (t, J=6.1 Hz, 2H) 3.62-3.75 (m, 5H) 3.81 (s, 3H) 3.89-3.99 (m, 2H) 4.2 (d, J=13.6 Hz, 1H) 4.4 (t, J=4.9 Hz, 2H) 6.8 (dd, J=8.7, 2.2 Hz, 1H) 6.9 (d, J=2.1 Hz, 1H) 6.96-7.00 (m, 3H) 7.27-7.31 (m, 2H) 7.4 (d, J=8.7 Hz, 1H)

MS (ESI+) for C₂₃H₂₇N₃O₂ m/z 378 (M+H)⁺.

### Example 116

### 1′-[4-(Difluoromethoxy)benzyl]-6-methoxy-2,3,4,9-tetrahydrospiro[beta-carboline-1,3′-pyrrolidine]

The title compound was prepared according to General Synthetic Procedure A, Method A followed by General Synthetic Procedure B, Method A.

HPLC 94%.

### Example 117

### 1′-[2-(1H-Indol-3-yl)ethyl]-6-methoxy-2,3,4,9-tetrahydrospiro[beta-carboline-1,3′-pyrrolidine] trifluoroacetate

6-Methoxy-2,3,4,9-tetrahydrospiro[beta-carboline-1,3′-pyrrolidine] hydrochloride (COMPARATIVE EXAMPLE 1, 0.020 g, 0.078 mmol) was suspended in DMSO (0.15 mL) and DIPEA (4.6 eq, 0.062 mL) was added. 3-(2-Bromoethyl)-1H-indole (0.023 g, 0.101 mmol) dissolved in DMSO (0.30 mL) was added. The reaction mixture was agitated at room temperature until no more product was formed according to LC-MS (5 days).

The reaction mixture was dilutet with MeCN and purification was performed using preparative LC (System A, 15-45% MeCN over 5 min) affording 0.0091 g (29%) of a white solid.

HPLC 94% R_{T}=1.59 min (System A. 10-97% MeCN over 3 min), 97% R_{T}=1.42 min (System B. 10-97% MeCN over 3 min).

¹H NMR (270 MHz, METHANOL-D3) δ ppm 1.33-1.41 (m, 1H) 2.63-2.71 (m, 4H) 2.78-2.93 (m, 1H) 2.98 (t, J=8.41 Hz, 1H) 3.06 (t, J=5.94 Hz, 2H) 3.51-3.78 (m, 4H) 3.80 (s, 3H) 4.04 (s, 1H) 6.85 (dd, J=8.91, 2.35 Hz, 1H) 6.96-7.14 (m, 3H) 7.23-7.27 (m, 1H) 7.33 (t, J=8.78 Hz, 2H) 7.67 (d, J=7.42 Hz, 1H)

MS (ESI+) for C₂₅H₂₈N₄₀ m/z 401 (M+H)⁺.

### Example 118

### 6-Methoxy-2-(2-morpholin-4-ylethyl)-1′-(2-phenoxyethyl)-2,3,4,9-tetrahydrospiro[beta-carboline-1,3′-pyrrolidine]

The title compound was prepared according to General Synthetic Procedure E, Method A using 50 μmol of EXAMPLE 10 at 80° C. over the weekend. 1.7 mg (7%) of a clear oil was produced.

HPLC 90%, R_{T}=2.55 (System A, MeCN 5-60% over 3 min). HPLC 90%, R_{T}=2.31 (System B, MeCN 5-60% over 3 min).

MS (ESI+) for C₂₉H₃₈N₄O₃ m/z 491 (M+H)⁺.

### Example 119

### 6-Methoxy-1′-(2-phenoxyethyl)-2-(pyridin-2-ylmethyl)-2,3,4,9-tetrahydrospiro[beta-carboline-1,3′-pyrrolidine]

The title compound was prepared according to General Synthetic Procedure E, Method A using 50 μmol of EXAMPLE 10 at rt overnight. 0.5 mg (2%) of a clear oil was produced.

HPLC 98%, R_{T}=2.44 (System A, MeCN 5-60% over 3 min). HPLC 98%, R_{T}=2.22 (System B, MeCN 5-60% over 3 min).

¹H NMR (400 MHz, CD₃OD) δ ppm 2.54 (s, 2H) 3.12 (dt, J=3.3, 1.6 Hz, 1H) 3.44-3.53 (m, 3H) 3.66-3.76 (m, 2H) 3.81 (s, 3H) 3.86-3.95 (m, 1H) 4.06-4.16 (m, 2H) 4.21-4.31 (m, 1H) 4.40 (t, J=5.1 Hz, 2H) 6.80 (dd, J=8.8, 2.4 Hz, 1H) 6.80 (dd, J=8.8, 2.4 Hz, 1H) 6.92-7.05 (m, 4H) 7.24 (d, J=8.7 Hz, 1H) 7.27-7.34 (m, 2H) 7.40-7.47 (m, 1H) 7.55 (d, J=9.2 Hz, 1H) 7.88-7.94 (m, 1H) 8.67 (d, J=4.3 Hz, 1H) 8.67 (d, J=4.3 Hz, 1H).

MS (ESI+) for C₂₉H₃₂N₄O₂ m/z 469 (M+H)⁺.

### Example 120

### 6-Methoxy-1′-(3-phenoxypropyl)-2,3,4,9-tetrahydrospiro[beta-carboline-1,3′-pyrrolidine]

The title compound was prepared according to General Synthetic Procedure A, Method A followed by General Synthetic Procedure B, Method A.

HPLC 100%.

### Example 121

### Enantiomer (NB—The Chirality of the Compound is Relative)

6-Methoxy-1′,2-bis(2-phenoxyethyl)-2,3,4,9-tetrahydrospiro[beta-carboline-1,3′-pyrrolidine]

2.0 mg (4%) of an yellow oil was obtained as a side-product in the synthesis of EXAMPLE 81.

HPLC 100%, R_{T}=2.12 (System A, MeCN 30-80% over 3 min). HPLC 100%, R_{T}=3.02 (System C, MeCN 30-80% over 3 min).

¹H NMR (400 MHz, CHLOROFORM-D) δ ppm 1.06-1.41 (m, 2H) 1.65-1.76 (m, 1H) 1.86-2.00 (m, J=16.3 Hz, 1H) 2.89-2.98 (m, 1H) 2.99-3.10 (m, 1H) 3.34-3.54 (m, 3H) 3.55-3.73 (m, 3H) 3.84 (s, 3H) 3.95-4.16 (m, 2H) 4.26-4.38 (m, 3H) 4.38-4.46 (m, 1H) 6.86-6.94 (m, 6H) 6.97-7.04 (m, 2H) 7.27-7.35 (m, 5H).

MS (ESI+) for C₃₁H₃₅N₃O₃ m/z 498 (M+H)⁺.

### Example 122

### 6-Methoxy-1′-{2-[4-(methylsulfonyl)phenoxy]ethyl}-2,3,4,9-tetrahydrospiro[beta-carboline-1,3′-pyrrolidine]

6-Methoxy-2,3,4,9-tetrahydrospiro[beta-carboline-1,3′-pyrrolidine (COMPARATIVE EXAMPLE 1, 45 mg, 0.175 mmol) was mixed with DMF (0.9 mL) in a vial and K₂CO₃ (27 mg, 0.2 mmol) was added. The mesylate (42 mg) prepared from 2-[4-(methylsulfonyl)phenoxy]ethanol was added. The mixtures were heated to 70° C. for 4 h and then left at rt overnight. Heating was continued for another 3 h. Water and acetic acid was added and the solvent was removed in a Genevac. The crude product were purified by preparatory HPLC on an YMC-column with acetonitrile −0.1% TFA in water as eluent affording 38 mg of the title compound as TFA-salt.

HPLC 100%, R_{T}=1.46 min (System A, 10-97% MeCN over 3 min).

¹H NMR (270 MHz, CD₃OD) δ 2.58-2.87 (m, 2H), 3.00-3.11 (m, 5H), 3.49-3.74 (m, 6H), 3.77-3.86 (m, 4H), 4.05 (d, J=12.9 Hz, 1H), 4.46 (m, 2H), 6.85 (dd, J=2.5, 8.9 Hz, 1H), 6.99 (d, J=2.3 Hz, 1H), 7.11-7.24 (m, 2H), 7.28 (d, J=8.7 Hz, 1H), 7.83-7.93 (m, 2H).

MS (ESI+) m/z 456 (M+H)⁺.

### Comparative Example 123

### 6-Methoxy-2-(2-phenoxyethyl)-2,3,4,9-tetrahydrospiro[beta-carboline-1,3′-pyrrolidine]

tert-Butyl 6-methoxy-2,3,4,9-tetrahydro-1′H-spiro[beta-carboline-1,3′-pyrrolidine]-1′-carboxylate (COMPARATIVE EXAMPLE 202, 100 mg, 0.28 mmol) was dissolved in dry DMF (1 mL) and treated with DIPEA (98 μL, 0.56 mmol) and β-bromophenetole (93 mg, 0.46 mmol) at 100° C. for 26 h. The crude product was dissolved in TFA (100 μL), water (400 μL) and MeOH (200 uL), filtered and purified with preparative HPLC (System A) to 78.5 mg light brown solid. The products was taken up in a few mL 1/1 DCM/TFA and stirred at rt for 1 h. The solvent was evaporated to 72 mg yellow gum (52% in two steps).

HPLC 97%, R_{T}=1.56 (System C, MeCN 30-80% over 3 min).

¹H NMR (400 MHz, CD₃OD) δ ppm 2.65-2.76 (m, 1H) 2.80-2.92 (m, 2H) 3.08-3.20 (m, 1H) 3.33-3.48 (m, 2H) 3.55 (d, J=13.2 Hz, 1H) 3.59-3.87 (m, 4H) 3.81 (s, 3H) 4.08 (d, J=13.2 Hz, 1H) 4.20-4.36 (m, 2H) 6.82 (dd, J=8.8, 2.5 Hz, 1H) 6.94-7.02 (m, 4H) 7.24-7.33 (m, 3H).

MS (ESI+) for C₂₃H₂₇N₃O₂ m/z 378 (M+H)⁺.

### Example 124

### 8-Methyl-1′-(2-phenoxyethyl)-2,3,4,9-tetrahydrospiro[beta-carboline-1,3′-pyrrolidine]

The title compound was prepared according to General Synthetic Procedure A, Method C. Preparative HPLC/UV System, MeCN:H₂O (0.1% TFA) 18-40% afforded 21.4 mg (48%) white powder.

HPLC 97%, R_{T}=1.71 min (System A. 10-97% MeCN over 3 min), 100%, R_{T}=1.50 min (System B. 10-97% MeCN over 3 min).

¹H NMR (400 MHz, MeOD) δ ppm 2.45-2.49 (m, 1H) 2.5 (s, 3H) 2.62-2.70 (m, 1H) 3.03-3.25 (m, 4H) 3.46-3.58 (m, 6H) 4.23-4.28 (m, 2H) 6.93-6.98 (m, 5H) 7.26-7.32 (m, 3H).

MS (ESI+) for C₂₃H₂₇N₃O m/z 362 (M+H)⁺.

### Example 125

### 4-{2-[6-Methoxy-1′-(2-phenoxyethyl)-4,9-dihydrospiro[beta-carboline-1,3′-pyrrolidin]-2(3H)-yl]-2-oxoethyl}piperazin-2-one trifluoroacetate

The title compound was prepared according to the General Synthetic Procedure F affording 8.1 mg (19%).

HPLC 99%, R_{T}=1.48 min (System A. 10-97% MeCN), 99%, R_{T}=2.91 min (System B. 5-60% MeCN).

¹H NMR (400 MHz, MeOD) δ ppm 2.70-2.79 (m, 1H) 2.81-2.95 (m, 3H) 3.38-3.48 (m, 2H) 3.49-3.62 (m, 3H) 3.69-3.98 (m, 10H) 4.20-4.53 (m, 6H) 6.84 (dd, J=8.85, 2.45 Hz, 1H) 6.96-7.06 (m, 4H) 7.26-7.36 (m, 3H).

MS (ESI+) m/z 518 (M+H)⁺.

HRMS (EI) calcd for C₂₉H₃₅N₅O₄: 517.2689, found 517.2697.

### Example 126

### 1′-Benzyl-6-methoxy-2,3,4,9-tetrahydrospiro[beta-carboline-1,3′-pyrrolidine] hydrochloride

The title compound was prepared according to General Synthetic Procedure A, Method B but without the subsequent debenzylation step. Flash chromatography afforded 9.80 g (28.2 mmol, 90.1%) of a brown oil that crystallized upon standing. An analytical sample was precipitated as its hydrochloride salt with HCl/ether to give grey crystalline solid.

HPLC 100%, R_{T}=1.86 min (System A. 5-60% MeCN), 100%, R_{T}=1.02 min (System B. 10-90% MeCN).

M.p. for the free base=118.5° C., for the HCl salt=181.5-183° C.

NMR for the free base; ¹H NMR (400 MHz, CHLOROFORM-D) δ ppm 2.00-2.11 (m, 1H) 2.23-2.35 (m, 1H) 2.44 (d, J=8.78 Hz, 1H) 2.58 (q, J=8.95 Hz, 1H) 2.67 (dd, J=6.53, 4.77 Hz, 2H) 3.04-3.15 (m, 3H) 3.16-3.25 (m, 1H) 3.64-3.75 (m, 2H) 3.84 (s, 3H) 6.80 (dd, J=8.66, 2.38 Hz, 1H) 6.92 (d, J=2.51 Hz, 1H) 7.19-7.29 (m, 2H) 7.30-7.40 (m, 4H) 8.75 (s, 1H)

¹³C NMR (CDCl₃) δ ppm 22.54, 39.43, 41.79, 53.00, 55.97, 59.81, 60.12, 66.35, 100.42, 106.19, 111.06, 111.47, 127.29, 127.34, 128.43, 128.76, 130.44, 138.11, 141.24, 153.85.

HRMS (EI) calcd for C₂₂H₂₅N₃O: 347.1998, found 347.1983.

Elemental analysis calc for C₂₂H₂₅N₃O.2HCl (C, H, N.)

Chiral Separation:

EXAMPLE 126 (35.8 g, 0.1 mol) was dissolved in 120 mL of refluxing methanol and N-acetyl L-phenylalanine (22.8 g, 0.11 mol) dissolved in 80 mL of hot methanol was added. After 16 h the precipitate was filtered off and washed with ethanol. The mother liquor was concentrated and another crop was obtained. Both crops was recrystallized from methanol and washed with ethanol giving 19.8 g salt of the first enantiomer.

The combined mother liquors from the two crops were concentrated and the free base was extracted with ethyl acetate from a potassium aqueous carbonate solution. After drying and concentration, 13 g of oil was obtained. The oil was dissolved in 100 mL of hot ethanol and N-acetyl D-phenylalanine (7.8 g, 0.037 mol) in 100 mL of hot ethanol was added. After 16 h the precipitate was filtered off and washed with ethanol giving a first crop of the second enantiomer. After a short series of crystallizations and extractions, 25.6 g of the salt of the first enantiomer and 22.2 g of the salt of the second enantiomer was obtained.

The hydrochloride salt of the enantiomers was prepared by extracting the free base from an aqueous potassium carbonate solution with ethyl acetate. Then the hydrochloride was precipitated from an ether solution with hydrogen chloride in ether.

This procedure gave 19.2 g of the hydrochloride of the first enantiomer with an optical purity of 97% and 16.9 g of the second enantiomer with an optical purity of 100%; [α]_{D} −27.0° (c=2.9 MeOH).

### Example 127

### ({2-[6-Methoxy-1′-(2-phenoxyethyl)-4,9-dihydrospiro[beta-carboline-1,3′-pyrrolidin]-2(3H)-yl]-2-oxoethyl}amino)acetic acid trifluoroacetate

To a solution of ethyl ({2-[6-methoxy-1′-(2-phenoxyethyl)-4,9-dihydrospiro[beta-carboline-1,3′-pyrrolidin]-2(3H)-yl]-2-oxoethyl}amino)acetate trifluoroacetate (EXAMPLE 54, 10 mg 16 μmol) in MeOH (0.5 mL) was added 1M NaOH (0.1 mL) and the mixture was stirred at room temperature overnight. The crude was purified with preparative HPLC on an ACE C8-column with a gradient of acetonitrile/0.1% TFA with UV-detection. The pure fractions were combined and the solvent was removed at reduced pressure to give 7.5 mg (76%) the target compound as a light brown oil.

HPLC 99%, R_{T}=1.55 min (System A. 10-97% MeCN), 100%, R_{T}=1.33 min (System B. 10-97% MeCN).

¹H NMR (400 MHz, MeOD) δ ppm 2.67-2.94 (m, 4H) 3.51-3.64 (m, 1H) 3.68-3.96 (m, 8H) 3.99 (s, 2H) 4.20-4.29 (m, 1H) 4.32-4.53 (m, 5H) 6.84 (dd, J=8.78, 2.26 Hz, 1H) 6.93-7.08 (m, 4H) 7.23-7.38 (m, 3H).

MS (ESI+) m/z 493 (M+H)⁺.

HRMS (EI) calcd for C₂₇32₉4₃O₅: 492.2373, found 492.2368.

### Example 128

### 3-(6-Methoxy-2,3,4,9-tetrahydro-1′H-spiro[beta-carboline-1,3′-pyrrolidin]-1′-yl)-1-phenylpropan-1-one trifluoroacetate

6-Methoxy-2,3,4,9-tetrahydrospiro[beta-carboline-1,3′-pyrrolidine] hydrochloride (COMPARATIVE EXAMPLE 1, 0.050 g, 0.194 mmol) was suspended in DMSO (0.15 mL) and DIPEA (4.6 eq, 0.203 mL) was added. 3-Chloro-1-phenylpropan-1-one (0.043 g, 0.253 mmol) dissolved in DMSO (0.30 mL) was added and a solution was formed. The reaction mixture was agitated at rt until no more product was formed according to LC-MS (18 h).

The reaction mixture was diluted with MeCN and purification was performed using preparative LC (System A, 15-45% MeCN over 5 min) affording 0.0053 g (7%) of a white solid.

HPLC 98% R_{T}=1.54 min (System A. 10-97% MeCN over 3 min), 98% R_{T=}1.36 min (System B. 10-97% MeCN over 3 min).

¹H NMR (270 MHz, CHLOROFORM-D) δ ppm 2.67 (d, J=5.94 Hz, 2H) 2.85-3.27 (m, 5H) 3.30-3.46 (m, 2H) 3.49-3.65 (m, 3H) 3.70 (s, 3H) 3.93-4.05 (m, 2H) 6.79 (d, J=2.23 Hz, 1H) 6.86 (dd, J=8.85, 2.41 Hz, 1H) 7.26-7.31 (m, 1H) 7.45 (t, J=7.55 Hz, 2H) 7.60 (t, J=7.36 Hz, 1H) 7.80-7.88 (m, 2H).

MS (ESI+) for C₂₄H₂₇N₃O₂ m/z 390 (M+H)⁺.

### Example 129

### 2-[2-(6-Methoxy-2,3,4,9-tetrahydro-1′H-spiro[beta-carboline-1,3′-pyrrolidin]-1′-yl)ethoxy]benzonitrile

The title compound was prepared from COMPARATIVE EXAMPLE 1 (66 mg, 0.26 mmol) and 2-(2-cyanophenoxy)ethyl methanesulfonate (56 mg) as described in the General Synthetic Procedure B, Method B to afford 0.0049 g.

HLPC 100%, Rt=1,398 min.

¹H-NMR (270 MHz, MeOH-d₆) δ 3.02-3.10 (m, 2H), 3.60-3.69 (m, 3H), 3.79-3.84 (s, 13H), 4.40-4.49 (m, 2H), 3.26-4.96 (m, 7H), 6.81-6.87 (m, 1H), 6.97-6.99 (m, 1H), 7.07-7.15 (m, 1H), 7.19-7.30 (m, 2H), 7.60-7.69 (m, 2H).

MS (ES) m/z 403 (M+H)⁺.

### Example 131

### N-Ethyl-6-methoxy-N,9-dimethyl-1′-(2-phenoxyethyl)-4,9-dihydrospiro[beta-carboline-1,3′-pyrrolidine]-2(3H)-carboxamide trifluoroacetate

Dry sodium hydride (1.8 mg, 0.074 mmol) was added to a stirred mixture of EXAMPLE 27 (0.033 mmol) in dry DMF (3 mL). Methyl iodide (6.6 μL, 0.074 mmol) was added after 10 min. The reaction mixture was stirred for 2 h before it was quenched with one drop of water. Evaporation using SpeedVac. The crude product ws purified by preparative HPLC (25-50%, 0.1% TFA). The fractions were pooled and evaporated affording 1.8 mg of the title compound.

HPLC 95% R_{T}=2.31 min (System A. 10-97% MeCN over 3 min), 95% R_{T=}2.14 min (System B. 10-90% MeCN over 3 min).

¹H NMR (400 MHz, CHLOROFORM-D) δ ppm 1.2 (m, 3H) 2.5 (m, 1H) 2.8 (m, 5H) 3.0 (m, 3H) 3.3 (m, 2H) 3.4 (s, 1H) 3.6 (s, 1H) 3.7 (m, 5H) 3.8 (m, 3H) 3.9 (m, 2H) 4.3 (m, 2H) 6.9 (m, 4H) 7.2 (m, 4H).

MS (ESI+) m/z 477 (M+H)⁺.

### Example 132

### 6-Methoxy-9-methyl-1′-(2-phenoxyethyl)-2,3,4,9-tetrahydrospiro[beta-carboline-1,3′-pyrrolidine] trifluoroacetate

COMPARATIVE EXAMPLE 101 (20.0 mg, 0.1 mmol), K₂CO₃ (20 mg, 0.1 mmol) and β-bromophenetole (15.6 mg, 0.1 mmol) were dissolved in CH₃CN (3 mL). The reaction mixture was allowed to stir at 70° C. overnight. The suspension was filtered and evaporated. The crude product was purified using preparative HPLC (19-28%, 0.1% TFA). SpeedVac of pooled fractions provided 13 mg (35%) of the title compound.

HPLC 99% R_{T}=1.80 min (System A. 10-97% MeCN over 3 min), 99% R_{T=}1.62 min (System B. 10-90% MeCN over 3 min).

¹H NMR (400 MHz, CHLOROFORM-D) δ ppm 2.60-3.07 (m, 5H) 3.23-3.49 (m, 3H) 3.55-4.00 (m, 9H) 4.00-4.44 (m, 4H) 6.70-7.00 (m, 3H) 7.00-7.36 (m, 5H).

MS (ESI+) m/z 392 (M+H)⁺.

### Example 134

### 1-(6-Methoxy-2,3,4,9-tetrahydro-1′H-spiro[beta-carboline-1,3′-pyrrolidin]-1′-yl)-3-phenylpropan-2-ol trifluoroacetate

6-Methoxy-2,3,4,9-tetrahydrospiro[beta-carboline-1,3′-pyrrolidine] hydrochloride (COMPARATIVE EXAMPLE 1, 0.020 g, 0.078 mmol) was suspended in DMSO (0.15 mL) and DIPEA (4.6 eq, 0.062 mL) was added. 3-(2-Bromoethyl)-1H-indole (0.023 g, 0.101 mmol) dissolved in DMSO (0.30 mL) was added. The reaction mixture was agitated at room temperature until no more product was formed according to LC-MS (13 days).

The reaction mixture was diluted with MeCN and purification was performed using preparative LC (System A, 10-40% MeCN over 5 min) affording 0.00135 g (4%) of yellow oil.

HPLC 100% R_{T}=1.49 min (System A. 10-97% MeCN over 3 min), 100% R_{T=}1.30 min (System B. 10-97% MeCN over 3 min).

¹H NMR (270 MHz, METHANOL-D3) δ ppm 2.62-2.79 (m, 2H) 2.83 (d, J=6.19 Hz, 2H) 3.05 (t, J=5.26 Hz, 2H) 3.12-3.28 (m, 3H) 3.49-3.77 (m, 4H) 3.80 (s, 3H) 4.08 (dd, J=12.74, 3.22 Hz, 1H) 4.22 (d, J=5.57 Hz, 1H) 6.85 (dd, J=8.91, 2.35 Hz, 1H) 6.98 (d, J=2.23 Hz, 1H) 7.15-7.39 (m, 6H).

MS (ESI+) for C₂₄H₂₉N₃O₂ m/z 392 (M+H)⁺.

### Example 135

### 6-Methoxy-2-(4-phenoxybutyl)-2,3,4,9-tetrahydrospiro[beta-carboline-1,3′-pyrrolidine]

The same procedure as for COMPARATIVE EXAMPLE 123 was used, but with (4-bromobutoxy)benzene (71 mg, 0.31 mmol) as electrophile. 59 mg (40% in two steps) of a yellow gum was obtained after deprotection.

HPLC 97%, R_{T}=1.91 (System C, MeCN 5-99% over 3 min).

¹H NMR (400 MHz, CD₃OD) δ ppm 1.83-1.95 (m, 2H) 1.99-2.12 (m, 2H) 2.73-3.14 (m, 4H) 3.19-3.28 (m, 2H) 3.71-3.89 (m, 5H) 3.81 (s, 3H) 4.03 (t, J=5.9 Hz, 2H) 4.23 (d, J=13.9 Hz, 1H) 6.79-6.91 (m, 4H) 6.96 (d, J=2.3 Hz, 1H) 7.17-7.24 (m, 2H) 7.30 (d, J=8.8 Hz, 1H).

MS (ESI+) for C₂₅H₃₁N₃O₂ m/z 406 (M+H)⁺.

### Example 136

### 1′-(2,3-Dihydro-1,4-benzodioxin-2-ylmethyl)-6-methoxy-2,3,4,9-tetrahydrospiro[beta-carboline-1,3′-pyrrolidine]

The title compound was prepared according to according to General Synthetic Procedure E, Method B using 100 μmol of EXAMPLE 10 at rt overnight. 30.9 mg (76%) of a clear oil was produced.

HPLC 97%, R_{T}=2.33 (System A, MeCN 5-60% over 3 min). HPLC 97%, R_{T}=2.12 (System B, MeCN 5-60% over 3 min).

¹H NMR (400 MHz, CD₃OD) δ ppm 2.55-2.77 (m, 2H) 3.03-3.09 (m, 2H) 3.33-3.61 (m, 4H) 3.64 (t, J=6.0 Hz, 2H) 3.68-3.77 (m, 1H) 3.81 (s, 3H) 3.84-3.99 (m, 1H) 4.04-4.11 (m, 1H) 4.34 (dd, J=11.5, 2.5 Hz, 1H) 4.55-4.63 (m, 1H) 6.78-6.91 (m, 5H) 6.96-7.00 (m, 1H) 7.27 (d, J=8.8 Hz, 1H).

MS (ESI+) for C₂₄H₂₇N₃O₃ m/z 406 (M+H)⁺.

### Example 137

### 6-Methoxy-1′-methyl-2-(2-phenoxyethyl)-2,3,4,9-tetrahydrospiro[beta-carboline-1,3′-pyrrolidine] trifluoroacetate

The same procedure as for EXAMPLE 45 was used, but with 6-methoxy-2-(2-phenoxyethyl)-2,3,4,9-tetrahydrospiro[beta-carboline-1,3′-pyrrolidine] (COMPARATIVE EXAMPLE 123). Purification with System A to afford 8.2 mg (23%) of a yellow oil.

HPLC 99%, R_{T}=2.36 (System B, MeCN 5-60% over 3 min). HPLC 98%, R_{T}=3.54 (System C, MeCN 5-60% over 3 min).

¹H NMR (400 MHz, CD₃OD) δ ppm 2.69-2.91 (m, 3H) 3.01 (s, 3H) 3.05-3.18 (m, 1H) 3.34-3.72 (m, 7H) 3.81 (s, 3H) 4.10-4.34 (m, 3H) 6.82 (dd, J=8.8, 2.4 Hz, 1H) 6.93-7.01 (m, 4H) 7.24-7.33 (m, 3H).

MS (ESI+) for C₂₄H₂₉N₃O₂ m/z 392 (M+H)⁺.

### Example 139

### Enantiomer (NB—The Chirality of the Compound is Relative)

### N-{2-[6-Methoxy-1′-(2-phenoxyethyl)-4,9-dihydrospiro[beta-carboline-1,3′-pyrrolidin]-2(3H)-yl]ethyl}urea

Conc. HCl (8 μL) and a solution of KOCN (7.7 mg, 0.095 mmol) in water (100 μL) were added to a suspension of EXAMPLE 168 (10.0 mg, 0.024 mmol) in water (100 μL). The reaction mixture was allowed to stir at 90° C. for 0.5 h. When no starting material was visible the reaction mixture was cooled on an ice bath. The solvent was then removed under reduced pressure. The crude product was dissolved in DMSO (100 μL) and MeOH (1200 μL) filtered and purified by direct injection to a preparative HPLC/MS System, eluted with MilliQ water, MeCN and NH₄HCO₃ 20-50% to give 2.3 mg, 21%, of a colorless oil.

HPLC 95%, R_{T}=1.655 min (System A. 10-97% MeCN), 95%, R_{T}=1.514 min (System B. 10-97% MeCN).

¹H NMR (400 MHz, MeOD) δ ppm 2.30-2.41 (m, 1H) 2.42-2.53 (m, 1H) 2.79-3.19 (m, 9H) 3.33-3.48 (m, 5H) 3.80 (s, 3H) 4.23 (t, J=5.3 Hz, 2H) 6.77 (m, 1H) 6.92-6.98 (m, 4H) 7.18 (d, J=8.7 Hz, 1H) 7.25-7.29 (m, 2H) MS (ESI+) m/z 464 (M+H)⁺.

### Example 140

### Enantiomer (NB—The Chirality of the Compound is Relative)

### N-Glycoloyl-6-methoxy-1′-(2-phenoxyethyl)-4,9-dihydrospiro[beta-carboline-1,3′-pyrrolidine]-2(3H)-carboxamide

Chloroacetyl isocyanate (6.7 μL, 0.1 mmol) and EXAMPLE 51 (30.0 mg, 0.1 mmol) were dissolved in 0.9 ml ACN and allowed to stir at room temperature for 15 minutes. The reaction mixture was used directly in the next step.

HPLC 92%, R_{T}=1.952 min (System A. 10-97% MeCN); 92%, R_{T}=1.810 min (System B. 10-97% MeCN).

MS (ESI+) m/z 497 (M+H)⁺.

To 300 μL of the reaction mixture (0.03 mmol), NaOH aq (1 mL, 2M) was added. The reaction mixture was allowed to stand at room temperature for two weeks. The solvent was then removed under reduced pressure, and the crude product was dissolved in MeOH and purified by direct injection to a preparative HPLC/MS System, eluated with MilliQ water, MeCN and MilliQ water, MeCN and NH₄HCO₃. The fractions containing the product were combined to give 1.1 mg, 9%.

HPLC 93%, R_{T}=1.786 min (System A. 10-97% MeCN); 91%, R_{T}=1.672 min (System B. 10-97% MeCN).

¹H NMR (400 MHz, MeOD) δ ppm 2.39-2.53 (m, 1H) 2.74-2.99 (m, 4H) 3.39-3.63 (m, 4H) 3.80 (s, 3H) 3.88-4.37 (m, 5H) 4.65 (s, 2H) 6.74 (d, J=8.2 Hz, 1H) 6.95-7.00 (m, 4H) 7.18 (d, J=8.7 Hz, 1H) 7.26-7.30 (m, 2H).

MS (ESI+) m/z 479 (M+H)⁺.

### Example 141

### Enantiomer (NB—The Chirality of the Compound is Relative)

### N′-{2-[6-Methoxy-1′-(2-phenoxyethyl)-4,9-dihydrospiro[beta-carboline-1,3′-pyrrolidin]-2(3H)-yl]ethyl}-N,N-dimethylurea

Synthesis was performed from EXAMPLE 168 and dimethylcarbamic chloride (2.7 μL) according to General Synthetic Procedure H, eluated with MilliQ water, MeCN and NH₄HCO₃ 10-40 to give 7.1 mg.

HPLC 92%, R_{T}=1.806 min (System A. 10-97% MeCN); 95%, R_{T}=1.653 min (System B. 10-97% MeCN).

¹H NMR (400 MHz, MeOD) δ ppm 2.02-2.14 (m, 1H) 2.24-2.29 (m, 1H) 2.64-2.83 (m, 4H) 2.85 (s, 6H) 2.87-3.16 (m, 9H) 3.27-3.34 (m, 1H) 3.79 (s, 3H) 4.16-4.19 (m, 2H) 6.67 (dd, J=8.7, 2.5 Hz, 1H) 6.88 (d, J=2.3 Hz, 1H) 6.91-6.98 (m, 3H) 7.12 (d, J=8.7 Hz, 1H) 7.24-7.28 (m, 2H)

MS (ESI+) m/z 492 (M+H)⁺.

### Example 142

### Enantiomer (NB—The Chirality of the Compound is Relative)

### 2-({2-[6-Methoxy-1′-(2-phenoxyethyl)-4,9-dihydrospiro[beta-carboline-1,3′-pyrrolidin]-2(3H)-yl]ethyl}amino)-N-methylacetamide

Synthesis was performed from EXAMPLE 168 and 2-chloro-N-methylacetamide (2.8 mg) according to General Synthetic Procedure H, eluated with MilliQ water, MeCN and NH₄HCO₃ 20-50 to give 2.0 mg, 15%.

HPLC 96%, R_{T}=1.596 min (System A. 10-97% MeCN), 95%, R_{T}=1.444 min (System B. 10-97% MeCN).

¹H NMR (400 MHz, MeOD) δ ppm 2.17-2.24 (m, 1H) 2.35-3.42 (m, 1H) 2.64-2.71 (m, 1H) 2.73 (s, 3H) 2.80-3.15 (m, 13H) 3.47 (s, 2H) 3.79 (s, 3H) 4.20 (t, J=5.3 Hz, 2H) 6.72 (dd, J=8.8, 2.5 Hz, 1H) 6.89 (d, J=2.3 Hz, 1H) 6.91-6-98 (m, 3H) 7.15 (d, J=8.7 Hz, 1H) 7.25-7.29 (m, 2H).

MS (ESI+) m/z 492 (M+H)⁺.

### Example 143

### Enantiomer (NB—The Chirality of the Compound is Relative)

### Methyl ({2-[6-methoxy-1′-(2-phenoxyethyl)-4,9-dihydrospiro[beta-carboline-1,3′-pyrrolidin]-2(3H)-yl]ethyl}amino)acetate

Synthesis was performed from EXAMPLE 168 and methyl chloroacetate (2.8 μL) according to General Synthetic Procedure H, eluated with MilliQ water, MeCN and NH₄HCO₃ 20-50 to give 8.6 mg, 74%.

HPLC 96%, R_{T}=1.641 min (System A. 10-97% MeCN); 95%, R_{T}=1.502 min (System B. 10-97% MeCN).

¹H NMR (400 MHz, MeOD) δ ppm 2.29-2.34 (m, 1H) 2.43-2.50 (m, 1H) 2.75-2.80 (m, 1H) 2.84-2.90 (m, 1H) 3.00-3.29 (m, 11H) 3.40 (d, J=10.8 Hz, 1H) 3.72 (d, J=1.9 Hz, 2H) 3.74 (s, 3H) 3.79 (s, 3H) 4.23 (t, J=5.2 Hz, 2H) 6.74 (dd, J=8.8, 2.5 Hz, 1H) 6.91 (d, J=2.3 Hz, 1H) 6.92-6.98 (m, 3H) 7.19 (d, J=8.8 Hz, 1H) 7.26-7.30 (m, 2H).

MS (ESI+) m/z 493 (M+H)⁺.

### Example 144

### Enantiomer (NB—The Chirality of the Compound is Relative)

### 2-Amino-N-{2-[6-methoxy-1′-(2-phenoxyethyl)-4,9-dihydrospiro[beta-carboline-1,3′-pyrrolidin]-2(3H)-yl]-2-oxoethyl}-2-methylpropanamide

Synthesis was performed from EXAMPLE 157 and N-(tert-butoxycarbonyl)-2-methylalanine (5.3 mg) according to General Synthetic Procedure G, eluated with NH₄HCO₃/ACN 33-63%, followed by boc-deprotection (25% TFA in DCM) to give 0.8 mg.

HPLC 100%, R_{T}=1.554 min (System A. 10-97% MeCN), 95%, R_{T}=1.419 min (System B. 10-97% MeCN).

¹H NMR (400 MHz, MeOD) δ ppm 1.24-1.37 (m, 2H) 1.52 (s, 6H) 2.26-2.34 (m, 1H) 2.71-2.85 (m, 3H) 3.12-3.20 (m, 3H) 3.40-3.76 (m, 3H) 3.79 (s, 3H) 3.84-3.91 (m, 1H) 4.22-4.24 (in, J=7.9 Hz, 3H) 6.72 (dd, J=8.8, 2.4 Hz, 1H) 7.0 (m, 4H) 7.2 (d, J=8.8 Hz, 1H) 7.3 (dd, J=8.8, 7.4 Hz, 2H).

MS (ESI+) m/z 520 (M+H)⁺.

### Example 145

### Enantiomer (NB—The Chirality of the Compound is Relative)

### 2-Methoxy-N-{2-[6-methoxy-1′-(2-phenoxyethyl)-4,9-dihydrospiro[beta-carboline-1,3′-pyrrolidin]-2(3H)-yl]-2-oxoethyl}acetamide

Synthesis was performed from EXAMPLE 157 and methoxyacetic acid (1.9 uL) according to General Synthetic Procedure G, eluated with NH₄HCO₃/ACN 36-66% (1.6 mg).

HPLC 100%, R_{T}=1.801 min (System A. 10-97% MeCN), 100%, R_{T}=1.688 min (System B. 10-97% MeCN).

¹H NMR (400 MHz, MeOD) δ ppm 2.28-2.39 (m, 1H) 2.74-2.84 (m, 3H) 3.11-3.25 (m, 2H) 3.35-3.44 (m, 2H) 3.46 (s, 3H) 3.47-3.48 (m, 1H) 3.64-3.74 (m, 2H) 3.80 (s, 3H) 3.82-3.87 (m, 1H) 3.96 (m, 2H) 4.23-4.24 (m, J=4.9 Hz, 2H) 4.26 (s, 2H) 6.72 (dd, J=8.7, 2.3 Hz, 1H 6.92-7.00 (m, 4H) 7.18 (d, J=8.8 Hz, 1H) 7.26-7.30 (m, 2H).

MS (ESI+) m/z 507 (M+H)⁺.

### Example 146

### Enantiomer (NB—The Chirality of the Compound is Relative)

### 2-Amino-N-{2-[6-methoxy-1′-(2-phenoxyethyl)-4,9-dihydrospiro[beta-carboline-1,3′-pyrrolidin]-2(3H)-yl]ethyl}-2-methylpropanamide

Synthesis was performed from EXAMPLE 168 and N-(tert-butoxycarbonyl)-2-methylalanine (5.3 mg) according to General Synthetic Procedure G, eluated with NH₄HCO₃/ACN 31-61%, followed by boc-deprotection to give 1.5 mg.

HPLC 100%, R_{T}=1.6046 min (System A. 10-97% MeCN), 100%, R_{T}=1.455 min (System B. 10-97% MeCN).

¹H NMR (400 MHz, MeOD) δ ppm 1.16-1.21 (m, 1H) 1.27-1.36 (m, 1H) 1.50 (s, 6H) 2.14-2.20 (m, 1H) 2.26-2.31 (m, 1H) 2.64-2.91 (m, 4H) 2.96-3.23 (m, 7H) 3.41 (t, J=6.6 Hz, 2H) 3.79 (s, 3H) 4.18-4.24 (m, 2H) 6.70 (dd, J=8.7, 2.4 Hz, 1H) 6.87 (d, J=2.3 Hz, 1H) 6.91-6.99 (m, 3H) 7.13 (d, J=8.8 Hz, 1H) 7.25-7.29 (m, 2H).

MS (ESI+) m/z 506 (M+H)⁺.

### Example 147

### Enantiomer (NB—The Chirality of the Compound is Relative)

### 2-Amino-N-{2-[6-methoxy-1′-(2-phenoxyethyl)-4,9-dihydrospiro[beta-carboline-1,3′-pyrrolidin]-2(3H)-yl]ethyl}acetamide

Synthesis was performed from EXAMPLE 168 and boc-glycine (4.4 mg) according to General Synthetic Procedure G, eluated with NH₄HCO₃/ACN 26-56%, followed by boc-deprotection (25% TFA in DCM) to give 0.8 mg.

HPLC 100%, R_{T}=1.583 min (System A. 10-97% MeCN), 100%, R_{T}=1.434 min (System B. 10-97% MeCN).

¹H NMR (400 MHz, MeOD) δ ppm 2.08-2.15 (m, 1H) 2.23-2.28 (m, 1H) 2.60-3.13 (m, 13H) 3.36-3.42 (m, 4H) 3.46-3.48 (m, 1H) 3.79 (s, 3H) 3.95-3.98 (m, 1H) 4.18 (t, J=5.2 Hz, 2H) 6.67 (dd, J=8.7, 2.4 Hz, 1H) 6.87-6.98 (m, 4H) 7.12 (d, J=8.7 Hz, 1H) 7.25-7.29 (m, J=8.0, 8.0 Hz, 2H)

MS (ESI+) m/z 478 (M+H)⁺.

### Example 148

### Enantiomer (NB—The Chirality of the Compound is Relative)

### 2-Methoxy-N-{2-[6-methoxy-1′-(2-phenoxyethyl)-4,9-dihydrospiro[beta-carboline-1,3′-pyrrolidin]-2(3H)-yl]ethyl}acetamide

Synthesis was performed from EXAMPLE 168 and methoxyacetic acid (1.9 μL) according to General Synthetic Procedure G, eluated with NH₄HCO₃/ACN 34-64% (1.4 mg).

HPLC 100%, R_{T}=1.829 min (System A. 10-97% MeCN); 100%, R_{T}=1.681 min (System B. 10-97% MeCN).

¹H NMR (400 MHz, MeOD) δ ppm 1.83-1.86 (m, 1H) 2.20-2.28 (m, 1H) 2.35-2.41 (m, 1H) 2.69-2.82 (m, 3H) 2.96-3.23 (m, 7H) 3.37 (s, 3H) 3.40-3.48 (m, 3H) 3.79 (s, 3H) 3.85 (s, 2H) 4.22 (t, J=4.3 Hz, 2H) 6.70 (dd, J=8.8, 2.1 Hz, 1H) 6.89 (d, J=2.3 Hz, 1H) 6.92-6.98 (m, 3H) 7.14 (d, J=8.7 Hz, 1H) 7.26-7.30 (m, 2H).

MS (ESI+) m/z 493 (M+H)⁺.

### Example 150

### Enantiomer (NB—The Chirality of the Compound is Relative)

### N-{2-[6-Methoxy-1′-(2-phenoxyethyl)-4,9-dihydrospiro[beta-carboline-1,3′-pyrrolidin]-2(3H)-yl]ethyl}morpholine-4-carboxamide trifluoroacetate

EXAMPLE 168 (10.0 mg, 0.024 mmol) and morpholine-4-carbonyl chloride (2.8 μL, 0.024 mmol) were dissolved in DCM (300 μL). TEA (3.3 μL, 0.024 mmol) was added to the solution and the reaction mixture was allowed to stir at room temperature for 30 min. The solvent was then removed under reduced pressure, to give a yellow oil. The oil was dissolved in MeOH and purified by direct injection to a preparative HPLC/MS System, eluated with MilliQ water, MeCN and MilliQ/MeCN/0.1% TFA. 10-40%. The fractions containing product were combined to give 6.3 mg, 54%.

HPLC 94%, R_{T}=1.777 min (System A. 10-97% MeCN); 95%, R_{T}=1.631 min (System B. 10-97% MeCN).

¹H NMR (400 MHz, MeOD) δ ppm 2.51-2.71 (m, 2H) 3.05 (t, J=5.6 Hz, 2H) 3.28-3.69 (m, 19H) 3.81 (s, 3H) 3.90 (d, J=12.5 Hz, 1H) 4.30 (t, J=5.0 Hz, 2H) 6.84 (dd, J=8.8, 2.4 Hz, 1H) 6.95-6.98 (m, 4H) 7.25-7.31 (m, 3H).

MS (ESI+) m/z 534 (M+H)⁺.

### Example 151

### Enantiomer (NB—The Chirality of the Compound is Relative)

### N-{2-[6-Methoxy-1′-(2-phenoxyethyl)-4,9-dihydrospiro[beta-carboline-1,3′-pyrrolidin]-2(3H)-yl]ethyl}acetamide trifluoroacetate

EXAMPLE 168 (10.0 mg, 0.024 mmol) was dissolved in DCM (300 μL). Acetic acid (1.4 mg, 0.024 mmol) and PyBOP (18.6 mg, 0.036 mmol) were added and allowed to stir shortly. DIPEA (10.3 μL, 0.06 mmol) was added and the reaction mixture was allowed to stir at room temperature for 1 h. The solvent was then removed under reduced pressure, to give a yellow oil. The oil was dissolved in MeOH and purifide by direct to a preparative HPLC/MS System, eluated with MilliQ water, MeCN and MilliQ/MeCN/0.1% TFA. 10-40%. The fractions containing product were combined to give 11.3 mg, 82%.

HPLC 96%, R_{T}=1.755 min (System A. 10-97% MeCN); 94%, R_{T}=1.607 min (System B. 10-97% MeCN).

¹H NMR (400 MHz, MeOD) δ ppm 1.92 (s, 3H) 2.59-2.75 (m, 2H) 2.85-3.17 (m, 4H) 3.48-3.61 (m, 8H) 3.74-3.78 (m, 1H) 3.80 (s, 3H) 4.01 (d, J=12.3 Hz, 1H) 4.34 (t, J=5.0 Hz, 2H) 6.80 (dd, J=8.8, 2.4 Hz, 1H) 6.95-6.99 (m, 4H) 7.23-7.31 (m, 3H).

MS (ESI+) m/z 463 (M+H)⁺.

### Example 152

### Enantiomer (NB—The Chirality of the Compound is Relative)

### 2-[6-Methoxy-1′-(2-phenoxyethyl)-4,9-dihydrospiro[beta-carboline-1,3′-pyrrolidin]-2(3H)-yl]acetamide

EXAMPLE 51 (1.0 g, 2.6 mmol), 2-bromoacetamide (0.37 g, 2.6 mmol) and K₂CO₃ (0.37 g, 2.6 mmol) were dissolved in DMF (30 mL) and heated at 100° C. for 1.5 h. When no starting material was left the reaction mixture was allowed to cool to room temperature. K₂CO₃ was filtered off and the solvent was removed under reduced pressure. The crude product was purified by flash chromatography by using initially chloroform 100% as eluent followed by chloroform/methanol 96/4. The solvent in the fractions containing the product was removed under reduced pressure to give 700 mg, 61%, yellow oil.

HPLC 96%, R_{T}=1.742 min (System A. 10-97% MeCN); 97%, R_{T}=1.616 min (System B. 10-97% MeCN).

¹H NMR (400 MHz, MeOD) δ ppm 2.14-2.30 (m, 2H) 2.65-3.05 (m, 12H) 3.79 (s, 3H) 4.16 (t, J=5.5 Hz, 2H) 6.68 (dd, J=8.7, 2.5 Hz, 1H) 6.88-6.98 (m, 4H) 7.15 (d, J=8.7 Hz, 1H) 7.24-7.28 (m, 2H).

MS (ESI+) m/z 435 (M+H)⁺.

### Example 153

### Enantiomer (NB—The Chirality of the Compound is Relative)

### 2-Amino-N-{2-[6-methoxy-1′-(2-phenoxyethyl)-4,9-dihydrospiro[beta-carboline-1,3′-pyrrolidin]-2(3H)-yl]-2-oxoethyl}acetamide trifluoroacetate

Synthesis was performed from EXAMPLE 157 and boc-glycine (4.4 mg) according to General Synthetic Procedure G, eluated with MilliQ water, MeCN and MilliQ/MeCN/0.1% TFA 10-40 and after boc-deprotection to give the title compound (3.8 mg).

HPLC 97%, R_{T}=1.536 min (System A. 10-97% MeCN), 96%, R_{T}=1.396 min (System B. 10-97% MeCN).

¹H NMR (400 MHz, MeOD) δ ppm 2.68-2.88 (m, 4H) 3.12-3.17 (m, 1H) 3.59-3.85 (m, 10H) 3.96-4.00 (m, 1H) 4.18-4.23 (m, 1H) 4.34-4.42 (m, 5H) 6.83 (dd, J=8.8, 2.4 Hz, 1H) 6.97-7.02 (m, 4H) 7.27-7.33 (m, 3H).

MS (ESI+) m/z 492 (M+H)⁺.

### Example 154

### Enantiomer (NB—The Chirality of the Compound is Relative)

### N-{2-[6-Methoxy-1′-(2-phenoxyethyl)-4,9-dihydrospiro[beta-carboline-1,3′-pyrrolidin]-2(3H)-yl]-2-oxoethyl}acetamide trifluoroacetate

EXAMPLE 157 (10.4 mg, 23.8 μmol) and acetyl chloride (1.7 μL, 23.8 μmol) were dissolved in DCM (300 μL). The reaction mixture was cooled on an ice bath and triethylamine (3.3 μL, 23.8 μmol) was added and allowed to stir at room temperature for 30 min. When the reaction was done the solvent was removed under reduced pressure, dissolved in MeOH and purified by direct injection to a preparative HPLC/MS System, eluated with MilliQ water, MeCN and MilliQ/MeCN/0.1% TFA 10-40%. The fractions containing product were combined to give 3.1 mg, 27%, of a yellow oil.

HPLC 98%, R_{T}=1.739 min (System A. 10-97% MeCN), 94%, R_{T}=1.608 min (System B. 10-97% MeCN).

¹H NMR (400 MHz, MeOD) δ ppm 2.01 (s, 3H) 2.65-2.72 (m, 1H) 2.79-2.88 (m, 3H) 3.54-3.86 (m, 8H) 3.96-4.01 (m, 1H) 4.18-4.23 (m, 3H) 4.33-4.39 (m, 3H) 6.82 (dd, J=8.8, 2.4 Hz, 1H) 6.97-7.03 (m, 4H) 7.26-7.32 (m, 3H).

MS (ESI+) m/z 492 (M+H)⁺.

### Example 155

### Enantiomer (NB—The Chirality of the Compound is Relative)

### 2-({2-[6-Methoxy-1′-(2-phenoxyethyl)-4,9-dihydrospiro[beta-carboline-1,3′-pyrrolidin]-2(3H)-yl]-2-oxoethyl}amino)ethanol trifluoroacetate

Synthesis was performed from EXAMPLE 157 and bromoethanol (1.8 uL) according to General Synthetic Procedure H, eluated with MilliQ water, MeCN and MilliQ/MeCN/0.1% TFA 5-20% to give the title compound (6.9 mg).

HPLC 98%, R_{T}=1.523 min (System A. 10-97% MeCN); 97%, R_{T}=1.380 min (System B. 10-97% MeCN).

¹H NMR (400 MHz, MeOD) δ ppm 2.65-2.89 (m, 4H) 3.20-3.31 (m, 1H) 3.50-3.61 (m, 2H) 3.70-3.95 (m, 10H) 4.18-4.49 (m, 6H) 6.83 (dd, J=8.8, 2.3 Hz, 1H) 6.97-7.05 (m, 4H) 7.30 (t, J=8.1 Hz, 3H) MS (ESI+) m/z 479 (M+H)⁺.

### Example 156

### Enantiomer (NB—The Chirality of the Compound is Relative)

### Methyl ({2-[6-methoxy-1′-(2-phenoxyethyl)-4,9-dihydrospiro[beta-carboline-1,3′-pyrrolidin]-2(3H)-yl]-2-oxoethyl}amino)acetate trifluoroacetate

Synthesis was performed from EXAMPLE 157 and methyl chloroacetate (2.8 mg) according to General Synthetic Procedure H, eluated with MilliQ water, MeCN and MilliQ/MeCN/0.1% TFA 10-40 to give the title compound (1.6 mg).

HPLC 97%, R_{T}=1.594 min (System A. 10-97% MeCN), 98%, R_{T}=1.447 min (System B. 10-97% MeCN).

¹H NMR (400 MHz, MeOD) δ ppm 2.73-2.91 (m, 4H) 3.46-3.59 (m, 2H) 3.71-3.94 (m, 10H) 4.00 (s, 2H) 4.18-4.47 (m, 6H) 6.85 (dd, J=8.8, 2.4 Hz, 1H) 6.97-7.03 (m, 4H) 7.28-7.33 (m, 3H).

MS (ESI+) m/z 507 (M+H)⁺.

### Example 157

### Enantiomer (NB—The Chirality of the Compound is Relative)

### {2-[6-Methoxy-1′-(2-phenoxyethyl)-4,9-dihydrospiro[beta-carboline-1,3′-pyrrolidin]-2(3H)-yl]-2-oxoethyl}amine trifluoroacetate

EXAMPLE 51 (0.57 g, 1.51 mmol), Boc-Glycine (0.40 g, 2.26 mmol) and PyBOP (1.18 g, 2.26 mmol) were dissolved in DCM (100 mL) and allowed. After 5 min DIPEA (660 μL, 3.77 mmol) was added to the reaction mixture and allowed to stir at room temperature over the weekend. When no progress of the conversion to product was noticed one more equivalent of Boc-Glycine, PyBOP and DIPEA was added and the reaction mixture was allowed to stir at rt overnight. The reaction was aborted after 70% conversion to product since no progress was observed.

The solvent was removed at reduced pressure. The remaining brown oil was chromatographed on a column of silica θ=45 mm L=110 mm initially with CHCl₃ as eluent followed by CHCl₃/MeOH 96/4. The fractions containing product was combined and the solvent was removed at reduced pressure and the remaining yellow oil was boc-deprotected by dissolving it in DCM and by adding TFA (25% TFA in DCM solution). The reaction mixture was allowed to stir overnight. The solvent was removed under reduced pressure and the remaining yellow oil was purified by direct injection on a preparativ HPLC System, MeCN:H₂O (0.1% TFA) 19-41%. The solvent from the fractions containing product was removed under reduced pressure to give a yellow oil, 442 mg, 55%.

HPLC 97%, R_{T}=1.486 min (System A. 10-97% MeCN); 100%, R_{T}=1.346 min (System B. 10-97% MeCN).

¹H NMR (400 MHz, MeOD) δ ppm 2.00-2.30 (m, 2H) 2.66-3.05 (m, 12H) 3.79 (s, 3H) 4.16 (t, J=5.5 Hz, 2H) 6.70 (dd, J=8.7, 2.5 Hz, 1H) 6.88-6.96 (m, 4H) 7.14 (d, J=8.7 Hz, 1H) 7.24-7.28 (m, J=8.8, 7.3 Hz, 2H).

MS (ESI+) m/z 435 (M+H)⁺.

### Example 158

### Enantiomer (NB—The Chirality of the Compound is Relative)

### 2-[6-Methoxy-1′-(2-phenoxyethyl)-4,9-dihydrospiro[beta-carboline-1,3′-pyrrolidin]-2(3H)-yl]ethanol

To a solution of methyl [6-methoxy-1′-(2-phenoxyethyl)-4,9-dihydrospiro[beta-carboline-1,3′-pyrrolidin]-2(3H)-yl]acetate (EXAMPLE 163, 18 mg, 40 μmol) in dry THF (3 mL) was added LAH (10 mg, 260 μmol) and the mixture was stirred at room temperature for 10 minutes. To the mixture was added EtOAc (0.2 mL) followed by MeOH (0.5 mL), the solvent was removed at reduced pressure and the remaining solid was trituated with MeOH (4×2 mL) and the solution was filtered through a small pad of silica and Celite, the solvent was again removed and the remain was dissolved in CHCl₃, filtered through a 0.45 μm filter and the solvent was evaporated at reduced pressure to give 15 mg (88%) of the target compound as a light brown oil.

HPLC 97%, R_{T}=1.69 min (System A. 10-97% MeCN), 94%, R_{T}=1.52 min (System B. 10-98% MeCN).

¹H NMR (400 MHz, CHLOROFORM-D) δ ppm 2.16-2.25 (m, 2H) 2.47-2.62 (m, 3H) 2.66 (d, J=8.53 Hz, 1H) 2.78-3.07 (m, 5H) 3.10-3.20 (m, 2H) 3.26-3.35 (m, 1H) 3.57-3.72 (m, 3H) 3.84 (s, 3H) 4.10-4.17 (m, 2H) 6.77 (dd, J=8.72, 2.45 Hz, 1H) 6.88-7.04 (m, 4H) 7.12 (d, J=8.78 Hz, 1H) 7.28-7.38 (m, 2H) 9.29 (s, 1H).

MS (ESI+) m/z 422 (M+H)⁺.

HRMS (EI) calcd for C₂₅H₁₆N₃O₃: 421.2370, found 421.2368.

### Example 159

### Enantiomer (NB—The Chirality of the Compound is Relative)

### 1′,2-Bis(2-hydroxyethyl)-6-methoxy-1′-(2-phenoxyethyl)-2,3,4,9-tetrahydrospiro[beta-carboline-1,3′-pyrrolidine] trifluoroacetate

To a solution of 6-methoxy-1′,2-bis(2-methoxy-2-oxoethyl)-1′-(2-phenoxyethyl)-2,3,4,9-tetrahydrospiro[beta-carboline-1,3′-pyrrolidine] bromide (EXAMPLE 162, 42 mg 70 μmol) in dry THF (5 mL) was added LAH (35 mg, 900 μmol) in small portions with a spatula. The mixture was stirred at room temperature for 10 minutes, one drop of water was added followed by conc HCl (0.1 mL) and the solvent was removed at reduced pressure. The residue was suspended in MeOH and the organic phase was purified on a preparative HPLC to give 17.9 mg (44%) of the target compound as a yellow oil.

HPLC 98%, R_{T}=1.84 min (System A. 10-97% MeCN), 93%, R_{T}=1.46 min (System B. 10-98% MeCN).

¹H NMR (400 MHz, MeOD) δ ppm 2.83-3.05 (m, 2H) 3.08-3.28 (m, 4H) 3.62-3.73 (m, 1H) 3.75-3.95 (m, 6H) 3.98-4.23 (m, 7H) 4.25-4.35 (m, J=7.78 Hz, 1H) 4.37-4.46 (m, J=12.55 Hz, 1H) 4.51 (t, J=3.89 Hz, 2H) 4.84-4.91 (m, 1H) 6.85 (dd, J=8.85, 2.45 Hz, 1H) 6.88-6.94 (m, 2H) 6.95-7.02 (m, 2H) 7.23-7.31 (m, 3H).

MS (ESI+) m/z 466 (M+H)⁺.

HRMS (EI) calcd for C₂₇H₃₆N₃O₄: 466.2710, found 466.2710.

### Example 162

### Enantiomer (NB—The Chirality of the Compound is Relative)

### 6-Methoxy-1′,2-bis(2-methoxy-2-oxoethyl)-1′-(2-phenoxyethyl)-2,3,4,9-tetrahydrospiro[beta-carboline-1,3′-pyrrolidine] bromide

The dialkylated product was purified from the same column with the same eluent as EXAMPLE 163.

HPLC 94%, R_{T}=2.14 min (System A. 10-97% MeCN), 93%, R_{T}=2.20 min (System B. 10-97% MeCN).

¹H NMR (400 MHz, CHLOROFORM-D) δ ppm 2.27 (s, 1H) 2.45 (s, 1H) 2.80-2.86 (m, 1H) 3.02-3.12 (m, 1H) 3.15-3.28 (m, 1H) 3.37-3.50 (m, 2H) 3.64-3.70 (m, 4H) 3.75 (s, 3H) 3.82 (s, 3H) 4.10-4.25 (m, 2H) 4.29-4.61 (m, 5H) 4.82 (dd, J=13.62, 4.83 Hz, 1H) 4.99-5.14 (m, 1H) 5.30-5.45 (m, 1H) 6.70-6.81 (m, 4H) 6.93 (t, J=7.40 Hz, 1H) 7.17-7.27 (m, 2H) 7.35 (d, J=8.53 Hz, 1H) 10.86 (s, 1H).

¹³C NMR (CDCl₃) δ 16.20, 32.65, 46.65, 49.83, 52.16, 52.81, 55.71, 62.42, 62.62, 62.69, 66.19, 66.30, 71.11, 99.84, 109.56, 112.35, 112.92, 114.14, 121.92, 126.38, 129.16, 129.60, 131.52, 153.73, 156.59, 165.93, 172.95.

MS (ESI+) m/z 522 (M+H)⁺.

### Example 163

### Enantiomer (NB—The Chirality of the Compound is Relative)

### Methyl [6-methoxy-1′-(2-phenoxyethyl)-4,9-dihydrospiro[beta-carboline-1,3′-pyrrolidin]-2(3H)-yl]acetate

K₂CO₃ (>300 mesh, 0.5 g 3.4 mmol) was added to a mixture of 6-methoxy-1′-(2-phenoxyethyl)-2,3,4,9-tetrahydrospiro[b-carboline-1,3′-pyrrolidine] (EXAMPLE 51, 0.64 g 1.7 mmol) and methyl bromoacetate (0.29 g 1.9 mmol) in dry DMF (6 mL). The reaction mixture was heated at 90° C. for 40 minutes. The reaction mixture was filtered, the solvent was removed at reduced pressure and the residue was chromatographed on a column of silica with CHCl₃/MeOH/conc aq NH₃ 95/5/0.2 as eluent to give 0.22 g (28%) of the target compound as an 1:1 adduct with DMF.

HPLC 99%, R_{T}=2.02 min (System A. 10-97% MeCN), 99%, R_{T}=2.56 min (System B. 10-97% MeCN).

¹H NMR (400 MHz, CHLOROFORM-D) δ ppm 2.15-2.25 (m, 1H) 2.26-2.36 (m, 1H) 2.49-2.60 (m, 2H) 2.64 (d, J=8.16 Hz, 1H) 2.87-3.03 (m, 3H) 3.08-3.19 (m, 2H) 3.20-3.33 (m, 3H) 3.49 (d, J=16.44 Hz, 1H) 3.73 (s, 3H) 3.82 (s, 3H) 4.12 (dd, J=5.33 Hz, 2H) 6.75 (dd, J=8.72, 2.45 Hz, 1H) 6.90 (d, J=2.38 Hz, 1H) 6.93-7.01 (m, 3H) 7.10 (d, J=8.78 Hz, 1H) 7.26-7.37 (m, 2H) 9.24 (s, 1H).

¹³C NMR (CDCl₃) δ 17.73, 37.57, 47.13, 51.46, 51.85, 52.67, 53.83, 55.88, 62.41, 64.55, 65.78, 100.24, 104.11, 110.83, 111.49, 114.41, 120.97, 127.11, 129.52, 130.37, 140.65, 153.64, 158.54, 162.39, 172.03.

MS (ESI+) m/z 450 (M+H)⁺.

HRMS (EI) calcd for C₂₆H₃₁N₃O₄: 449.2315, found 449.2332.

### Example 165

### Enantiomer (NB—The Chirality of the Compound is Relative)

### 1′-[(1S)-2-(4-Fluorophenoxy)-1-methylethyl]-6-methoxy-2,3,4,9-tetrahydrospiro[beta-carboline-1,3′-pyrrolidine] trifluoroacetate

(2R)-1-(4-Fluorophenoxy)propan-2-ol (0.056 g, 0.329 mmol), methanesulfonyl chloride (1.2 eq, 0.031 mL) and triethylamine (2 eq, 0.092 mL) in DCM (1 mL) were agitated at rt for 2 h. 1M HCl was added and the layers were separated. The organic phase was concentrated in vacuum and the crude product was used in the next step without further purification.

6-Methoxy-2,3,4,9-tetrahydrospiro[beta-carboline-1,3′-pyrrolidine] hydrochloride (COMPARATIVE EXAMPLE 182, 0.070 g, 0.272 mmol) was dissolved in DMSO (0.300 mL) and DIPEA (3.5 eq, 0.166 mL) was added. The (1R)-2-(4-fluorophenoxy)-1-methylethyl methanesulfonate from above (0.081 g, 0.326 mmol) dissolved in DMSO (0.150 mL) was added. The reaction mixture was agitated at room temperature until no more product was formed according to LC-MS (20 days).

The reaction mixture was diluted with MeCN and purification was performed using preparative LC (System A, 15-45% MeCN over 5 min) affording 0.0037 g (3%) of yellow gum.

HPLC 95% R_{T}=1.78 min (System A. 10-97% MeCN over 3 min), 95% R_{T}=1.54 min (System B. 10-97% MeCN over 3 min).

¹H NMR (270 MHz, CHLOROFORM-D) δ ppm 1.42 (d, J=6.68 Hz, 3H) 2.39-2.57 (m, 2H) 2.85-3.00 (m, 2H) 3.22-3.72 (m, 6H) 3.80 (s, 3H) 3.82 (d, J=4.70 Hz, 1H) 4.06 (dd, J=10.14, 6.06 Hz, 2H) 6.71-7.00 (m, 6H) 7.28 (d, J=8.78 Hz, 1H).

MS (ESI+) for C₂₄H₂₈FN₃O₂ m/z 410 (M+H)⁺.

### Example 166

### Enantiomer (NB—The Chirality of the Compound is Relative)

### 6-Methoxy-1′-(1-methyl-2-phenoxyethyl)-2,3,4,9-tetrahydrospiro[beta-carboline-1,3′-pyrrolidine] trifluoroacetate

1-Phenoxypropan-2-ol (0.050 g, 0.329 mmol), methanesulfonyl chloride (1.2 eq, 0.031 mL) and triethylamine (2 eq, 0.092 mL) in DCM (1 mL) was agitated at rt for 2 h. HCl (1M) was added and the layers were separated. The organic phase was concentrated in vacuum and the crude product was used in the next step without further purification.

6-Methoxy-2,3,4,9-tetrahydrospiro[beta-carboline-1,3′-pyrrolidine] hydrochloride (COMPARATIVE EXAMPLE 182, 0.070 g, 0.272 mmol) was dissolved in DMSO (0.300 mL) and DIPEA (3.5 eq, 0.166 mL) was added. The 1-methyl-2-phenoxyethyl methanesulfonate from above (0.075 g, 0.326 mmol) dissolved in DMSO (0.150 mL) was added. The reaction mixture was agitated at room temperature until no more product was formed according to LC-MS (20 days).

The reaction mixture was diluted with MeCN and purification was performed using preparative LC (System A, 15-45% MeCN over 5 min) affording 0.0046 g (4%) of yellow gum.

HPLC 90% R_{T}=1.75 min (System A. 10-97% MeCN over 3 min), 90% R_{T}=1.51 min (System B. 10-97% MeCN over 3 min).

¹H NMR (270 MHz, CHLOROFORM-D) δ ppm 1.18-1.47 (m, 3H) 2.38-2.69 (m, 4H) 2.93 (d, J=5.81 Hz, 2H) 3.23-3.52 (m, 4H) 3.79 (s, 3H) 3.81 (d, J=1.86 Hz, 1H) 3.95-4.16 (m, 2H) 6.78-6.90 (m, 5H) 6.99 (t, J=7.05 Hz, 1H) 7.08-7.16 (m, 1H) 7.29 (s, 1H).

MS (ESI+) for C₂₄H₂₉N₃O₂ m/z 392 (M+H)⁺.

### Example 168

### Enantiomer (NB—The Chirality of the Compound is Relative)

### 2-[6-Methoxy-1′-(2-phenoxyethyl)-4,9-dihydrospiro[beta-carboline-1,3′-pyrrolidin]-2(3H)-yl]ethanamine

EXAMPLE 152 was dissolved in THF (20 ml) and LiAlH₄ was added slowly (5.5 mL, 1M in THF) to the solution. The reaction mixture was refluxed at 70° C. for 3 h. The reaction was cooled on an ice bath and water (0.25 mL) was added dropwise to quench the reaction. After 10 min of stirring 2M NaOH (0.25 mL) was added and after a further 10 minutes 0.75 mL of water was added. The crystals formed were filtered off and the solvent was evaporated, to give 206 mg, 67%, of a yellow oil.

HPLC 92%, R_{T}=1.437 min (System A. 10-97% MeCN), 91%, R_{T}=1.575 min (System B. 10-97% MeCN).

¹H NMR (400 MHz, MeOD) δ ppm 2.08-2.15 (m, 1H) 2.21-2.33 (m, 1H) 2.59-3.18 (m, 14H) 3.78 (s, 3H) 4.18 (t, J=5.5 Hz, 2H) 6.67 (dd, J=8.7, 2.5 Hz, 1H) 6.87 (d, J=2.4 Hz, 1H) 6.92 (t, J=7.3 Hz, 1H) 6.98 (m, 2H) 7.11 (d, J=8.7 Hz, 1H) 7.25-7.29 (m, 2H).

MS (ESI+) m/z 421 (M+H)⁺.

### Example 169

### [6-Methoxy-1′-(2-phenoxyethyl)-4,9-dihydrospiro[beta-carboline-1,3′-pyrrolidin]-2(3H)-yl]acetic acid acetate

HPLC 99%, R_{T}=1.79 min (System A. 10-97% MeCN), 97%, R_{T}=1.63 min (System B. 10-98% MeCN).

¹H NMR (400 MHz, MeOD) δ ppm 1.91-1.95 (m, 3H) 2.35-2.46 (m, 1H) 2.54-2.65 (m, 1H) 2.83-3.02 (m, 2H) 3.16-3.27 (m, 3H) 3.48-3.56 (m, 3H) 3.59-3.70 (m, 2H) 3.76-3.84 (m, 4H) 4.27 (t, J=5.33 Hz, 2H) 6.78 (dd, J=8.78, 2.51 Hz, 1H) 6.91-7.01 (m, 4H) 7.19-7.31 (m, 3H).

MS (ESI+) m/z 436 (M+H)⁺.

### Example 171

### Enantiomer (NB—The Chirality of the Compound is Relative)

### 3-Hydroxy-4-({2-[(1S)-6-methoxy-1′-(2-phenoxyethyl)-4,9-dihydrospiro[beta-carboline-1,3′-pyrrolidin]-2(3H)-yl]ethyl}amino)cyclobut-3-ene-1,2-dione trifluoroacetate

3,4-Diethoxycyclobut-3-ene-1,2-dione (14 μL, 0.1 mmol), NaOH (3.9 mg, 0.1 mmol), 2-[6-methoxy-1′-(2-phenoxyethyl)-4,9-dihydrospiro[beta-carboline-1,3′-pyrrolidin]-2(3H)-yl]ethanamine (EXAMPLE 168, 41 mg, 0.1 mmol) and methanol (1 mL) were stirred at rt for 2 h. The solvent was removed and THF (1 mL), water (1 mL) and 2M HCl (1 mL) were added and the reaction was stirred for 16 h at rt. Purification of the product was done by preparative HPLC using acetonitrile-water gradients containing 0.1% triflouroacetic acid to yield 5.2 mg (10%).

HPLC 100%, R_{T}: 1.709 (10-97% MeCN over 3 min).

¹H NMR (500 MHz, DMSO-d₆) δ ppm 1.53-2.39 (m, 8H) 2.52-2.69 (m, 4H) 2.81-3.18 (m, 4H) 2.99 (s, 3H) 3.56 (s, 2H) 5.97 (dd, J=8.79, 2.51 Hz, 1H) 6.12-6.18 (m, 4H) 6.40 (d, J=8.79 Hz, 1H) 6.47 (t, J=8.01 Hz, 2H).

L MS (ESI+) m/z 517 (M+H)⁺.

### Example 172

### Enantiomer (NB—The Chirality of the Compound is Relative)

### 1-[6-Methoxy-2,3,4,9-tetrahydro-1′H-spiro[beta-carboline-1,3′-pyrrolidin]-1′-yl]-3-phenoxypropan-2-ol trifluoroacetate

6-Methoxy-2,3,4,9-tetrahydrospiro[beta-carboline-1,3′-pyrrolidine] hydrochloride (COMPARATIVE EXAMPLE 182, 0.050 g, 0.194 mmol) and 2-(phenoxymethyl)oxirane (0.044 g, 0.291 mmol) in MeOH (1 mL) was heated with stirring to 60° C. for 16 h.

Purification performed using preparative LC (System A, 15-45% MeCN over 5 min) afforded 0.0106 g (13%) of yellow gum.

HPLC 99% R_{T}=1.61 min (System A. 10-97% MeCN over 3 min), 99% R_{T}=1.40 min (System B. 10-97% MeCN over 3 min).

¹H NMR (270 MHz, METHANOL-D3) δ ppm 2.55-2.85 (m, 2H) 3.06 (t, J=5.94 Hz, 2H) 3.34-3.39 (m, 1H) 3.49-3.77 (m, 5H) 3.81 (s, 3H) 3.90-4.01 (m, 2H) 4.01-4.06 (m, 2H) 4.32 (dd, J=7.67, 4.95 Hz, 1H) 6.80-6.89 (m, 1H) 6.89-7.01 (m, 4H) 7.20-7.32 (m, 3H).

MS (ESI+) for C₂₄H₂₉N₃O₃ m/z 408 (M+H)⁺.

### Example 174

### Enantiomer (NB—The Chirality of the Compound is Relative)

### 6-Methoxy-1′-[(2E)-3-phenylprop-2-en-1-yl]-2,3,4,9-tetrahydrospiro[beta-carboline-1,3′-pyrrolidine] trifluoroacetate

6-Methoxy-2,3,4,9-tetrahydrospiro[beta-carboline-1,3′-pyrrolidine] hydrochloride (COMPARATIVE EXAMPLE 182, 0.050 g, 0.194 mmol) was dissolved in DMSO (0.150 mL) and DIPEA (4.6 eq, 0.156 mL) was added. Cinnamyl bromide (0.050 g, 0.253 mmol) dissolved in DMSO (0.300 mL) was added.

The reaction mixture was agitated at room temperature until no more product was formed according to LC-MS (3 days). Purification was performed using preparative LC (System A, 20-50% MeCN over 5 min) affording 0.0028 g (4%) of a white solid.

HPLC 98% R_{T}=1.68 min (System A. 10-97% MeCN over 3 min), 98% R_{T}=1.52 min (System B. 10-97% MeCN over 3 min).

¹H NMR (500 MHz, CHLOROFORM-D) δ ppm 1.80-2.77 (m, 5H) 3.05 (s, 3H) 3.48 (s, 6H) 3.94 (s, 2H) 5.93-6.33 (m, 1H) 6.75 (d, J=16.33 Hz, 1H) 6.82 (s, 1H) 6.88 (dd, J=8.95, 2.35 Hz, 1H) 7.28-7.36 (m, 4H) 7.37-7.40 (m, 2H).

MS (ESI+) for C₂₄H₂₇N₃₀ m/z 374 (M+H)⁺.

### Example 175

### Enantiomer (NB—The Chirality of the Compound is Relative)

### 6-Methoxy-1′-(3-phenylpropyl)-2,3,4,9-tetrahydrospiro[beta-carboline-1,3′-pyrrolidine] trifluoroacetate

6-Methoxy-2,3,4,9-tetrahydrospiro[beta-carboline-1,3′-pyrrolidine] hydrochloride (COMPARATIVE EXAMPLE 182, 0.050 g, 0.194 mmol) was dissolved in DMSO (0.150 mL) and DIPEA (4.6 eq, 0.156 mL) was added. 3-Bromopropylbenzene (0.050 g, 0.253 mmol) dissolved in DMSO (0.300 mL) was added.

The reaction mixture was agitated at room temperature until no more product was formed according to LC-MS (3 days). Purification was performed using preparative LC (System A, 20-50% MeCN over 5 min) affording 0.0130 g (18%) of a yellow gum.

HPLC 98% R_{T}=1.64 min (System A. 10-97% MeCN over 3 min), 100% R_{T}=1.51 min (System B. 10-97% MeCN over 3 min).

¹H NMR (500 MHz, CHLOROFORM-D) δ ppm 1.79 (s, 1H) 2.02 (s, 1H) 2.36-2.73 (m, 4H) 2.89 (s, 2H) 3.02 (s, 2H) 3.11 (s, 1H) 3.25 (s, 1H) 3.47 (s, 2H) 3.66 (s, 1H) 3.81 (s, 3H) 3.95 (s, 1H) 6.85 (s, 1H) 6.90 (dd, J=8.79, 2.20 Hz, 1H) 7.12 (d, J=7.22 Hz, 2H) 7.22 (t, J=7.38 Hz, 1H) 7.26-7.32 (m, 3H).

MS (ESI+) for C₂₄H₂₉N₃₀ m/z 376 (M+H)⁺.

### Example 176

### Enantiomer (NB—The Chirality of the Compound is Relative)

### N-{2-[6-Methoxy-1′-(2-phenoxyethyl)-4,9-dihydrospiro[beta-carboline-1,3′-pyrrolidin]-2(3H)-yl]ethyl}-2-furamide

EXAMPLE 168 (10.0 mg, 0.024 mmol), 2-furoyl chloride (2.3 μL, 0.024 mmol) and K₂CO₃ (6.6 mg, 0.048 mmol) were suspended in ACN (300 μL) and allowed to stir at 50° C. for 1 h. The reaction mixture was filtered and diluted with MeOH and purified by direct injection to a preparativ HPLC/UV System, MeCN:H₂O (5 mM ammonium acetate) 45-71% giving 3.4 mg (8%) of yellow gum.

HPLC 100%, R_{T}=1.781 min (System A. 10-97% MeCN), 100%, R_{T}=1.963 min (System B. 10-97% MeCN).

¹H NMR (400 MHz, MeOD) δ ppm 2.08-2.15 (m, 1H) 2.25-2.31 (m, 1H) 2.62-2.68 (m, 1H) 2.78-2.85 (m, 3H) 2.89-3.16 (m, 8H) 3.45-3.57 (m, 2H) 3.79 (s, 3H) 4.10-4.18 (m, 2H) 6.54 (dd, J=3.5, 1.8 Hz, 1H) 6.67 (dd, J=8.7, 2.5 Hz, 1H) 6.88 (d, J=2.1 Hz, 1H) 6.90-6.95 (m, 3H) 7.07 (dd, J=3.5, 0.8 Hz, 1H) 7.12 (d, J=8.7 Hz, 1H) 7.22-7.26 (m, 2H) 7.60 (dd, J=1.7, 0.8 Hz, 1H).

MS (ESI+) m/z 515 (M+H)⁺.

### Example 177

### Enantiomer (NB—The Chirality of the Compound is Relative)

### N-{2-[6-Methoxy-1′-(2-phenoxyethyl)-4,9-dihydrospiro[beta-carboline-1,3′-pyrrolidin]-2(3H)-yl]ethyl}isoxazole-5-carboxamide trifluoroacetate

EXAMPLE 168 (10.0 mg, 0.024 mmol), isoxazole-5-carbonyl chloride (3.1 μL, 0.024 mmol) and K₂CO₃ (6.6 mg, 0.048 mmol) were suspended in ACN (300 μL) and allowed to stir at 50° C. for 1 h. The reaction mixture was filtered and diluted with MeOH and purified by direct injection to a preparativ HPLC/UV System, MeCN:H₂O (0.1% TFA) 30-53% giving 11.9 mg (79%) of yellow gum.

HPLC 94%, R_{T}=1.908 min (System A. 10-97% MeCN), 94%, R_{T}=1.744 min (System B. 10-97% MeCN).

¹H NMR (400 MHz, MeOD) δ ppm 2.62-2.83 (m, 3H) 2.98-3.27 (m, 3H) 3.47-3.75 (m, 8H) 3.80 (s, 3H) 3.82-3.95 (m, 1H) 4.07 (d, J=12.5 Hz, 1H) 4.34 (t, J=5.0 Hz, 2H) 6.80 (dd, J=8.8, 2.4 Hz, 1H) 6.92 (d, J=1.9 Hz, 1H) 6.94-6.98 (m, 4H) 7.23-7.29 (m, 3H) 8.48 (d, J=1.9 Hz, 1H).

MS (ESI+) m/z 516 (M+H)⁺.

### Example 178

### Enantiomer (NB—The Chirality of the Compound is Relative)

### 2-Hydroxy-N-{2-[6-methoxy-1′-(2-phenoxyethyl)-4,9-dihydrospiro[beta-carboline-1,3′-pyrrolidin]-2(3H)-yl]ethyl}acetamide trifluoroacetate

EXAMPLE 168 (30.0 mg, 0.071 mmol), (benzyloxy)acetyl chloride (11.3 μL, 0.071 mmol) and K₂CO₃ (19.8 mg, 0.144 mmol) were suspended in ACN (1 mL) and allowed to stir at 50° C. overnight. The reaction mixture was filtered and diluted with MeOH and purified by direct injection to a preparative HPLC/UV System, MeCN:H₂O (0.1% TFA) 23-45% giving 3.4 mg (8%) of yellow gum.

HPLC 98%, R_{T}=1.699 min (System A. 10-97% MeCN), 98%, R_{T}=1.524 min (System B. 10-97% MeCN).

¹H NMR (400 MHz, MeOD) δ ppm 2.58-3.24 (m, 5H) 3.47-3.65 (m, 9H) 3.81-3.85 (m, 2H) 3.80 (s, 3H) 3.98 (s, 2H) 4.00-4.05 (m, 1H) 4.36 (t, J=5.1 Hz, 2H) 6.80 (dd, J=8.8, 2.4 Hz, 1H) 6.95 (d, J=2.3 Hz, 1H) 6.96-7.00 (m, 3H) 7.24 (d, J=8.8 Hz, 1H) 7.26-7.31 (m, 2H).

MS (ESI+) m/z 479 (M+H)⁺.

### Example 179

### Enantiomer (NB—The Chirality of the Compound is Relative)

### N-{2-[6-methoxy-1′-(2-phenoxyethyl)-4,9-dihydrospiro[beta-carboline-1,3′-pyrrolidin]-2(3H)-yl]ethyl}morpholine-2-carboxamide trifluoroacetate

EXAMPLE 168 (30.0 mg, 0.071 mmol) was dissolved in DCM (300 μL). 4-Benzylmorpholine-2-carboxylic acid hydrochloride (18.4 mg, 0.071 mmol) and PyBOP (55.6 mg, 0.11 mmol) were added and allowed to stir shortly. DIPEA (31 μL, 0.18 mmol) was added and the reaction mixture was allowed to stir at room temperature for 1 h. The solvent was then removed under reduced pressure, to give a yellow oil. The oil was dissolved in MeOH and purified by direct injection to a preparative HPLC/UV System, MeCN:H₂O (5 mM ammonium acetate) 56-86% giving 22.4 mg (50%) of a white solid. No further characterization, the solid was used directly in the next step.

The benzyl amine from above (22.4 mg, 0.036 mmol), ammonium formate (3.4 mg, 0.054 mmol) and 10% Pd/C (2.0 mg, 0.002 mmol) were dissolved in MeOH and stirred at 140° C. for 180 s in a microwave oven. The reaction mixture was filtered and purified by direct injection to a preparative HPLC/UV System, MeCN:H₂O (0.1% TFA) 20-42%. The fractions containing product were combined to give a yellow oil. 13.2 mg, 69%.

HPLC 99%, R_{T}=1.566 min (System A. 10-97% MeCN), 99%, R_{T}=1.397 min (System B. 10-97% MeCN).

¹H NMR (400 MHz, MeOD) δ ppm 2.62-2.90 (m, 3H) 2.96-3.21 (m, 6H) 3.46-3.073 (m, 9H) 3.8 (s, 3H) 3.83-3.92 (m, 2H) 4.10-4.18 (m, 2H) 4.28-4.32 (dt, J=11.3, 3.3, 3.0 Hz, 1H) 4.37 (t, J=4.9 Hz, 2H) 6.81 (dd, J=2.4 Hz, 8.8 Hz, 1H) 6.94 (d, J=2.4 Hz, 1H) 6.97-7.00 (m, 3H) 7.25 (d, J=8.8 Hz, 1H) 7.27-7.32 (m, 2H).

MS (ESI+) m/z 534 (M+H)⁺.

### Example 180

### Enantiomer (NB—The Chirality of the Compound is Relative)

### 2-(Dimethylamino)-N-{2-[6-methoxy-1′-(2-phenoxyethyl)-4,9-dihydrospiro[beta-carboline-1,3′-pyrrolidin]-2(3H)-yl]ethyl}acetamide trifluoroacetate

EXAMPLE 168 (10.0 mg, 0.024 mmol) was dissolved in DCM (300 μL). N,N-dimethylglycine (2.5 mg, 0.024 mmol) and PyBOP (18.6 mg, 0.036 mmol) were added and allowed to stir shortly. DIPEA (10.3 μL, 0.059 mmol) was added and the reaction mixture was allowed to stir at room temperature for 1 h. The solvent was then removed under reduced pressure, to give a yellow oil. The oil was dissolved in MeOH and purified by direct injection to a preparative HPLC/UV System, MeCN:H₂O (0.1% TFA) 21-43% giving 10.6 mg (48%) of yellow gum.

HPLC 97%, R_{T}=1.593 min (System A. 10-97% MeCN), 97%, R_{T}=1.419 min (System B. 10-97% MeCN).

¹H NMR (400 MHz, MeOD) δ ppm 2.65-2.86 (m, 3H) 2.89 (s, 6H) 2.99-3.20 (m, 3H) 3.48-3.73 (m, 8H) 3.80 (s, 3H) 3.90-3.91 (m, 1H) 3.94 (s, 2H) 4.18 (d, J=12.9 Hz, 1H) 4.38 (t, J=5.1 Hz, 2H) 6.80 (dd, J=8.8, 2.4 Hz, 1H) 6.94 (d, J=2.3 Hz, 1H) 6.98-7.00 (m, 3H) 7.24-7.32 (m, 3H).

MS (ESI+) m/z 506 (M+H)⁺.

### Example 181

### 2-[6-Methoxy-1′-(2-phenoxyethyl)-4,9-dihydrospiro[beta-carboline-1,3′-pyrrolidin]-2(3H)-yl]ethanamine

LiAlH₄ (1.05 g, 27.6 mmol) was added to 2-[6-methoxy-1′-(2-phenoxyethyl)-4,9-dihydrospiro[beta-carboline-1,3′-pyrrolidin]-2(3H)-yl]acetamide (EXAMPLE 38, 1.5 g, 3.5 mmol) in dry THF (50 mL) and the reaction was refluxed for 16 h, let to rt and than cooled with an ice-bath. Na₂SO₄.10H₂O (60 g) was added portionwise to the stirred solution. After the addition the mixture was stirred for 1 h, filtered through celite and the solvent was removed to afford 1.1 g (75%).

HPLC 67%, R_{T}: 1.666 (10-97% MeCN over 3 min).

¹H NMR (270 MHz, Methanol-d₃) δ ppm 1.58-1.61 (m, 1H) 1.84-1.89 (m, 1H) 2.50-3.10 (m, 8H) 3.54-3.57 (m, 2H) 3.69-3.72 (m, 6H) 3.78 (s, 3H) 4.18 (t, J=5.57 Hz, 2H) 6.69-6.72 (m, 1H) 6.87-6.99 (m, 4H) 7.11-7.19 (m, 1H) 7.20-7.29 (m, 2H).

MS (ESI+) m/z 421 (M+H)⁺.

### Comparative Example 182

### Enantiomer (NB—The Chirality of the Compound is Relative)

### 6-Methoxy-2,3,4,9-tetrahydrospiro[beta-carboline-1,3′-pyrrolidine]

The free base of 1′-benzyl-6-methoxy-2,3,4,9-tetrahydrospiro[beta-carboline-1,3′-pyrrolidine] (second enantiomer from resolvation experiment in EXAMPLE 126) was extracted from alkaline water (8M NaOH)/CHCl₃. The organic phase was dried with MgSO₄ and the solvent was removed under reduced pressure. The remaining light yellow oil (2.5 g, 8.6 mmol) was dissolved in MeOH (100 mL) and put in a three-necked roundflask. Palladium hydroxide on carbon 20%, with 60% moisture (226 mg, 0.11 mmol) was added, and the reaction mixture was hydrogenated at atmospheric pressure (H₂-baloon) and allowed to stir at 50° C. overnight. The reaction mixture was then allowed to cool to room temperature, and was filtered through celite. Solvent removed under reduced pressure, afforded a yellow oil, 1.9 g (100%). The crude was used in the next synthetic step without further purification.

HPLC 89%, R_{T}=1.071 min (System A. 10-97% MeCN), 87%, R_{T}=0.929 min (System B. 10-97% MeCN).

MS (ESI+) m/z 258 (M+H)⁺.

alt.

The title compound was provided from COMPARATIVE EXAMPLE 1 using chiral preparative HPLC.

Chiral HPLC 100%, R_{T}=14.6 min.

### Comparative Example 183

### 1-(2-Phenoxyethyl)pyrrolidin-3-one

β-Bromophenetole (5.00 g, 24.9 mmol) and pyrrolidin-3-ol (2.17 g, 24.9 mmol) were dissolved in MeCN (20 mL) and K₂CO₃ (3.43 g, 24.9 mmol) was added. The reaction mixture was heated with stirring to 40° C. for 16 h. The reaction mixture was filtered and concentrated in vacuum, affording 5.18 g of crude product as orange oil, which was used in the next step without further purification.

HPLC 90% R_{T}=1.55 min (System A. 5-60% MeCN over 3 min), 88% R_{T}=1.28 min (System B. 5-60% MeCN over 3 min).

¹H NMR (270 MHz, CHLOROFORM-D) δ ppm 1.66-1.84 (m, 1H) 2.10-2.28 (m, 1H) 2.32-2.48 (m, 2H) 2.55-2.70 (m, 1H) 2.74-2.84 (m, 1H) 2.86-2.92 (m, 2H) 2.93-3.06 (m, 1H) 3.99-4.16 (m, 2H) 4.28-4.42 (m, 1H) 6.83-7.01 (m, 3H) 7.18-7.38 (m, 2H).

MS (ESI+) for C₁₂H₁₇NO₂ m/z 208 (M+H)⁺.

To a stirred solution of oxalyl chloride (4.6 g, 36.5 mmol) in CH₂Cl₂ (80 mL), DMSO (5.2 mL, 73.1 mmol) dissolved in CH₂Cl₂ (15 mL) was added at −67° C. After 10 minutes of stirring 1-(2-phenoxyethyl)pyrrolidin-3-ol (5.3 g, 25.4 mmol) dissolved in CH₂Cl₂ (40 mL) was added and after a further 15 min Et₃N (14.6 g, 143.9 mmol) was added and the reaction mixture was brought up from the cold and allowed to reach room temperature. Saturated NaHCO₃ (aq) was added 45 min later and the mixture was extracted with Et₂O. The organic phase was dried with Na₂SO₄ filtered and evaporated to a yellow oil, 5.12 g yield 98%.

HPLC 98%, R_{T}=1.71 min (System. 10-97% MeCN 5 mM ammonium acetate over 3 min).

¹H NMR (400 MHz, CHLOROFORM-D) δ ppm 2.44 (t, J=7.0 Hz, 2H) 3.01 (t, J=5.4 Hz, 2H) 3.06 (t, J=7.0 Hz, 2H) 3.13 (s, 2H) 4.15 (t, J=5.4 Hz, 2H) 6.92-7.00 (m, 3H) 7.28-7.33 (m, 2H).

MS (ESI+) for C₁₂H₁₆NO₂ m/z 206 (M+H)⁺.

### Example 184

### 1-(4-Methoxyphenyl)-3-(6-methoxy-2,3,4,9-tetrahydro-1′H-spiro[beta-carboline-1,3′-pyrrolidin]-1′-yl)propan-1-one trifluoroacetate

6-Methoxy-2,3,4,9-tetrahydrospiro[beta-carboline-1,3′-pyrrolidine] hydrochloride (COMPARATIVE EXAMPLE 182, 0.050 g, 0.194 mmol) was suspended in MeCN. A few drops of DMSO were added until no unsolved material was present. 3-Chloro-1-(4-methoxyphenyl)propan-1-one (0.033 g, 0.253 mmol) and K₂CO₃ (0.035 g, 0.253 mmol) was added and the reaction mixture was agitated at room temperature for 16 h. Purification was performed using preparative LC (System A, 20-50% MeCN over 5 min) afforded 0.0174 g (21%) of a light brown gum.

HPLC 91% R_{T}=1.66 min (System A. 10-97% MeCN over 3 min), 89% R_{T}=1.47 min (System B. 10-97% MeCN over 3 min).

¹H NMR (270 MHz, METHANOL-D3) δ ppm 2.62-2.70 (m, 1H) 2.75-3.00 (m, 3H) 3.07 (t, J=6.06 Hz, 2H) 3.57-3.74 (m, 4H) 3.78 (s, 1H) 3.81 (s, 3H) 3.87 (s, 3H) 3.92-4.10 (m, 2H) 4.36 (d, J=13.98 Hz, 1H) 6.86 (dd, J=8.85, 2.41 Hz, 1H) 6.96-7.07 (m, 3H) 7.29 (d, J=8.78 Hz, 1H) 7.94-8.06 (m, 2H).

MS (ESI+) for C₂₅H₂₉N₃O₃ m/z 420 (M+H)⁺.

### Comparative Example 186

### Enantiomer (NB—The Chirality of the Compound is Relative)

### 6-Methoxy-2,3,4,9-tetrahydrospiro[beta-carboline-1,3′-pyrrolidine]

The title compound was provided from COMPARATIVE EXAMPLE 1 using chiral preparative HPLC.

Chiral HPLC 100%, R_{T}=9.9 min.

### Example 199

### 2-Acetyl-6-methoxy-1′-[2-(3-methoxyphenoxy)ethyl]-2,3,4,9-tetrahydrospiro[beta-carboline-1,3′-pyrrolidine]

The title compound was prepared from 6-methoxy-1′-[2-(3-methoxyphenoxy)ethyl]-2,3,4,9-tetrahydrospiro[beta-carboline-1,3′-pyrrolidine] (0.05 mmol) as described in General Synthetic Procedure D, Method B to afford 0.0054 g.

HPLC 100%, Rt=1.324 min.

¹H-NMR (500 MHz, DMSO-d₆) δ=2.25-2.29 (s, 3H), 2.41-4.29 (m, 12H), 4.33-4.39 (s, 2H), 6.56-6.62 (m, 3H), 6.77-6.82 (m, 1H), 6.97-7.01 (s, 1H), 7.18-7.24 (m, 1H), 7.27-7.31 (d, 1H, d=8.53 Hz)

MS (ESI+) m/z 450 (M+H)⁺.

### Example 200

### 2-Acetyl-8-methyl-1′-(2-phenoxyethyl)-2,3,4,9-tetrahydrospiro[beta-carboline-1,3′-pyrrolidine]

The title compound was prepared from EXAMPLE 124 according to General Synthetic Procedure D, Method B.

HPLC 100%

### Example 201

### 5,8-Dimethyl-1′-(2-phenoxyethyl)-2,3,4,9-tetrahydrospiro[beta-carboline-1,3′-pyrrolidine] trifluoroacetate

2-(3-Chloropropyl)-1,3-dioxolane (350 μL, 2.7 mmol) was added to (2,5-dimethylphenyl)hydrazine (362 mg, 2.7 mmol) in ethanol (25 mL) and water (5 mL) and the reaction was heated at 95° C. for 1 h and the solvent was than removed in vacuo. The crude was purified with preparative HPLC using acetonitrile-water gradients containing 0.1% triflouroacetic acid.

1-(2-Phenoxyethyl)pyrrolidin-3-one (COMPARATIVE EXAMPLE 183, 93 mg, 0.45 mmol) in acetic acid (1 mL) was added to 2-(4,7-dimethyl-1H-indol-3-yl)ethanamine (85.6 mg, 0.45 mmol) and the reaction was heated at 100° C. for 1 h, diluted with methanol (2 mL) and purified by preparative HPLC using acetonitrile-water gradients containing 0.1% triflouroacetic acid to afford 40.4 mg (24%).

HPLC 100%, R_{T}: 1.888 (10-97% MeCN over 3 min).

¹H NMR (270 MHz, Methanol-d₃) δ ppm 2.45 (s, 3H) 2.58 (s, 3H) 2.64-2.76 (m, 1H) 2.84-2.92 (m, 1H) 3.57-4.10 (m, 10H) 4.35-4.39 (m, 2H) 6.71 (d, J=7.18 Hz, 1H) 6.83-6.86 (m, 1H) 6.97 (t, J=7.92 Hz, 3H) 7.25-7.32 (m, 2H).

MS (ESI+) m/z 376 (M+H)⁺.

### Comparative Example 202

### tert-Butyl 6-methoxy-2,3,4,9-tetrahydro-1′H-spiro[beta-carboline-1,3′-pyrrolidine]-1′-carboxylate

A mixture of 5-methoxytryptamine hydrochloride (1.35 g 5.9 mmol) and tert-butyl 3-oxopyrrolidine-1-carboxylate 1.00 g, 5.4 mmol) was heated in HOAc at 75° C. for 5 h. The solvent was evaporated at reduced pressure and the residue was taken up between CHCl₃ and 50/50 1M HCl/brine, the organic phase was washed once with CHCl₃ and the combined organic phases where washed once with 80/20 brine/2M NaOH, dried (MgSO₄) and the solvent was removed at reduced pressure. The grey residue was chromatographed on a column of silica with initially two column volumes of CHCl₃ 100%, followed by CHCl₃/MeOH/aq conc NH₃ 95/5/0.2. The solvent from the pure fractions was evaporated at reduced pressure to give 1.41 g (73%) of the target compound as a white crispy foam.

HPLC 100%, R_{T}=1.67 min (System A. 10-97% MeCN), 100%, R_{T}=1.50 min (System B. 10-97% MeCN).

¹H NMR (400 MHz, MeOD) δ ppm 1.47 (d, J=11.17 Hz, 9H) 2.03 (dd, J=8.16, 4.89 Hz, 1H) 2.33-2.50 (m, 1H) 2.64-2.73 (m, 2H) 2.96-3.06 (m, 1H) 3.09-3.18 (m, 1H) 3.50-3.67 (m, 4H) 3.78 (s, 3H) 6.71 (dd, J=8.72, 2.45 Hz, 1H) 6.89 (d, J=2.26 Hz, 1H) 7.15 (d, J=8.78 Hz, 1H).

¹³C NMR (CD₃OD) δ 23.07, 28.77, 37.83, 38.53, 41.19, 45.54, 45.95, 56.25, 57.15, 57.62, 61.33, 62.12, 79.46, 80.99, 101.10, 110.59, 110.67, 112.41, 112.55, 128.60, 132.96, 135.79, 155.03, 156.41, 156.49.

MS (ESI+) m/z 358 (M+H)⁺.

### Example 206

### 1′-[2-(3-Isopropylphenoxy)ethyl]-6-methoxy-2,3,4,9-tetrahydrospiro[beta-carboline-1,3′-pyrrolidine]

The title compound was prepared from COMPARATIVE EXAMPLE 1 (66 mg, 0.26 mmol) and 2-(3-isopropylphenoxy)ethyl methanesulfonate (73 mg) as described in General Synthetic Procedure B, Method B to afford 0.0213 g.

HPLC 100%, Rt=1.762 min.

¹H-NMR (500 MHz, DMSO-d₆) δ=1.16-1.22 (d, 6H, J=6.70 Hz), 2.41-3.50 (m, 12H), 3.74-3.81 (s, 3H), 4.19-4.26 (s, 2H), 6.77-6.88 (m, 4H), 6.95-7.01 (s, 1H), 7.18-7.25 (m, 1H), 7.27-7.31 (d, 1H, J=8.53 Hz), 10.91-10.98 (s, 1H).

MS (ESI+) m/z 420 (M+H)⁺.

### Example 213

### 2-Acetyl-1′-[2-(4-ethylphenoxy)ethyl]-6-methoxy-2,3,4,9-tetrahydrospiro[beta-carboline-1,3′-pyrrolidine]

The title compound was prepared from 1′-[2-(4-ethylphenoxy)ethyl]-6-methoxy-2,3,4,9-tetrahydrospiro[beta-carboline-1,3′-pyrrolidine] (0.05 mmol) as described in General Synthetic Procedure D, Method B to afford 0.0057 g.

HPLC 100%, Rt=1.673 min.

¹H-NMR (500 MHz, DMSO-d₆) δ=2.4-4.29 (m, 14H), 3.73-3.81 (m, 6H), 4.30-4.35 (s, 2H), 6.79-6.82 (d, 1H, J=8.53 HZ), 6.90-6.95 (d, 2H, J=7.92 Hz), 6.97-7.01 (s, 1H), 7.11-7.17 (d, 2H, J=7.92 Hz), 7.27-7.32 (d, 1H, J=8.53 Hz).

MS (ESI+) m/z 448 (M+H)⁺.

### Example 214

### 2-Acetyl-6-methoxy-1′-(2-phenylethyl)-2,3,4,9-tetrahydrospiro[beta-carboline-1,3′-pyrrolidine]

The title compound was prepared from EXAMPLE 220 according to General Synthetic Procedure D, Method B.

HPLC 100%

### Example 215

### 2-Acetyl-6-methoxy-1′-[2-(2-methylphenoxy)ethyl]-2,3,4,9-tetrahydrospiro[beta-carboline-1,3′-pyrrolidine]

The title compound was prepared from 6-methoxy-1′-[2-(2-methylphenoxy)ethyl]-2,3,4,9-tetrahydrospiro[beta-carboline-1,3′-pyrrolidine] (0.05 mmol) as described in General Synthetic Procedure D, Method B to afford 0.0061 g.

HPLC 100%, Rt=1.483 min.

¹H-NMR (500 MHz, DMSO-d₆) δ=2.19-2.23 (s, 3H), 2.25-2.28 (s, 3H), 2.39-4.33 (m, 12H), 3.75-3.78 (s, 3H), 6.77-6.82 (d, 1H, J=8.53 Hz), 6.86-6.91 (m, 1H), 6.95-7.01 (m, 2H), 7.14-7.20 (m, 2H), 7.28-7.32 (d, 1H, J=9.13 Hz).

MS (ESI+) m/z 434 (M+H)⁺.

### Example 219

### 6-Methoxy-1′-(2-phenoxypropyl)-2,3,4,9-tetrahydrospiro[beta-carboline-1,3′-pyrrolidine]

The title compound was prepared from COMPARATIVE EXAMPLE 1 (66 mg, 0.26 mmol) and 2-phenoxypropyl methanesulfonate (65 mg) as described in General Synthetic Procedure B, Method B to afford 0.0267 g.

HPLC 100%, Rt=1.510 min.

¹H-NMR (500 MHz, DMSO-d₆) δ 1.47-1.53 (d, 3H), 2.48-3.15 (m, 1H), 3.92-3.98 (s, 3H), 4.85-4.94 (s, 1H), 6.96-7.00 (d, 1H, J=8.53 Hz), 7.09-7.19 (m, 3H), 7.43-7.52 (m, 3H).

MS (ESI+) m/z 392 (M+H)⁺.

### Example 220

### 6-Methoxy-1′-(2-phenylethyl)-2,3,4,9-tetrahydrospiro[beta-carboline-1,3′-pyrrolidine]

The title compound was prepared according to the same procedure as described for EXAMPLE 8.

HPLC 100%

### Example 223

### 6-Methoxy-1′-[2-(2-methoxyphenoxy)ethyl]-2,3,4,9-tetrahydrospiro[beta-carboline-1,3′-pyrrolidine]

The title compound was prepared from COMPARATIVE EXAMPLE 1 (66 mg, 0.26 mmol) and 2-(2-methoxyphenoxy)ethyl methanesulfonate (69 mg) as described in General Synthetic Procedure B, Method B to afford 0.0250 g.

HPLC 100%, Rt=1.416 min.

¹H-NMR (500 MHz, DMSO-d₆) δ 2.34-2.93 (m, 12H), 3.7-3.78 (d, 3H, J=18.53), 4.17-4.23 (s, 2H), 6.77-6.81 (m, 1H), 6.87-7.05 (m, 5H), 7.27-7.30 (m, 1H, J=8.79 Hz)

MS (ESI+) m/z 408 (M+H)⁺.

### Example 226

### 6-Methoxy-1′-[2-(2-naphthyloxy)ethyl]-2,3,4,9-tetrahydrospiro[beta-carboline-1,3′-pyrrolidine]

The title compound was prepared from COMPARATIVE EXAMPLE 1 (66 mg, 0.26 mmol) and 2-(2-naphthyloxy)ethyl methanesulfonate (75 mg) as described in General Synthetic Procedure B, Method B to afford 0.0203 g.

HPLC 100%, Rt=1.685 min.

¹H-NMR (500 MHz, DMSO-d₆) δ 2.40-3.50 (m, 12H), 3.73-3.79 (s, 3H), 4.32-4.42 (s, 2H), 6.77-6.82 (d, 1H, J=8.53 Hz), 6.96-7.01 (s, 1H), 7.18-7.24 (d, 1H, J=8.53 Hz), 7.27-7.31 (d, 1H, J=9.14 Hz), 7.43-7.40 (s, 2H), 7.45-7.50 (m, 1H), 7.79-7.88 (m, 3H).

MS (ESI+) m/z 428 (M+H)⁺.

### Example 228

### 6-Methoxy-1′-[2-(3-methoxyphenoxy)-1-methylethyl]-2,3,4,9-tetrahydrospiro[beta-carboline-1,3′-pyrrolidine]

The title compound was prepared from COMPARATIVE EXAMPLE 1 (66 mg, 0.26 mmol) and 2-(3-methoxyphenoxy)-1-methylethyl methanesulfonate (73 mg) as described in General Synthetic Procedure B, Method B to afford 0.0252 g.

HPLC 100%, Rt=1.531 min.

¹H-NMR (500 MHz, DMSO-d₆) δ 1.32-1.40 (s, 3H), 2.43-3.63 (m, 11H), 3.79-3.90 (d, 6H, J=15.07), 4.15-4.32 (m, 2H), 6.70-6.80 (m, 3H), 6.97-7.03 (m, 1H), 7.18-7.22 (s, 1H), 7.38-7.54 (m, 2H).

MS (ESI+) m/z 422 (M+H)⁺.

### Example 230

### 3-[6-Methoxy-1′-(2-phenoxyethyl)-4,9-dihydrospiro[beta-carboline-1,3′-pyrrolidin]-2(3H)-yl]propane-1,2-diol trifluoroacetate

K₂CO₃ (36.6 mg, 0.3 mmol) and 1-chloro-2,3-propanediole (17 μL, 0.2 mmol) were added to 6-methoxy-1′-(2-phenoxyethyl)-2,3,4,9-tetrahydrospiro[beta-carboline-1,3′-pyrrolidine] (EXAMPLE 51, 50.0 mg, 0.1 mmol) in acetonitrile (2 mL) and the reaction was heated at 75° C. for 16 h and than K₂CO₃ (36.6 mg, 0.3 mmol) and 1-chloro-2,3-propanediole (17 μL, 0.2 mmol) were added and after 24 h the mixture was let to rt and the product was purified by preparative HPLC using acetonitrile-water gradients containing 0.1% triflouroacetic acid to afford a yellow product 16.0 mg (35%).

HPLC 90%, R_{T}: 1.751 (10-97% MeCN over 3 min).

¹H NMR (270 MHz, Methanol-d₃) δ ppm 2.52-3.01 (m, 2H) 3.02-3.18 (m, 2H) 3.40-3.92 (m, 9H) 3.81 (s, 3H) 3.93-4.00 (m, 2H) 4.07 (d, J=3.96 Hz, 1H) 4.34 (t, J=4.82 Hz, 2H) 6.85 (dd, J=8.78, 2.35 Hz, 1H) 6.92-7.06 (m, 4H) 7.19-7.41 (m, 3H).

MS (ESI+) m/z 452 (M+H)⁺.

### Example 232

### 6-Methoxy-1′-(4-methoxybenzyl)-2,3,4,9-tetrahydrospiro[beta-carboline-1,3′-pyrrolidine] trifluoroacetate—enantiomer (NB—The chirality of the compound is relative)

6-Methoxy-2,3,4,9-tetrahydrospiro[beta-carboline-1,3′-pyrrolidine] hydrochloride (COMPARATIVE EXAMPLE 182, 0.054 g, 0.210 mmol) was suspended in MeCN. K₂CO₃ (0.038 g, 0.273 mmol) was added and the reaction mixture was cooled in an ice-bath. 1-(Chloromethyl)-4-methoxybenzene (0.036 g, 0.231 mmol) in MeCN (1 mL) was added and the reaction mixture was agitated at room temperature for 16 h. Purification was performed using preparative LC (System A, 15-45% MeCN over 5 min) afforded 0.0162 g (20%) of a yellow gum.

HPLC 98% R_{T}=1.62 min (System A. 10-97% MeCN over 3 min), 98% R_{T}=1.43 min (System B. 10-97% MeCN over 3 min).

¹H NMR (270 MHz, METHANOL-D3) δ ppm 2.59-2.89 (m, 2H) 3.04 (t, J=6.00 Hz, 2H) 3.52-3.74 (m, 5H) 3.80 (s, 6H) 3.90-3.99 (m, 1H) 4.36 (d, J=5.20 Hz, 2H) 6.86 (dd, J=8.78, 2.47 Hz, 1H) 6.93-7.02 (m, 3H) 7.29 (d, J=8.78 Hz, 1H) 7.40-7.51 (m, 2H).

MS (ESI+) for C₂₃H₂₇N₃O₂ m/z 378 (M+H)⁺.

### Example 233

### 1-(6-Methoxy-1′-(2-phenoxyethyl)-4,9-dihydrospiro[β-carboline-1,3′-pyrrolidin]-2(3H)-yl]-1-oxoacetone

Phosphouros oxychloride (49 μL, 0.5 mmol) was added to 6-methoxy-1′-(2-phenoxyethyl)-2,3,4,9-tetrahydrospiro[beta-carboline-1,3′-pyrrolidine] (EXAMPLE 51, 200 mg, 0.5 mmol) and puruvic acid (37 μL, 0.5 mmol) in THF (2 mL) at −15° C., followed by pyridine (128 μL, 1.6 mmol) and the mixture was allowed to stand 45 min at −15° C. and than let to rt. According to LC-MS 32% of the product was obtained.

HPLC 32%, R_{T}: 1.381 (10-97% MeCN over 3 min).

MS (ESI+) m/z 448 (M+H)⁺.

### Example 234

### 6-Methoxy-1′,2-bis(4-methoxybenzyl)-2,3,4,9-tetrahydrospiro[beta-carboline-1,3′-pyrrolidine]

6-Methoxy-2,3,4,9-tetrahydrospiro[beta-carboline-1,3′-pyrrolidine] hydrochloride (COMPARATIVE EXAMPLE 182, 0.050 g, 0.194 mmol) was suspended in MeCN. A few drops of DMSO were added until no unsolved material was present. 1-(Chloromethyl)-4-methoxybenzene (0.040 g, 0.253 mmol) and K₂CO₃ (0.035 g, 0.253 mmol) was added and the reaction mixture was agitated at room temperature for 16 h. Purification was performed using preparative LC (System A, 20-50% MeCN over 5 min) afforded 0.0123 g (13%) of a light brown gum.

HPLC 92% R_{T}=1.84 min (System A. 10-97% MeCN over 3 min), 93% R_{T}=1.63 min (System B. 10-97% MeCN over 3 min).

¹H NMR (270 MHz, METHANOL-D3) δ ppm 2.63-2.70 (m, 2H) 2.76-2.92 (m, 4H) 2.98 (s, 1H) 3.58 (d, J=8.66 Hz, 1H) 3.80 (d, J=4.82 Hz, 6H) 3.86 (s, 3H) 3.97 (t, J=13.42 Hz, 3H) 4.20 (d, J=14.35 Hz, 1H) 4.66 (s, 2H) 6.80 (dd, J=8.85, 2.41 Hz, 1H) 6.93 (d, J=2.35 Hz, 1H) 6.99-7.16 (m, 5H) 7.48 (d, J=8.78 Hz, 2H) 7.62 (d, J=8.78 Hz, 2H).

MS (ESI+) for C₃₁H₃₅N₃O₃ m/z 498 (M+H)⁺.

Preparation of a Pharmaceutical Composition

### Example 235

### Preparation of Tablets

| Ingredients | mg/tablet |
|---|---|
| 1. Active compound of formula (I) | 10.0 |
| 2. Cellulose, microcrystalline | 57.0 |
| 3. Calcium hydrogen phosphate | 15.0 |
| 4. Sodium starch glycolate | 5.0 |
| 5. Silicon dioxide, colloidal | 0.25 |
| 6. Magnesium stearate | 0.75 |

The active ingredient 1 is mixed with ingredients 2, 3, 4 and 5 for about 10 minutes. The magnesium stearate is then added, and the resultant mixture is mixed for about 5 minutes and compressed into tablet form with or without film-coating.

Biological Methods

Experimental Methods

Primary Screening and IC₅₀ Determination

Chinese hamster ovary cells (CHO), cell line (ES-410-F) purchased from Euroscreen, stably expressing the human GHSR seeded in 96 well plates are pre-loaded with Fluo-4AM fluorescent dye for 60 min before addition of test compounds (5 μM for primary screen). Fluorescent intensity is recorded using a Fluorometric imaging plate reader (FLIPR 98R 96-well format, Molecular Devices) and inhibition of the peak response evoked by ghrelin (EC₇₀ concentration) is calculated.

Potency (IC₅₀) determinations are performed utilizing the same functional assay as described for primary screening, applying the compounds in the concentration range of 170 pM to 10 μM or 340 pM to 20 μM.

Biology Summary

The calculation of the functional Kᵢ values for the inhibitors was performed by use of Activity Base. The Kᵢ value is calculated from IC₅₀ using the Cheng Prushoff equation (with reversible inhibition that follows the Michaelis-Menten equation): Kᵢ=IC₅₀ (1+[S]/Kₘ) [Cheng, Y. C.; Prushoff, W. H. Biochem. Pharmacol. 1973, 22, 3099-3108]. The compounds of formula (I) exhibit Kᵢ values for the GHSR in the range from 10 nM to ≧5 μM. See for example table:

| Example No. | GHSR-human Ki (nM) |
|---|---|
| 10 | 60 nM |
| 11 | 572 nM |
| 30 | 59 nM |

A number of embodiments of the invention have been described. Nevertheless, it will be understood that various modifications may be made without departing from the spirit and scope of the invention.

1. A compound of Formula (I) 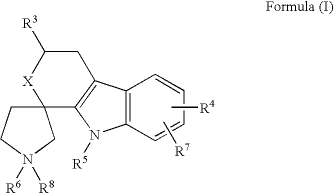
wherein
X is O or NR, wherein R is selected from hydrogen, C₁₋₆-alkyl, hydroxy-C₁₋₆-alkyl, C₂₋₆-alkenyl, C₂₋₆-acyl, hydroxy-C₂₋₆-acyl, C₁₋₆-alkylcarbamoyl, di-C₁₋₆-alkylcarbamoyl, C₂₋₆-alkenylcarbamoyl, C₃₋₈-cycloalkylcarbamoyl, C₁₋₆-alkylsulfonyl, N-glycylcarbonyl, C₁₋₆-alkyl ester of N-glycylcarbonyl, C₁₋₆-alkyl ester of N-glycylacetyl, carbamoyl-C₁₋₆-alkyl, N—C₁₋₆-alkylcarbamoyl-C₁₋₆-alkyl, N,N—C₁₋₆-dialkylcarbamoyl-C₁₋₆-alkyl, N,N—C₁₋₆-dialkylcarbamoylamino-C₁₋₆-alkyl, C₁₋₆-alkoxy-C₂₋₆-acylamino-C₁₋₆-alkyl, 3-amino-1,2-dioxocyclobut-3-ene-4-ylamino-C₁₋₆-alkyl, 3-C₁₋₆-alkoxy-1,2-dioxocyclobut-3-ene-4-ylamino-C₁₋₆-alkyl, cyano-C₁₋₆-alkyl, C₁₋₆-alkoxyhydroxyalkyl, carboxy-C₁₋₆-alkyl, C₁₋₆-alkoxycarbonyl-C₁₋₆-alkyl, C₁₋₆-alkoxy-C₁₋₆-alkyl, C₁₋₆-alkoxy-C₁₋₆-alkoxy-C₁₋₆-alkyl, aryl-C₁₋₆-alkylamino-C₂₋₆-acyl, C₁₋₆-alkoxycarbonyl-C₁₋₆-alkylamino-C₂₋₆-acyl, carboxy-C₁₋₆-alkylamino-C₂₋₆-acyl, C₂₋₆-acyl-C₂₋₆-acyl, aryloxy-C₁₋₆-alkyl, C₁₋₆-alkylsulfonylamino-C₁₋₆-alkyl, C₁₋₆-alkoxycarbonyl-C₂₋₆-acyl, C₁₋₆-alkoxy-C₂₋₆-acyl, C₁₋₆-alkylthio-C₂₋₆-acyl, di-C₁₋₆-alkylamino-C₂₋₆-acyl, heteroarylcarbamoyl, C₁₋₆-alkoxycarbonyl, heteroaryl-C₂₋₆-acyl, C₁₋₆-alkylsulfonyl-C₂₋₆-acyl, heterocyclyl-C₂₋₆-acyl, C₁₋₆-alkoxy-C₁₋₆-alkylamino-C₂₋₆-acyl, carboxy-C₂₋₆-acyl, amino-C₂₋₆-acyl, C₁₋₆-alkylamino-C₂₋₆-acyl, carbamoyl-C₁₋₆-alkylamino-C₂₋₆-acyl, heterocyclyl-C₁₋₆-alkyl, heteroaryl-C₁₋₆-alkyl, carbamoylamino-C₁₋₆-alkyl, hydroxy-C₂₋₆-acylcarbamoyl, C₁₋₆-alkylcarbamoyl-C₁₋₆-alkylamino-C₁₋₆-alkyl, C₁₋₆-alkoxycarbonyl-C₁₋₆-alkylamino-C₁₋₆-alkyl, amino-C₂₋₆-acylamino-C₂₋₆-acyl, C₁₋₆-alkoxy-C₂₋₆-acylamino-C₂₋₆-acyl, amino-C₂₋₆-acylamino-C₁₋₆-alkyl, amino-C₂₋₆-acylamino-C₁₋₆-alkyl, heterocyclylcarbonylamino-C₁₋₆-alkyl, C₂₋₆-acylamino-C₁₋₆-alkyl, amino-C₂₋₆-acylamino-C₂₋₆-acyl, C₂₋₆-acylamino-C₂₋₆-acyl, hydroxy-C₁₋₆-alkylamino-C₂₋₆-acyl, C₁₋₆-alkoxycarbonyl-C₁₋₆-alkyl, amino-C₁₋₆-alkyl, carboxy-C₁₋₆-alkyl, 2-(3-hydroxy-1,2-dioxocyclobut-3-ene-4-yl)amino-C₁₋₆-alkyl, heteroarylcarbonylamino-C₁₋₆-alkyl, carboxyamino-C₁₋₆-alkyl, N,N-di-C₁₋₆-alkylamino-C₂₋₆-acylamino-C₁₋₆-alkyl, dihydroxy-C₁₋₆-alkyl, C₂₋₆-acylcarbonyl, C₁₋₆-alkoxybenzyl, and CO—CH₂—R⁶, wherein the aryl group is optionally substituted by one or more of C₁₋₆-alkoxy, the heteroaryl group is optionally substituted by one or more of C₁₋₆-alkyl and the heterocyclyl is optionally substituted by one or more of oxo;
Y is O, S, NH, CH₂, CO, or a single bond;
R¹ is hydrogen or C₁₋₃-alkyl;
R² is C₃₋₈-cycloalkyl, hexahydro-N-phthalimidyl, an aryl or heteroaryl ring optionally substituted by one or more of C₁₋₆-alkyl, halogen, methylenedioxy, C₁₋₆-alkoxy, halo-C₁₋₆-alkoxy, C₁₋₆-alkylsulfonyl, or cyano;
R³ is hydrogen;
R⁴ is hydrogen, C₁₋₆-alkyl, C₁₋₆-alkoxy, or halogen;
R⁵ is hydrogen or C₁₋₆-alkyl;
R⁶ is either bonded to X via a methylene and a carbonyl group, or R⁶ is hydroxy-C₁₋₆-alkyl or C₁₋₆-alkoxycarbonyl-C₁₋₆-alkyl;
R⁷ is hydrogen or C₁₋₆-alkyl;
R⁸ is —CH(R¹)—(CHOH)ₘ—[(CH(R⁹)]ₙ—Y—R², —CH(R¹)—(CH═CH)ₒ—Y—R², hydrogen or C₁₋₆-alkyl;
R⁹ is hydrogen or C₁₋₆-alkyl;
m is 0 or 1;
n is 0, 1, or 2;
o is 0 or 1;
with the proviso that when Y is a single bond, then R² is 5-methyl-3-indolyl, 3-indolyl, cyclohexyl, 2,3-dihydro-1,4-benzodioxin-2-yl, phenyl, 2-methoxyphenyl, 4-difluoromethoxyphenyl, or 3,4-methylenedioxyphenyl;
and pharmaceutically acceptable salts, hydrates, solvates, geometrical isomers, tautomers, optical isomers, and prodrug forms thereof.

2. The compound according to claim 1, wherein R is selected from acetyl, allyl, allylcarbamoyl, aminoacetyl, 2-(3-amino-1,2-dioxocyclobut-3-ene-4-ylamino)ethyl, 3-amino-3-methyl-n-butyryl, benzylaminoacetyl, n-butylcarbamoyl, carbamoylmethyl, carbamoylmethylaminoacetyl, 3-carbamoyl-n-propyl, carbethoxy, carbethoxyacetyl, 4-carbethoxy-n-butyl, carbethoxymethyl, 3-carbethoxy-n-propyl, carbomethoxyacetyl, 4-carbomethoxy-n-butyryl, 4-carboxy-n-butyl, 3-carboxy-n-propionyl, 3-carboxy-n-propyl, 3-cyano-n-propyl, cyclohexylcarbamoyl, N,N-diethylcarbamoylmethyl, diisopropylaminoacetyl, 3,4-dimethoxybenzylaminoacetyl, dimethylaminoacetyl, 2-(N,N-dimethylcarbamoylamino)ethyl, 3,5-dimethylisoxazol-4-ylcarbamoyl, 1,4-dioxo-n-pentyl, 2-(3-ethoxy-1,2-dioxocyclobut-3-ene-4-ylamino)ethyl, ethylcarbamoyl, 4-ethylcarbamoyl-n-butyl, 3-ethylcarbamoyl-n-propyl, ethyl ester of N-glycylacetyl, ethyl ester of N-glycylcarbonyl, N-ethyl-N-methylcarbamoyl, ethylthioacetyl, N-glycylacetyl, N-glycylcarbonyl, hydrogen, hydroxyacetyl, 2-hydroxyisobutyl, 2-hydroxyethyl, 2-hydroxy-3-methoxy-n-propyl, 2-hydroxy-n-propyl, 1-imidazolylacetyl, methoxyacetyl, 2-(methoxyacetylamino)ethyl, 2-(2-methoxyethoxy)ethyl, 2-methoxyethylaminoacetyl, 3-methoxy-n-propyl, methyl, methylaminoacetyl, methylsulfonyl, methylsulfonylacetyl, 2-methylsulfonylaminoethyl, 4-morpholinylacetyl, 2-(4-morpholinyl)ethyl, 3-oxo-1-piperazinylacetyl, 2-phenoxyethyl, 1-piperazinylacetyl, 2-pyridylmethyl, 2-thienylcarbamoyl, 2-carbamoylaminoethyl, hydroxyacetylcarbamoyl, 2-(N-methylcarbamoylmethylamino)ethyl, 2-carbomethoxymetylaminoethyl, 2-amino-2-methylpropionamidoacetyl, methoxyacetylaminoacetyl, 2-(2-amino-2-methylpropionamido)ethyl, 2-aminoacetylaminoethyl, 2-(4-morpholinylcarbonylamino)ethyl, 2-acetylaminoethyl, aminoacetylaminoacetyl, acetylaminoacetyl, 2-hydroxyethylaminoacetyl, carbomethoxymethyl, 2-aminoethyl, carboxymethyl, 2-(3-hydroxy-1,2-dioxocyclobut-3-ene-4-yl)aminoethyl, 2-(2-furylcarbonylamino)ethyl, 2-(5-isoxazolylcarbonylamino)ethyl, 2-carboxyaminoethyl, 2-(2-morpholinylcarbonylamino)ethyl, 2-N,N-dimethylaminoacetylaminoethyl, 4-phenoxy-n-butyl, 2,3-dihydroxy-n-propyl, acetylcarbonyl, and 4-methoxybenzyl.

3. The compound of claim 1, wherein R¹ is hydrogen or methyl.

4. The compound of claim 1, wherein R² is selected from N-hexahydrophthalimidyl, cyclohexyl, 2,3-dihydro-1,4-benzodioxin-2-yl; a phenyl or indole ring optionally substituted by one or more of methyl, ethyl, fluoro, chloro, methylenedioxy, difluoromethoxy, methylsulfonyl, methoxy, cyano, isopropyl; and naphthyl.

5. The compound of claim 1, wherein R² is selected from N-hexahydrophthalimidyl, cyclohexyl, 2,3-dihydro-1,4-benzodioxin-2-yl, phenyl, 2-methylphenyl, 2-fluorophenyl, 4-fluorophenyl, 3-chlorophenyl, 4-chlorophenyl, 3,4-methylenedioxyphenyl, 2-methoxyphenyl, 4-methoxyphenyl, 2-cyanophenyl, 4-cyanophenyl, 4-ethylphenyl, 4-difluoromethoxyphenyl, 4-methylsulfonylphenyl, 4-carbamoylphenyl, indolyl, 5-methyl-3-indolyl, 3-methoxyphenyl, 3-isopropylphenyl, and naphthyl.

6. The compound of claim 1, wherein R⁴ is selected from hydrogen, bromo, fluoro, methyl, and methoxy.

7. The compound of claim 1, wherein R⁵ is hydrogen or methyl.

8. The compound of claim 1, wherein R⁶ is hydroxymethyl or carbomethoxymethyl.

9. The compound of claim 1, wherein R⁷ is hydrogen or methyl.

10. The compound of claim 1, wherein R⁸ is hydrogen or methyl.

11. The compound of claim 1, wherein R⁹ is hydrogen or methyl.

12. The compound of claim 1, wherein the compound is selected from:
6-Methoxy-1′-[2-(5-methyl-1H-indol-3-yl)ethyl]-2,3,4,9-tetrahydrospiro[beta-carboline-1,3′-pyrrolidine];
N-Cyclohexyl-6-methoxy-1′-(2-phenoxyethyl)-4,9-dihydrospiro[beta-carboline-1,3′-pyrrolidine]-2(3H)-carboxamide;
2-Acetyl-1′-[2-(4-fluorophenoxy)ethyl]-6-methoxy-2,3,4,9-tetrahydro spiro [beta-carboline-1,3′-pyrrolidine];
2-Acetyl-6-methoxy-1′-(2-phenoxyethyl)-2,3,4,9-tetrahydrospiro[beta-carboline-1,3′-pyrrolidine];
1′-(2-Cyclohexylethyl)-6-methoxy-2,3,4,9-tetrahydrospiro[beta-carboline-1,3′-pyrrolidine];
1′-(2-Phenoxyethyl)-2,3,4,9-tetrahydrospiro[beta-carboline-1,3′-pyrrolidine];
6-Methoxy-1′-(2-phenoxyethyl)-2,3,4,9-tetrahydrospiro[beta-carboline-1,3′-pyrrolidine];
7-Fluoro-1′-(2-phenoxyethyl)-2,3,4,9-tetrahydrospiro[beta-carboline-1,3′-pyrrolidine];
6-Methoxy-2-(methylsulfonyl)-1′-(2-phenoxyethyl)-2,3,4,9-tetrahydrospiro[beta-carboline-1,3′-pyrrolidine];
1′-[2-(1,3-Benzodioxol-5-yl)ethyl]-6-methoxy-2,3,4,9-tetrahydrospiro[beta-carboline-1,3′-pyrrolidine];
6-Methoxy-1′-[2-(3-methoxyphenoxy)ethyl]-2,3,4,9-tetrahydrospiro[beta-carboline-1,3′-pyrrolidine];
1′-[2-(2-Fluorophenoxy)ethyl]-6-methoxy-2,3,4,9-tetrahydrospiro[beta-carboline-1,3′-pyrrolidine];
6-Methoxy-1′-[2-(2-methoxyphenoxy)ethyl]-2,3,4,9-tetrahydrospiro[beta-carboline-1,3′-pyrrolidine];
1′-[2-(4-Chlorophenoxy)ethyl]-6-methoxy-2,3,4,9-tetrahydrospiro[beta-carboline-1,3′-pyrrolidine];
6-Methoxy-1′-(1-methyl-2-phenoxyethyl)-2,3,4,9-tetrahydrospiro[beta-carboline-1,3′-pyrrolidine];
4-[2-(6-Methoxy-2,3,4,9-tetrahydro-1′H-spiro[beta-carboline-1,3′-pyrrolidin]-1′-yl)ethoxy]benzonitrile;
2-Acetyl-6-methoxy-1′-[2-(2-methoxyphenoxy)ethyl]-2,3,4,9-tetrahydrospiro[beta-carboline-1,3′-pyrrolidine];
2-Acetyl-1′-[2-(2-fluorophenoxy)ethyl]-6-methoxy-2,3,4,9-tetrahydrospiro[beta-carboline-1,3′-pyrrolidine];
1′-[2-(4-Fluorophenoxy)ethyl]-4,9-dihydro-3H-spiro [pyrano[3,4-b]indole-1,3′-pyrrolidine];
1′-(2-Phenoxyethyl)-4,9-dihydro-3H-spiro[pyrano[3,4-b]indole-1,3′-pyrrolidine];
N-Ethyl-6-methoxy-1′-(2-phenoxyethyl)-4,9-dihydrospiro[beta-carboline-1,3′-pyrrolidine]-2(3H)-carboxamide;
Ethyl ({[6-methoxy-1′-(2-phenoxyethyl)-4,9-dihydrospiro[beta-carboline-1,3′-pyrrolidin]-2(3H)-yl]carbonyl}amino)acetate;
N-Allyl-6-methoxy-1′-(2-phenoxyethyl)-4,9-dihydrospiro[beta-carboline-1,3′-pyrrolidine]-2(3H)-carboxamide;
N-Butyl-6-methoxy-1′-(2-phenoxyethyl)-4,9-dihydro spiro [beta-carboline-1,3′-pyrrolidine]-2(3H)-carboxamide;
N′-{2-[6-Methoxy-1′-(2-phenoxyethyl)-4,9-dihydrospiro[beta-carboline-1,3′-pyrrolidin]-2(3H)-yl]ethyl}-N,N-dimethylurea trifluoroacetate;
2-Methoxy-N-{2-[6-methoxy-1′-(2-phenoxyethyl)-4,9-dihydrospiro[beta-carboline-1,3′-pyrrolidin]-2(3H)-yl]ethyl}acetamide trifluoroacetate;
3-Amino-4-({2-[6-methoxy-1′-(2-phenoxyethyl)-4,9-dihydrospiro[beta-carboline-1,3′-pyrrolidin]-2(3H)-yl]ethyl}amino)cyclobut-3-ene-1,2-dione trifluoroacetate;
Ethyl ({[7-fluoro-1′-(2-phenoxyethyl)-4,9-dihydrospiro[beta-carboline-1,3′-pyrrolidin]-2(3H)-yl]carbonyl}amino)acetate trifluoroacetate;
Ethyl ({[6-methyl-1′-(2-phenoxyethyl)-4,9-dihydrospiro[beta-carboline-1,3′-pyrrolidin]-2(3H)-yl]carbonyl}amino)acetate trifluoroacetate;
4-[6-Methoxy-1′-(2-phenoxyethyl)-4,9-dihydrospiro[beta-carboline-1,3′-pyrrolidin]-2(3H)-yl]butanenitrile;
2-[6-Methoxy-1′-(2-phenoxyethyl)-4,9-dihydrospiro[beta-carboline-1,3′-pyrrolidin]-2(3H)-yl]acetamide;
N-Ethyl-4-[6-methoxy-1′-(2-phenoxyethyl)-4,9-dihydrospiro[beta-carboline-1,3′-pyrrolidin]-2(3H)-yl]butanamide;
1-Methoxy-3-[6-methoxy-1′-(2-phenoxyethyl)-4,9-dihydrospiro[beta-carboline-1,3′-pyrrolidin]-2(3H)-yl]propan-2-ol trifluoroacetate;
N-Ethyl-6-methoxy-1′-(2-phenoxyethyl)-4,9-dihydrospiro[beta-carboline-1,3′-pyrrolidine]-2(3H)-carboxamide;
Ethyl 4-[6-methoxy-1′-(2-phenoxyethyl)-4,9-dihydrospiro[beta-carboline-1,3′-pyrrolidin]-2(3H)-yl]butanoate;
6-Methoxy-2-(3-methoxypropyl)-1′-(2-phenoxyethyl)-2,3,4,9-tetrahydrospiro[beta-carboline-1,3′-pyrrolidine];
3-Ethoxy-4-({2-[6-methoxy-1′-(2-phenoxyethyl)-4,9-dihydrospiro[beta-carboline-1,3′-pyrrolidin]-2(3H)-yl]ethyl}amino)cyclobutane-1,2-dione trifluoroacetate;
6-Methoxy-2-methyl-1′-(2-phenoxyethyl)-2,3,4,9-tetrahydrospiro[beta-carboline-1,3′-pyrrolidine];
6-Methoxy-2-[2-(2-methoxyethoxy)ethyl]-1′-(2-phenoxyethyl)-2,3,4,9-tetrahydrospiro[beta-carboline-1,3′-pyrrolidine];
2-[6-Methoxy-1′-(2-phenoxyethyl)-4,9-dihydrospiro[beta-carboline-1,3′-pyrrolidin]-2(3H)-yl]-2-oxoethanol trifluoroacetate;
Ethyl 5-[6-methoxy-1′-(2-phenoxyethyl)-4,9-dihydrospiro[beta-carboline-1,3′-pyrrolidin]-2(3H)-yl]pentanoate;
4-[6-Methoxy-1′-(2-phenoxyethyl)-4,9-dihydrospiro[beta-carboline-1,3′-pyrrolidin]-2(3H)-yl]butanoic acid;
2-Allyl-6-methoxy-1′-(2-phenoxyethyl)-2,3,4,9-tetrahydrospiro[beta-carboline-1,3′-pyrrolidine];
6-Methoxy-1′-(2-phenoxyethyl)-2,3,4,9-tetrahydrospiro[beta-carboline-1,3′-pyrrolidine];
N-Ethyl-5-[6-methoxy-1′-(2-phenoxyethyl)-4,9-dihydrospiro[beta-carboline-1,3′-pyrrolidin]-2(3H)-yl]pentanamide;
N-Benzyl-2-[6-methoxy-1′-(2-phenoxyethyl)-4,9-dihydrospiro[beta-carboline-1,3′-pyrrolidin]-2(3H)-yl]-2-oxoethanamine trifluoroacetate;
Methyl ({2-[6-methoxy-1′-(2-phenoxyethyl)-4,9-dihydrospiro[beta-carboline-1,3′-pyrrolidin]-2(3H)-yl]-2-oxoethyl}amino)acetate trifluoroacetate;
Ethyl [6-methoxy-1′-(2-phenoxyethyl)-4,9-dihydrospiro[beta-carboline-1,3′-pyrrolidin]-2(3H)-yl]acetate;
N-(3,4-Dimethoxybenzyl)-2-[6-methoxy-1′-(2-phenoxyethyl)-4,9-dihydrospiro[beta-carboline-1,3′-pyrrolidin]-2(3H)-yl]-2-oxoethanamine trifluoroacetate;
5-[6-Methoxy-1′-(2-phenoxyethyl)-4,9-dihydrospiro[beta-carboline-1,3′-pyrrolidin]-2(3H)-yl]-5-oxopentan-2-one trifluoro acetate;
6-Methoxy-1′,2-bis(2-phenoxyethyl)-2,3,4,9-tetrahydrospiro[beta-carboline-1,3′-pyrrolidine];
N-{2-[6-Methoxy-1′-(2-phenoxyethyl)-4,9-dihydrospiro[beta-carboline-1,3′-pyrrolidin]-2(3H)-yl]ethyl}methanesulfonamide trifluoroacetate;
1-[6-Methoxy-1′-(2-phenoxyethyl)-4,9-dihydrospiro[beta-carboline-1,3′-pyrrolidin]-2(3H)-yl]propan-2-ol trifluoroacetate;
Ethyl 3-[6-methoxy-1′-(2-phenoxyethyl)-4,9-dihydrospiro[beta-carboline-1,3′-pyrrolidin]-2(3H)-yl]-3-oxopropanoate trifluoroacetate;
2-[6-Methoxy-1′-(2-phenoxyethyl)-4,9-dihydrospiro[beta-carboline-1,3′-pyrrolidin]-2(3H)-yl]ethanol;
1-[6-Methoxy-1′-(2-phenoxyethyl)-4,9-dihydrospiro[beta-carboline-1,3′-pyrrolidin]-2(3H)-yl]-2-methylpropan-2-ol trifluoroacetate;
Methyl 5-[6-methoxy-1′-(2-phenoxyethyl)-4,9-dihydrospiro[beta-carboline-1,3′-pyrrolidin]-2(3H)-yl]-5-oxopentanoate trifluoroacetate;
6-Methoxy-2-(methoxyacetyl)-1′-(2-phenoxyethyl)-2,3,4,9-tetrahydrospiro[beta-carboline-1,3′-pyrrolidine] trifluoroacetate;
6-Fluoro-1′-(2-phenoxyethyl)-2,3,4,9-tetrahydrospiro[beta-carboline-1,3′-pyrrolidine] trifluoroacetate;
1′-[2-(4-Fluorophenoxy)ethyl]-6-methoxy-2,3,4,9-tetrahydrospiro[beta-carboline-1,3′-pyrrolidine];
4-{2-[6-Methoxy-2,3,4,9-tetrahydro-1′H-spiro[beta-carboline-1,3′-pyrrolidin]-1′-yl]ethoxy}benzonitrile trifluoroacetate;
2-[(Ethylthio)acetyl]-6-methoxy-1′-(2-phenoxyethyl)-2,3,4,9-tetrahydrospiro[beta-carboline-1,3′-pyrrolidine] trifluoroacetate;
N-Isopropyl-N-{2-[6-methoxy-1′-(2-phenoxyethyl)-4,9-dihydrospiro[beta-carboline-1,3′-pyrrolidin]-2(3H)-yl]-2-oxoethyl}propan-2-amine trifluoroacetate;
4-[6-Methoxy-1′-(2-phenoxyethyl)-4,9-dihydrospiro[beta-carboline-1,3′-pyrrolidin]-2(3H)-yl]butanamide;
1′-[2-(4-Cyanophenoxy)ethyl]-N-(3,5-dimethylisoxazol-4-yl)-6-methoxy-4,9-dihydrospiro[beta-carboline-1,3′-pyrrolidine]-2(3H)-carboxamide trifluoroacetate;
N,N-Diethyl-2-[6-methoxy-1′-(2-phenoxyethyl)-4,9-dihydrospiro[beta-carboline-1,3′-pyrrolidin]-2(3H)-yl]acetamide;
Ethyl 6-methoxy-1′-(2-phenoxyethyl)-4,9-dihydrospiro[beta-carboline-1,3′-pyrrolidine]-2(3H)-carboxylate trifluoroacetate;
2-(1H-Imidazol-1-ylacetyl)-6-methoxy-1′-(2-phenoxyethyl)-2,3,4,9-tetrahydrospiro[beta-carboline-1,3′-pyrrolidine] trifluoroacetate;
N-[2-(6-Methoxy-2,3,4,9-tetrahydro-1′H-spiro[beta-carboline-1,3′-pyrrolidin]-1′-yl)ethyl]aniline trifluoroacetate;
6,8-Dimethyl-1′-(2-phenoxyethyl)-2,3,4,9-tetrahydrospiro[beta-carboline-1,3′-pyrrolidine] trifluoroacetate;
6-Methoxy-1′-(2-phenoxyethyl)-2,3,4,9-tetrahydrospiro[beta-carboline-1,3′-pyrrolidine];
6-Methyl-1′-(2-phenoxyethyl)-2,3,4,9-tetrahydrospiro[beta-carboline-1,3′-pyrrolidine];
6-Methoxy-1′-[2-(4-methoxyphenoxy)ethyl]-2,3,4,9-tetrahydrospiro[beta-carboline-1,3′-pyrrolidine] trifluoroacetate;
1′-[2-(4-Fluorophenoxy)ethyl]-6-methoxy-2,3,4,9-tetrahydrospiro[beta-carboline-1,3′-pyrrolidine] trifluoroacetate;
5-[6-Methoxy-1′-(2-phenoxyethyl)-4,9-dihydrospiro[beta-carboline-1,3′-pyrrolidin]-2(3H)-yl]pentanoic acid;
N-Ethyl-6-methoxy-1′-(2-phenoxyethyl)-4,9-dihydrospiro[beta-carboline-1,3′-pyrrolidine]-2(3H)-carboxamide;
6-Methoxy-2-[(methylsulfonyl)acetyl]-1′-(2-phenoxyethyl)-2,3,4,9-tetrahydrospiro[beta-carboline-1,3′-pyrrolidine] trifluoroacetate;
6-Bromo-1′-(2-phenoxyethyl)-2,3,4,9-tetrahydrospiro[beta-carboline-1,3′-pyrrolidine];
6-Methoxy-1′-[2-(4-methoxyphenoxy)ethyl]-N-2-thienyl-4,9-dihydrospiro[beta-carboline-1,3′-pyrrolidine]-2(3H)-carboxamide trifluoroacetate;
1′-[2-(4-Chlorophenoxy)ethyl]-6-methoxy-2,3,4,9-tetrahydrospiro[beta-carboline-1,3′-pyrrolidine] trifluoroacetate;
{2-[6-Methoxy-1′-(2-phenoxyethyl)-4,9-dihydrospiro[beta-carboline-1,3′-pyrrolidin]-2(3H)-yl]-2-oxoethyl}dimethylamine trifluoroacetate;
1′-[2-(3-Chlorophenoxy)ethyl]-6-methoxy-2,3,4,9-tetrahydrospiro[beta-carboline-1,3′-pyrrolidine];
6-Methoxy-1′-[2-(2-methylphenoxy)ethyl]-2,3,4,9-tetrahydrospiro[beta-carboline-1,3′-pyrrolidine];
6-Methoxy-1′-[3-(2-methoxyphenyl)propyl]-2,3,4,9-tetrahydrospiro[beta-carboline-1,3′-pyrrolidine];
1′-[2-(4-Ethylphenoxy)ethyl]-6-methoxy-2,3,4,9-tetrahydrospiro[beta-carboline-1,3′-pyrrolidine];
6-Methoxy-1′-(2-phenoxyethyl)-2-(piperazin-1-ylacetyl)-2,3,4,9-tetrahydrospiro[beta-carboline-1,3′-pyrrolidine] trifluoroacetate;
2-[3-(6-Methoxy-2,3,4,9-tetrahydro-1′H-spiro[beta-carboline-1,3′-pyrrolidin]-1′-yl)propyl]hexahydro-1H-isoindole-1,3(2H)-dione;
Ethyl ({[8-methyl-1′-(2-phenoxyethyl)-4,9-dihydrospiro[beta-carboline-1,3′-pyrrolidin]-2(3H)-yl]carbonyl}amino)acetate trifluoroacetate;
N-(2-Methoxyethyl)-2-[6-methoxy-1′-(2-phenoxyethyl)-4,9-dihydrospiro[beta-carboline-1,3′-pyrrolidin]-2(3H)-yl]-2-oxoethanamine trifluoroacetate;
({[6-Methoxy-1′-(2-phenoxyethyl)-4,9-dihydrospiro[beta-carboline-1,3′-pyrrolidin]-2(3H)-yl]carbonyl}amino)acetic acid;
Methyl 3-[6-methoxy-1′-(2-phenoxyethyl)-4,9-dihydrospiro[beta-carboline-1,3′-pyrrolidin]-2(3H)-yl]-3-oxopropanoate trifluoroacetate;
4-[6-Methoxy-1′-(2-phenoxyethyl)-4,9-dihydrospiro[beta-carboline-1,3′-pyrrolidin]-2(3H)-yl]-4-oxobutanoic acid trifluoroacetate;
N-(3,5-Dimethylisoxazol-4-yl)-6-methoxy-1′-[2-(4-methoxyphenoxy)ethyl]-4,9-dihydrospiro[beta-carboline-1,3′-pyrrolidine]-2(3H)-carboxamide trifluoroacetate;
{2-[6-Methoxy-1′-(2-phenoxyethyl)-4,9-dihydrospiro[beta-carboline-1,3′-pyrrolidin]-2(3H)-yl]-2-oxoethyl}amine trifluoroacetate;
{2-[6-Methoxy-1′-(2-phenoxyethyl)-4,9-dihydrospiro[beta-carboline-1,3′-pyrrolidin]-2(3H)-yl]-2-oxoethyl}methylamine trifluoroacetate;
7-Methoxy-14-oxo-16-(2-phenoxyethyl)-3,13-diaza-16-azoniapentacyclo[14.2.1.0˜1,13˜.0˜2,10˜.0˜4,9˜]nonadeca-2(10),4,6,8-tetraene chloride;
N2-{2-[6-Methoxy-1′-(2-phenoxyethyl)-4,9-dihydrospiro[beta-carboline-1,3′-pyrrolidin]-2(3H)-yl]-2-oxoethyl}glycinamide trifluoroacetate-N,N-diethylethanamine (1:1);
6-Methoxy-1′-[2-(phenylthio)ethyl]-2,3,4,9-tetrahydrospiro[beta-carboline-1,3′-pyrrolidine] trifluoroacetate;
6-Methoxy-2-(morpholin-4-ylacetyl)-1′-(2-phenoxyethyl)-2,3,4,9-tetrahydrospiro[beta-carboline-1,3′-pyrrolidine] trifluoroacetate;
1′-[2-(Benzyloxy)ethyl]-6-methoxy-2,3,4,9-tetrahydrospiro[beta-carboline-1,3′-pyrrolidine] trifluoroacetate;
{3-[6-Methoxy-1′-(2-phenoxyethyl)-4,9-dihydrospiro[beta-carboline-1,3′-pyrrolidin]-2(3H)-yl]-1,1-dimethyl-3-oxopropyl}amine hydrochloride;
7-Methoxy-1′-(2-phenoxyethyl)-2,3,4,9-tetrahydrospiro[beta-carboline-1,3′-pyrrolidine] trifluoroacetate;
1′-[4-(Difluoromethoxy)benzyl]-6-methoxy-2,3,4,9-tetrahydrospiro[beta-carboline-1,3′-pyrrolidine];
1′-[2-(1H-Indol-3-yl)ethyl]-6-methoxy-2,3,4,9-tetrahydrospiro[beta-carboline-1,3′-pyrrolidine] trifluoroacetate;
6-Methoxy-2-(2-morpholin-4-ylethyl)-1′-(2-phenoxyethyl)-2,3,4,9-tetrahydrospiro[beta-carboline-1,3′-pyrrolidine];
6-Methoxy-1′-(2-phenoxyethyl)-2-(pyridin-2-ylmethyl)-2,3,4,9-tetrahydrospiro[beta-carboline-1,3′-pyrrolidine];
6-Methoxy-1′-(3-phenoxypropyl)-2,3,4,9-tetrahydrospiro[beta-carboline-1,3′-pyrrolidine];
6-Methoxy-1′,2-bis(2-phenoxyethyl)-2,3,4,9-tetrahydrospiro[beta-carboline-1,3′-pyrrolidine];
6-Methoxy-1′-{2-[4-(methylsulfonyl)phenoxy]ethyl}-2,3,4,9-tetrahydrospiro[beta-carboline-1,3′-pyrrolidine];
8-Methyl-1′-(2-phenoxyethyl)-2,3,4,9-tetrahydrospiro[beta-carboline-1,3′-pyrrolidine];
4-{2-[6-Methoxy-1′-(2-phenoxyethyl)-4,9-dihydrospiro[beta-carboline-1,3′-pyrrolidin]-2(3H)-yl]-2-oxoethyl}piperazin-2-one trifluoroacetate;
1′-Benzyl-6-methoxy-2,3,4,9-tetrahydrospiro[beta-carboline-1,3′-pyrrolidine] hydrochloride;
({2-[6-Methoxy-1′-(2-phenoxyethyl)-4,9-dihydrospiro[beta-carboline-1,3′-pyrrolidin]-2(3H)-yl]-2-oxoethyl}amino)acetic acid trifluoroacetate;
3-(6-Methoxy-2,3,4,9-tetrahydro-1′H-spiro[beta-carboline-1,3′-pyrrolidin]-1′-yl)-1-phenylpropan-1-one trifluoroacetate;
2-[2-(6-Methoxy-2,3,4,9-tetrahydro-1′H-spiro[beta-carboline-1,3′-pyrrolidin]-1′-yl)ethoxy]benzonitrile;
N-Ethyl-6-methoxy-N,9-dimethyl-1′-(2-phenoxyethyl)-4,9-dihydrospiro[beta-carboline-1,3′-pyrrolidine]-2(3H)-carboxamide trifluoroacetate;
6-Methoxy-9-methyl-1′-(2-phenoxyethyl)-2,3,4,9-tetrahydrospiro[beta-carboline-1,3′-pyrrolidine] trifluoroacetate;
1-(6-Methoxy-2,3,4,9-tetrahydro-1′H-spiro[beta-carboline-1,3′-pyrrolidin]-1′-yl)-3-phenylpropan-2-ol trifluoroacetate;
6-Methoxy-2-(4-phenoxybutyl)-2,3,4,9-tetrahydrospiro[beta-carboline-1,3′-pyrrolidine];
1′-(2,3-Dihydro-1,4-benzodioxin-2-ylmethyl)-6-methoxy-2,3,4,9-tetrahydrospiro[beta-carboline-1,3′-pyrrolidine];
6-Methoxy-1′-methyl-2-(2-phenoxyethyl)-2,3,4,9-tetrahydrospiro[beta-carboline-1,3′-pyrrolidine] trifluoroacetate;
N-{2-[6-Methoxy-1′-(2-phenoxyethyl)-4,9-dihydrospiro[beta-carboline-1,3′-pyrrolidin]-2(3H)-yl]ethyl}urea;
N-Glycoloyl-6-methoxy-1′-(2-phenoxyethyl)-4,9-dihydrospiro[beta-carboline-1,3′-pyrrolidine]-2(3H)-carboxamide;
N′-{2-[6-Methoxy-1′-(2-phenoxyethyl)-4,9-dihydrospiro[beta-carboline-1,3′-pyrrolidin]-2(3H)-yl]ethyl}-N,N-dimethylurea;
2-({2-[6-Methoxy-1′-(2-phenoxyethyl)-4,9-dihydrospiro[beta-carboline-1,3′-pyrrolidin]-2(3H)-yl]ethyl}amino)-N-methylacetamide;
Methyl ({2-[6-methoxy-1′-(2-phenoxyethyl)-4,9-dihydrospiro[beta-carboline-1,3′-pyrrolidin]-2(3H)-yl]ethyl}amino)acetate;
2-Amino-N-{2-[6-methoxy-1′-(2-phenoxyethyl)-4,9-dihydrospiro[beta-carboline-1,3′-pyrrolidin]-2(3H)-yl]-2-oxoethyl}-2-methylpropanamide;
2-Methoxy-N-{2-[6-methoxy-1′-(2-phenoxyethyl)-4,9-dihydrospiro[beta-carboline-1,3′-pyrrolidin]-2(3H)-yl]-2-oxoethyl}acetamide;
2-Amino-N-{2-[6-methoxy-1′-(2-phenoxyethyl)-4,9-dihydrospiro[beta-carboline-1,3′-pyrrolidin]-2(3H)-yl]ethyl}-2-methylpropanamide;
2-Amino-N-{2-[6-methoxy-1′-(2-phenoxyethyl)-4,9-dihydrospiro[beta-carboline-1,3′-pyrrolidin]-2(3H)-yl]ethyl}acetamide;
2-Methoxy-N-{2-[6-methoxy-1′-(2-phenoxyethyl)-4,9-dihydrospiro[beta-carboline-1,3′-pyrrolidin]-2(3H)-yl]ethyl}acetamide;
N-{2-[6-Methoxy-1′-(2-phenoxyethyl)-4,9-dihydrospiro[beta-carboline-1,3′-pyrrolidin]-2(3H)-yl]ethyl}morpholine-4-carboxamide trifluoroacetate;
N-{2-[6-Methoxy-1′-(2-phenoxyethyl)-4,9-dihydrospiro[beta-carboline-1,3′-pyrrolidin]-2(3H)-yl]ethyl}acetamide trifluoroacetate;
2-[6-Methoxy-1′-(2-phenoxyethyl)-4,9-dihydrospiro[beta-carboline-1,3′-pyrrolidin]-2(3H)-yl]acetamide;
2-Amino-N-{2-[6-methoxy-1′-(2-phenoxyethyl)-4,9-dihydrospiro[beta-carboline-1,3′-pyrrolidin]-2(3H)-yl]-2-oxoethyl}acetamide trifluoroacetate;
N-{2-[6-Methoxy-1′-(2-phenoxyethyl)-4,9-dihydrospiro[beta-carboline-1,3′-pyrrolidin]-2(3H)-yl]-2-oxoethyl}acetamide trifluoroacetate;
2-({2-[6-Methoxy-1′-(2-phenoxyethyl)-4,9-dihydrospiro[beta-carboline-1,3′-pyrrolidin]-2(3H)-yl]-2-oxoethyl}amino)ethanol trifluoroacetate;
Methyl ({2-[6-methoxy-1′-(2-phenoxyethyl)-4,9-dihydrospiro[beta-carboline-1,3′-pyrrolidin]-2(3H)-yl]-2-oxoethyl}amino)acetate trifluoroacetate;
{2-[6-Methoxy-1′-(2-phenoxyethyl)-4,9-dihydrospiro[beta-carboline-1,3′-pyrrolidin]-2(3H)-yl]-2-oxoethyl}amine trifluoroacetate;
2-[6-Methoxy-1′-(2-phenoxyethyl)-4,9-dihydrospiro[beta-carboline-1,3′-pyrrolidin]-2(3H)-yl]ethanol;
1,2-Bis(2-hydroxyethyl)-6-methoxy-1′-(2-phenoxyethyl)-2,3,4,9-tetrahydrospiro[beta-carboline-1,3′-pyrrolidine] trifluoroacetate;
6-Methoxy-1′,2-bis(2-methoxy-2-oxoethyl)-1′-(2-phenoxyethyl)-2,3,4,9-tetrahydrospiro[beta-carboline-1,3′-pyrrolidine] bromide;
Methyl [6-methoxy-1′-(2-phenoxyethyl)-4,9-dihydrospiro[beta-carboline-1,3′-pyrrolidin]-2(3H)-yl]acetate;
1′-[(1S)-2-(4-Fluorophenoxy)-1-methylethyl]-6-methoxy-2,3,4,9-tetrahydrospiro[beta-carboline-1,3′-pyrrolidine] trifluoroacetate;
6-Methoxy-1′-(1-methyl-2-phenoxyethyl)-2,3,4,9-tetrahydrospiro[beta-carboline-1,3′-pyrrolidine] trifluoroacetate;
2-[6-Methoxy-1′-(2-phenoxyethyl)-4,9-dihydrospiro[beta-carboline-1,3′-pyrrolidin]-2(3H)-yl]ethanamine;
[6-Methoxy-1′-(2-phenoxyethyl)-4,9-dihydrospiro[beta-carboline-1,3′-pyrrolidin]-2(3H)-yl]acetic acid acetate;
3-Hydroxy-4-({2-[(1S)-6-methoxy-1′-(2-phenoxyethyl)-4,9-dihydrospiro[beta-carboline-1,3′-pyrrolidin]-2(3H)-yl]ethyl}amino)cyclobut-3-ene-1,2-dione trifluoroacetate;
1-[6-Methoxy-2,3,4,9-tetrahydro-1′H-spiro[beta-carboline-1,3′-pyrrolidin]-1′-yl]-3-phenoxypropan-2-ol trifluoroacetate;
6-Methoxy-1′-[(2E)-3-phenylprop-2-en-1-yl]-2,3,4,9-tetrahydrospiro[beta-carboline-1,3′-pyrrolidine] trifluoroacetate;
6-Methoxy-1′-(3-phenylpropyl)-2,3,4,9-tetrahydrospiro[beta-carboline-1,3′-pyrrolidine] trifluoroacetate;
N-{2-[6-Methoxy-1′-(2-phenoxyethyl)-4,9-dihydrospiro[beta-carboline-1,3′-pyrrolidin]-2(3H)-yl]ethyl}-2-furamide;
N-{2-[6-Methoxy-1′-(2-phenoxyethyl)-4,9-dihydrospiro[beta-carboline-1,3′-pyrrolidin]-2(3H)-yl]ethyl}isoxazole-5-carboxamide trifluoroacetate;
2-Hydroxy-N-{2-[6-methoxy-1′-(2-phenoxyethyl)-4,9-dihydrospiro[beta-carboline-1,3′-pyrrolidin]-2(3H)-yl]ethyl}acetamide trifluoroacetate;
N-{2-[6-methoxy-1′-(2-phenoxyethyl)-4,9-dihydrospiro[beta-carboline-1,3′-pyrrolidin]-2(3H)-yl]ethyl}morpholine-2-carboxamide trifluoroacetate;
2-(Dimethylamino)-N-{2-[6-methoxy-1′-(2-phenoxyethyl)-4,9-dihydrospiro[beta-carboline-1,3′-pyrrolidin]-2(3H)-yl]ethyl}acetamide trifluoroacetate;
2-[6-Methoxy-1′-(2-phenoxyethyl)-4,9-dihydrospiro[beta-carboline-1,3′-pyrrolidin]-2(3H)-yl]ethanamine;
1-(4-Methoxyphenyl)-3-(6-methoxy-2,3,4,9-tetrahydro-1′H-spiro[beta-carboline-1,3′-pyrrolidin]-1′-yl)propan-1-one trifluoroacetate;
2-Acetyl-6-methoxy-1′-[2-(3-methoxyphenoxy)ethyl]-2,3,4,9-tetrahydrospiro[beta-carboline-1,3′-pyrrolidine];
2-Acetyl-8-methyl-1′-(2-phenoxyethyl)-2,3,4,9-tetrahydrospiro[beta-carboline-1,3′-pyrrolidine];
5,8-Dimethyl-1′-(2-phenoxyethyl)-2,3,4,9-tetrahydrospiro[beta-carboline-1,3′-pyrrolidine] trifluoroacetate;
1′-[2-(3-Isopropylphenoxy)ethyl]-6-methoxy-2,3,4,9-tetrahydrospiro[beta-carboline-1,3′-pyrrolidine];
2-Acetyl-1′-[2-(4-ethylphenoxy)ethyl]-6-methoxy-2,3,4,9-tetrahydrospiro[beta-carboline-1,3′-pyrrolidine];
2-Acetyl-6-methoxy-1′-(2-phenylethyl)-2,3,4,9-tetrahydrospiro[beta-carboline-1,3′-pyrrolidine];
2-Acetyl-6-methoxy-1′-[2-(2-methylphenoxy)ethyl]-2,3,4,9-tetrahydrospiro[beta-carboline-1,3′-pyrrolidine];
6-Methoxy-1′-(2-phenoxypropyl)-2,3,4,9-tetrahydro spiro [beta-carbo line-1,3′-pyrrolidine];
6-Methoxy-1′-(2-phenylethyl)-2,3,4,9-tetrahydrospiro[beta-carboline-1,3′-pyrrolidine];
6-Methoxy-1′-[2-(2-methoxyphenoxy)ethyl]-2,3,4,9-tetrahydrospiro[beta-carboline-1,3′-pyrrolidine];
6-Methoxy-1′-[2-(2-naphthyloxy)ethyl]-2,3,4,9-tetrahydrospiro[beta-carboline-1,3′-pyrrolidine];
6-Methoxy-1′-[2-(3-methoxyphenoxy)-1-methylethyl]-2,3,4,9-tetrahydrospiro[beta-carboline-1,3′-pyrrolidine];
3-[6-Methoxy-1′-(2-phenoxyethyl)-4,9-dihydrospiro[beta-carboline-1,3′-pyrrolidin]-2(3H)-yl]propane-1,2-diol trifluoroacetate;
6-Methoxy-1′-(4-methoxybenzyl)-2,3,4,9-tetrahydrospiro[beta-carboline-1,3′-pyrrolidine] trifluoroacetate;
1-[6-Methoxy-1′-(2-phenoxyethyl)-4,9-dihydrospiro[p-carboline-1,3′-pyrrolidin]-2(3H)-yl]-1-oxoacetone; and
6-Methoxy-1′,2-bis(4-methoxybenzyl)-2,3,4,9-tetrahydro spiro [beta-carbo line-1,3′-pyrrolidine].

13. A process for the preparation of a compound according to claim 1, which process comprises at least one of the following reaction sequences a₁, a₂, a₃, b-p): 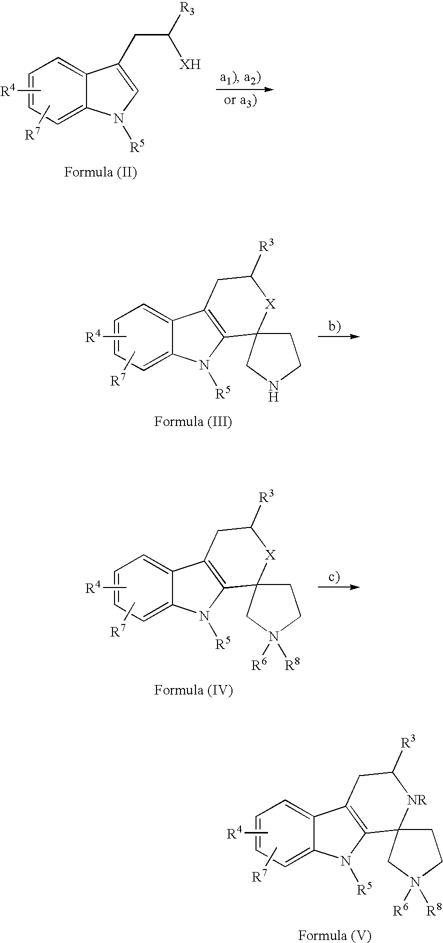
a1) the reaction of a compound of Formula (II) with N-Boc-3-pyrrolidinone and subsequent acidic hydrolysis;
a2) the reaction of a compound of Formula (II) with N-benzyl-3-pyrrolidinone and the subsequent hydrogenolysis;
a3) the reaction of a compound of Formula (II) with 1-(phenoxyethyl)pyrrolidin-3-one;
b) the reaction of a compound of Formula (III) with R²—Y—[(CH(R⁹)]ₙ—(CHOH)ₘ—CH(R¹)-LG or R²—Y—(CH═CH)ₙ—CH(R¹)-LG;
c) reaction of a compound of Formula (IV) with acetic anhydride, an isocyanate or an alkylating agent;
wherein R, Y, R¹, R¹, R³, R⁴, R⁵, R⁶, R⁷, R⁸, R⁹, m, n, and o are as defined in claim 1 and LG is a leaving group; 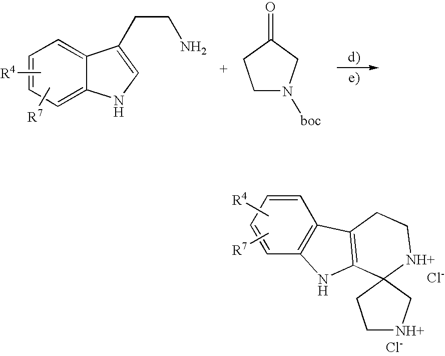
d) treatment with acetic acid;
e) treatment with hydrochloric acid in dioxane; 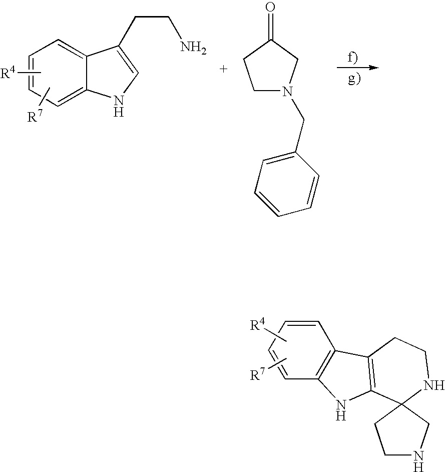
f) treatment with acetic acid;
g) treatment with hydrogen in the presence of palladium hydroxide; 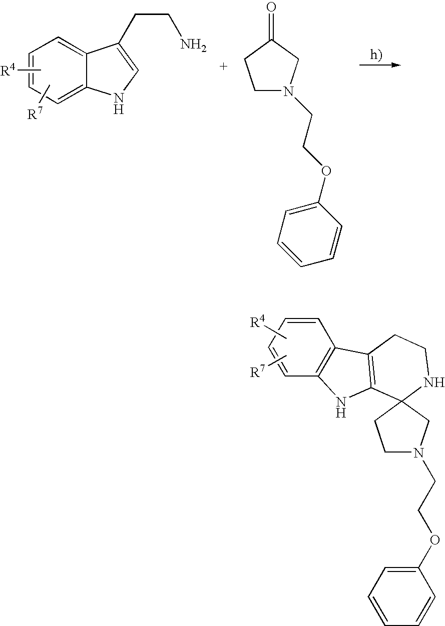
h) treatment with acetic acid; 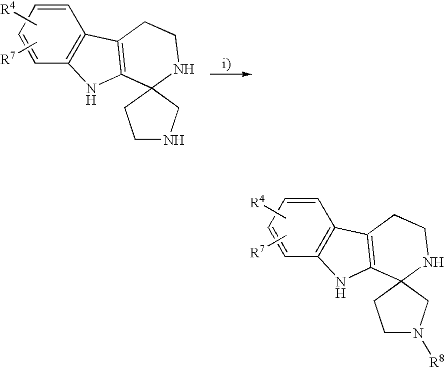
i) treatment with R⁸—Br, R⁸—Cl or R⁸—OMs; 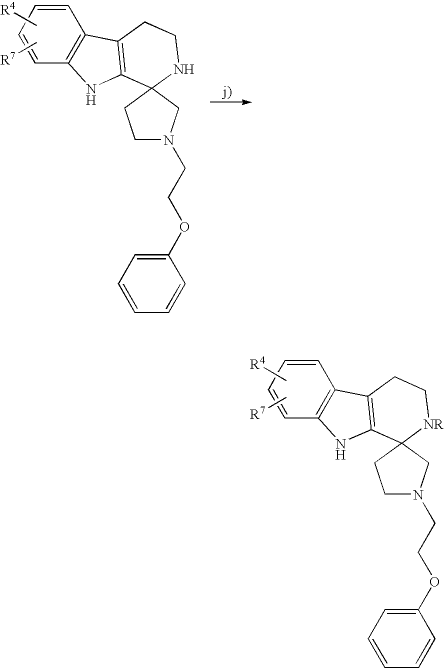
j) treatment with an isocyanate; 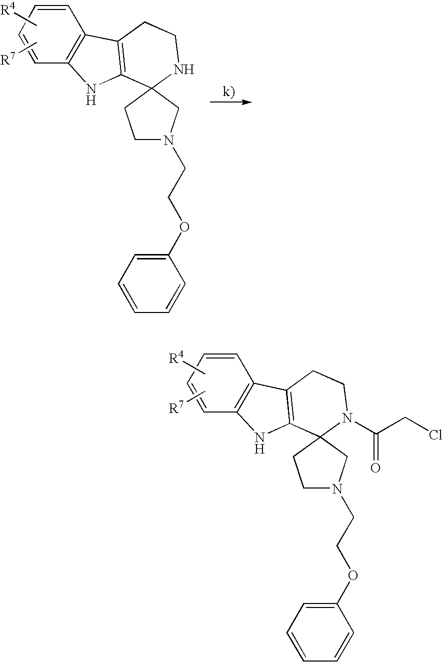
k) acylation with chloroacetyl chloride; 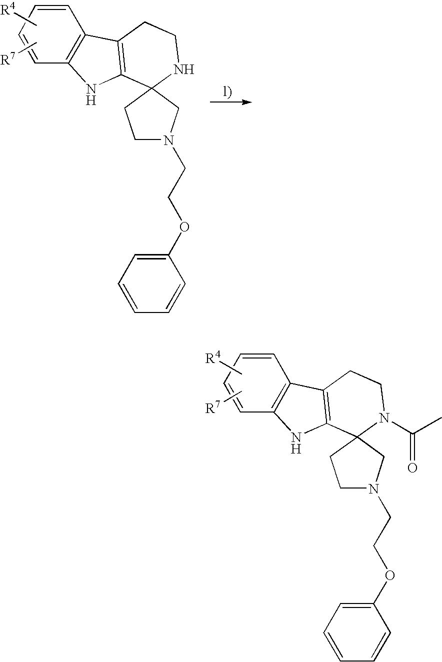
l) acylation with acetic anhydride; 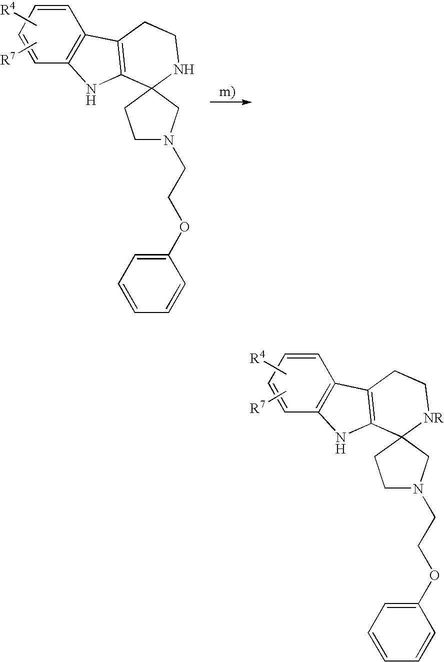
m) alkylation with R-LG; 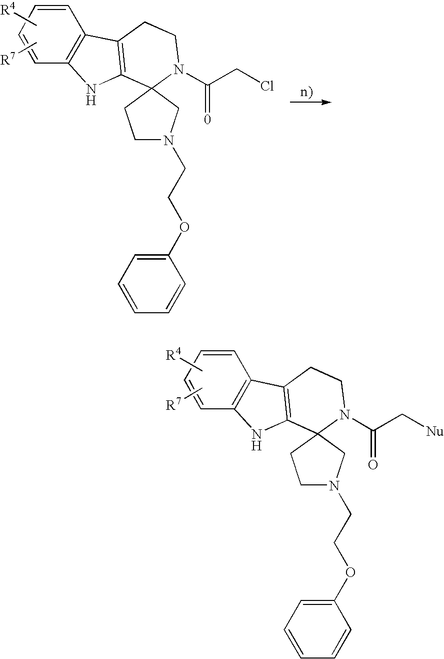
n) reaction with a nucleophile Nu; 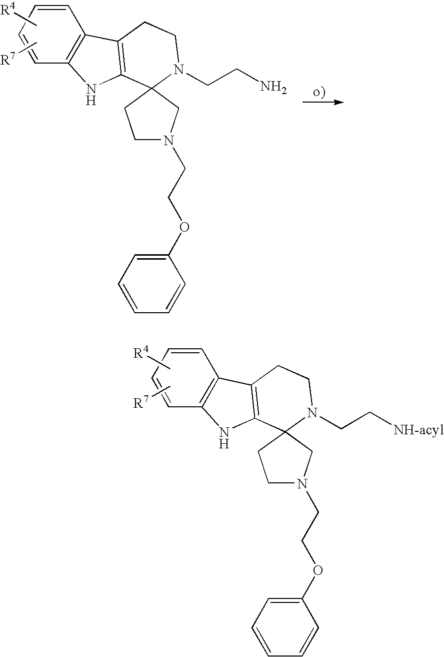
o) reaction with a carboxylic acid in the presence of a coupling agent; 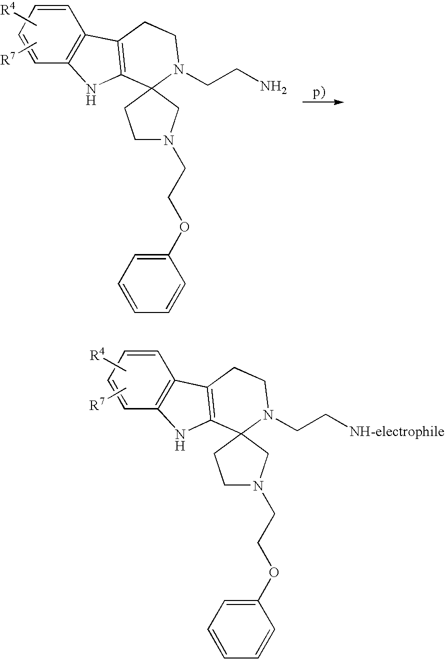
p) reaction with an electrophile.

14. A pharmaceutical formulation comprising a compound according to claim 1 as an active ingredient, in combination with a pharmaceutically acceptable diluent or carrier.

15. The pharmaceutical formulation according to claim 14, wherein the pharmaceutical formulation comprises an amount of the compound of claim 1 that is effective for the prophylaxis or treatment of a GHSR receptor-related disorder.

16. A method for the prophylaxis or treatment of a GHSR receptor-related disorder, which comprises administering to a subject in need of such treatment an effective amount of a compound according to claim 1.

17. The method according to claim 16, wherein the disorder is selected from obesity and related disorders; cardiovascular diseases; acromegaly; and cancer.

18. The method of claim 17, wherein the obesity-related disorder is diabetes type II, dyslipidemia, or Prader-Willi syndrome.

19. The method of claim 17, wherein the cardiovascular disease is atherosclerotic vascular disease, angina pectoris, myocardial infarction, or stroke.

20. The method of claim 17, wherein the disorder is acromegaly.

21. The method of claim 17, wherein the cancer is breast cancer, lung cancer, prostate cancer, thyroid cancer, or endocrine pituary carcinomas.

22. A method for modulating GHSR receptor activity, which comprises administering to a subject in need of such treatment an effective amount of a compound according to claim 1.

23. The method of claim 22, wherein modulating GHSR receptor activity comprises inhibiting GHSR receptor activity.

24. The method of claim 22, wherein modulating GHSR receptor activity comprises promoting GHSR receptor activity.

25. A method for suppressing food intake, which comprises administering to a subject in need of such treatment an effective amount of a compound according to claim 1.

26. A method for suppressing appetite, which comprises administering to a subject in need of such treatment an effective amount of a compound according to claim 1.

27. A method for reducing weight, which comprises administering to a subject in need of such treatment an effective amount of a compound according to claim 1.

28. A method for reducing weight gain, which comprises administering to a subject in need of such treatment an effective amount of a compound according to claim 1.

29. A method for increasing food intake, which comprises administering to a subject in need of such treatment an effective amount of a compound according to claim 1.

30. A method for increasing appetite, which comprises administering to a subject in need of such treatment an effective amount of a compound according to claim 1.

31. A method for increasing weight, which comprises administering to a subject in need of such treatment an effective amount of a compound according to claim 1.

32. A method for increasing weight gain, which comprises administering to a subject in need of such treatment an effective amount of a compound according to claim 1.

33. A method for preparing a pharmaceutical composition, the method comprising combining a compound according to claim 1 with a pharmaceutically acceptable carrier.
